(19) United States
(12) Patent Application Publication
(10) Pub. No.: US 2023/0046416 A1
(43) Pub. Date: Feb. 16, 2023

(54) Chemically Induced Association and Dissociation of Therapeutic FC Compositions and Chemically Induced Dimerization of T Cell Engager with Human Serum Albumin

(71) Applicant: Soteria Biotherapeutics, Inc., San Francisco, CA (US)

(72) Inventors: Zachary B. Hill, San Mateo, CA (US); Alexander J. Martinko, San Francisco, CA (US); James A. Wells, Burlingame, CA (US); Meghan Zubradt, Belmont, CA (US); Erin F. Simonds, San Francisco, CA (US)

(73) Assignee: Soteria Biotherapeutics, Inc., San Francisco, CA (US)

(21) Appl. No.: 17/788,418

(22) PCT Filed: Dec. 23, 2020
(86) PCT No.: PCT/US2020/066865

§ 371 Date: Jun. 23, 2022

Related U.S. Application Data
(60) Provisional application No. 62/953,003, filed on Dec. 23, 2019.
(60) Provisional application No. 62/952,984, filed on Dec. 23, 2019.

(51) Int. Cl. C07K 16/28

(52) U.S. Cl. CPC C07K 16/2803; C07K 16/2809; C07K 2317/60; C07K 2317/92; C07K 2319/30; C07K 2319/31

(57) ABSTRACT

The present disclosure provides a system that enables precise temporal control of the serum half-life a therapeutic moiety by inducing the association or disassociation of the therapeutic moiety with an Fc domain by a small molecule. The present disclosure also provides a system that enables precise control of the serum half- life a T cell engager domain by incorporating a chemically induced dimerizer (CID). One half of the CID is fused to a T cell engager, and the other half of the CID is fused to a HSA binding domain. Addition or removal of a small molecule induces association or dissociation of the T cell engager with HSA, thereby enabling precise temporal control of the serum half-life the T cell engager.

### CROSS REFERENCE TO RELATED APPLICATIONS

This application claims priority to U.S. Provisional Applications 62/953,003, filed Dec. 23, 2019 and 62/952,984, filed Dec. 23, 2019, the contents of which are both expressly incorporated by reference in their entirety.

### BACKGROUND OF THE INVENTION

T cell engagers are antibody derived therapeutics that transiently tether T cells via the T cell receptor complex (TCR) to surface antigens on tumor cells. This leads to activation of T cells and direction of T cell induced lysis of the attached target tumor cells. The therapeutic potential of a T cell engager was demonstrated by blinatumomab, a CD19/CD13-bispecific T cell engager approved for the treatment of adult patients with relapsed/refractory acute lymphoblastic leukemia. Despite success of the T cell-engaging therapy, one of the shorting comings of existing T cell engagers is short serum half-life.

Improvements have been made to address the short serum half-life of a T cell engager, for example, by fusing the T cell engager to human serum albumin (HSA) or an Fc domain (Merlot et al., Future Med Chem. 2015; 7:553-556; Kontermann et al., Chem Biotechnol. Pharm Biotechnol. 2011;22:868-876).

Human serum albumin (HSA) (molecular mass ~67 kDa) is the most abundant protein in plasma, present at about 50 mg/ml, and has a half-life of around 20 days in humans. HSA serves to maintain plasma pH, contributes to colloidal blood pressure, functions as carrier of many metabolites and fatty acids, and serves as a major drug transport protein in plasma. Noncovalent association with albumin extends the elimination half-time of short lived proteins. For example, a recombinant fusion of an albumin binding domain to a Fab fragment resulted in an in vivo clearance of 25- and 58-fold and a half-life extension of 26- and 37-fold when administered intravenously to mice and rabbits respectively as compared to the administration of the Fab fragment alone (Dennis et al., J Biol Chem. 2002;277(38):35035-43). In another example, when insulin is acylated with fatty acids to promote association with albumin, a protracted effect is observed when injected subcutaneously in rabbits or pigs (Kurtzhals et al., Biochem. J. 1995; 312: 725-731). Together, these studies demonstrate a linkage between albumin binding and prolonged action.

Fc-based fusion proteins are composed of an immunoglobin Fc domain that is directly linked to another peptide. The fused partner can be any other proteinaceous molecule of interest, such as a ligand that activates upon interaction with a cell-surface receptor, a peptidic antigen against a challenging pathogen or a ‘bait’ protein to identify binding partners assembled in a protein microarray. Most frequently though, the fused partners have significant therapeutic potential, and they are attached to an Fc-domain to endow the hybrids with a number of additional beneficial biological and pharmacological properties. One of the most important beneficial properties is that the presence of the Fc domain markedly increases their plasma half-life, which prolongs therapeutic activity, owing to its interaction with the salvage neonatal Fc-receptor (FcRn; Roopenian & Akilesh, Nat Rev Immunol. 2007;7(9):715-25), as well as to the slower renal clearance for larger sized molecules (Kontermann, Curr Opin Biotechnol. 2011; 22(6):868-76). The attached Fc domain also enables these molecules to interact with Fc-receptors (FcRs) found on immune cells, a feature that is particularly important for their use in oncological therapies and vaccines (Nimmerjahn & Ravetch, Nat Rev Immunol. 2008;8(1):34-47). From a biophysical perspective, the Fc domain folds independently and can improve the solubility and stability of the partner molecule both in vitro and in vivo, while from a technological viewpoint, the Fc region allows for easy cost-effective purification by protein-G/A affinity chromatography during manufacture (Carter, Exp Cell Res. 2011;317:1261-1269).

Despite efforts and progress made in extending the serum half-life of biologics by fusing them to Fc-domains, thus far the extension of serum half-life is not tunable. The present invention meets the need of developing more advanced therapies by providing a system that enables precise temporal control of the serum half-life of biologics, and in doing so enabling safer and more efficacious dosing of the biologics to patients.

### BRIEF SUMMARY OF THE INVENTION

The present invention provides a tunable control system for serum half-life of a T cell engager. In one aspect, the present invention provides a composition comprising (1) a heterodimeric Fc fusion protein which comprises a first monomer comprising a first chemically induced dimerizer (CID) domain and a first Fc domain of an IgG wherein said first CID domain is covalently linked to said first Fc domain, and a second monomer comprising a second Fc domain of said IgG; and (2) a fusion protein moiety comprising a second CID domain and a therapeutic moiety, wherein said second CID domain is covalently linked to said therapeutic moiety at N or C terminus. In the presence of a CID small molecule, the first CID domain and the CID second domain form a complex of first CID domain-CID small molecule-second CID domain.

In some embodiments, the CID small molecule is selected from the group consisting of FK1012, rimiducid, FK506, FKCsA, Rapamycin, Rapamycin analogs, Courmermycin, Gibberellin, HaXS, TMP-tag, ABT-737.

In some embodiments, the first CID domain-CID small molecule-second CID domain is selected from the group of complexes consisting of FKBP-FK1012-FKBP, variant FKBP-rimiducid-variant FKBP, FKBP-FK506-Calcineurin, FKBP-FKCsA-CyP-Fas, FKBP-Rapamycin-FRB, variant FKBP-Rapamycin analogs-variant FRB, GyrB-Courmermycin-GyrB, GAI-Gibberellin-GID1, SNAP-tag-HaXS-HaloTag, eDHFR-TMP-tag-HaloTag and AZ1-ABT-737-BCL-xL, wherein the first CID domain and the second CID domain can swap positions within the complex.

In some embodiments, the first CID domain comprises a heavy chain variable domain and a light chain variable domain, and the second CID domain comprises a heavy chain variable domain and a light chain variable domain capable of binding to the complex formed between the first CID domain and the CID small molecule.

In some embodiments, the CID small molecule is methotrexate.

In some embodiments, the first CID domain is BCL-2 or variants thereof, the CID small molecule is ABT-199 or ABT-263, and the second CID domain comprises a heavy chain variable domain and a light chain variable domain capable of binding to the complex formed between the first CID domain and the CID small molecule. In some embodiments, the first CID domain is BCL-2 or BCL-2 (C158A), the CID small molecule is ABT-199, and the second CID domain comprises a variable heavy domain (VH) comprising a vhCDR1 comprising SEQ ID NO:1, a vhCDR2 comprising SEQ ID NO:72, a vhCDR3 comprising SEQ ID NO:129; and a variable light domain (VL) comprising a vlCDR1 comprising SEQ ID NO:310, a vlCDR2 comprising SEQ ID NO:311, and a vlCDR3 comprising SEQ ID NO:233.

In some embodiments, the first CID domain is an ABT-737 binding domain of Bcl-xL, the CID small molecule is ABT-737, and the second CID domain comprises a heavy chain variable domain and a light chain variable domain capable of binding to the complex formed between said first CID domain and said CID small molecule.

In some embodiments, the first CID domain is an rapamycin binding domain of FKBP, the CID small molecule is rapamycin, and the second CID domain comprises a heavy chain variable domain and a light chain variable domain capable of binding to the complex formed between the first CID domain and the CID small molecule.

In some embodiments, the first CID domain is an GDC-0152, LCL161, AT406, CUDC-427, or Birinapant binding domain of cIAPl, the CID small molecule is GDC-0152, LCL161, AT406, CUDC-427, or Birinapant, and the second CID domain comprises a heavy chain variable domain and a light chain variable domain capable of binding to the complex formed between the first CID domain and the CID small molecule.

In some embodiments, the first CID domain is thalidomide binding domain of cereblon, the small molecule is thalidomide, lenalidomide, or pomalidomide, and the second CID domain comprises a heavy chain variable domain and a light chain variable domain capable of binding to the complex formed between the first CID domain and the CID small molecule.

In some embodiments, the therapeutic moiety is selected from an antibody, an antibody fragment, a cytokine, a hormone, a peptide, and an antibody drug conjugate. In some embodiments, the therapeutic moiety is a bispecific antibody.

In some embodiments, the therapeutic moiety is a bispecific T cell engager moiety. In some embodiments, the bispecific T cell engager moiety comprises a T cell antigen-binding domain and a tumor-associated antigen-binding domain. In some embodiments, the T cell antigen is CD3 and the tumor-associated antigen is CD19.

In some embodiments, the therapeutic moiety is a human interleukin molecule. In some embodiments, the therapeutic moiety is human IL-2.

In some embodiments, the first CID domain is linked to the first Fc domain via a first linker. In some embodiments, the second CID domain is linked to the therapeutic moiety via a second linker.

In some embodiments, the IgG is human IgG1.

In some embodiments, the first Fc domain is a first variant Fc domain, and the second Fc domain is a second variant Fc domain.

Another aspect of the invention relates to a method of extending serum half-life of a therapeutic moiety in a patient. The method comprises administering to the patient (1) the composition including its various embodiments described above; and (2) the CID small molecule described above. Adminstration of the small molecule induces the first and second CID domains to form a complex, thereby extending serum half-life of the therapeutic moiety.

Another aspect of the invention relates to a method of clearing a therapeutic moiety from a patient who has been administered a composition comprising the therapeutic moiety, and a CID small molecule described above. The method comprises ceasing administration of the CID small molecule to the patient, such that the therapeutic moiety is cleared from said patient’s blood.

Another aspect of the invention relates to a composition comprising (1) a heterodimeric Fc fusion protein comprising (a) a first monomer comprising a first chemically inhibited dimerizer (CInD) domain and a first Fc domain of IgG, wherein the first CInD domain is covalently linked to the first Fc domain, and (b) a second monomer comprising a second Fc domain of IgG; and (2) a fusion protein moiety comprising a second CInD domain and a second therapeutic moiety, wherein the second CInD domain is covalently linked to the second therapeutic moiety at N or C terminus. The first CInD domain binds to the second CInD domain and forms a complex, and the complex can be disrupted by a CInD small molecule.

In some embodiments, the first CInD domain or the second CInD domain comprises an antibody moiety.

In some embodiments, the first CInD domain is linked to the first Fc domain via a first linker. In some embodiments, the second CInD domain is linked to the therapeutic moiety via a second linker.

In some embodiments, wherein the above mentioned IgG is human IgG1.

In some embodiments, the first Fc domain is a first variant Fc domain, and the second Fc domain is a second variant Fc domain.

In some embodiments, the second therapeutic moiety is selected from an antibody, an antibody fragment, a cytokine, a hormone, a polypeptide, and an antibody drug conjugate. In some embodiments, the second therapeutic moiety is a bispecific antibody. In some embodiments, the second therapeutic moiety is a bispecific T cell engager moiety. In some embodiments, the second therapeutic moiety is a bispecific T cell engager moiety comprising a T cell antigen-binding domain and a tumor-associated antigen-binding domain. In some embodiments, the T cell antigen is CD3 and the tumor-associated antigen is CD19.

In some embodiments, the second therapeutic moiety is a human interleukin molecule. In some embodiments, the second therapeutic moiety is human IL-2.

In some embodiments, the above described second monomer further comprises a first therapeutic moiety covalently linked to the second Fc domain. In some embodiments, the first therapeutic moiety is selected from an antibody, an antibody fragment, a cytokine, a hormone, a polypeptide, and an antibody drug conjugate.

In some embodiments, the first therapeutic moiety is a T cell antigen-binding domain and the second therapeutic moiety is a tumor-associated antigen-binding domain. Alternatively, the first therapeutic moiety is a tumor-associated antigen-binding domain and the second therapeutic moiety is a T cell antigen-binding domain. In some embodiments, the T cell antigen is CD3 and the tumor-associated antigen is CD19.

Another aspect of the invention relates to a composition comprising (1) a homodimeric Fc fusion protein comprising two identical monomers, wherein the two monomers each comprising a first CInD domain covalently linked to a Fc domain of IgG; and (2) a fusion protein moiety comprising a second CInD domain and a therapeutic moiety, wherein the second CInD domain is covalently linked to the therapeutic moiety at N or C terminus. The first CInD domain binds to the second CInD domain forming a complex, and the complex can be disrupted by a CInD small molecule.

In some embodiments, either the first CInD domain or the second CInD domain comprises an antibody moiety.

In some embodiments, the first CInD domain is linked to the first Fc domain via a first linker. In some embodiments, the second CInD domain is linked to the therapeutic moiety via a second linker.

In some embodiments, the IgG is a human IgG1.

In some embodiments, the therapeutic moiety is selected from an antibody, an antibody fragment, a cytokine, a hormone, a polypeptide, and an antibody drug conjugate. In some embodiments, the therapeutic moiety is a bispecific antibody. In some embodiments, the therapeutic moiety is a bispecific T cell engager moiety. In some embodiments, the bispecific T cell engager moiety comprises a T cell antigen-binding domain and a tumor-associated antigen-binding domain. In some embodiments, the T cell antigen is CD3 and the tumor-associated antigen is CD19.

In some embodiments, the therapeutic moiety is a human interleukin molecule. In some embodiments, the therapeutic moiety is human IL-2.

Another aspect of the invention relates to a method of extending serum half-life of a therapeutic moiety in a patient, and the method comprises administering to the patient any of the composition comprising a CInD as decribed above.

Another aspect of the invention relates to a method of clearing a therapeutic moiety from a patient who has been previously administered the composition comprising a CInD as decribed above. The method comprises administering the CInD small molecule to the patient, dissociating the therapeutic moiety from the heterodimeric or homodimeric Fc fusion protein.

The present invention provides a tunable control system for serum half-life of a T cell engager. In one aspect, the present invention provides a composition, which comprises a first monomer and a second monomer, wherein the first monomer includes a first CID domain, an optional domain linker, and a human serum albumin (HSA) binding domain; the second monomer includes a second CID domain, an optional domain linker and a T cell engager; and the first domain and second domain associate in the presence of a CID small molecule to form the first domain-CID small molecule-second domain complex. In the absence of the small molecule, the first domain and second domain do not associate with each other. The T cell engager comprises a CD3 antigen binding domain (ABD), an optional domain linker and a tumor-associated antigen (TAA) binding domain.

In some embodiments, the small molecule is selected from the group consisting of FK1012, rimiducid, FK506, FKCsA, Rapamycin, Rapamycin analogs, Courmermycin, Gibberellin, HaXS, TMP-tag, ABT-737.

In some embodiments, the complex of the first domain-CID small molecule-second domain is selected from the group consisting of FKBP-FK1012-FKBP, variant FKBP-rimiducid-variant FKBP, FKBP-FK506-Calcineurin, FKBP-FKCsA-CyP-Fas, FKBP-Rapamycin-FRB, variant FKBP-Rapamycin analogs-variant FRB, GyrB-Courmermycin-GyrB, GAI-Gibberellin-GID1, SNAP-tag-HaXS-HaloTag, eDHFR-TMP-tag-HaloTag, AZ1-ABT-737-BCL-xL, Calcineurin-FK506-FKBP, CyP-Fas-FKCsA-FKBP, FRB-Rapamycin-FKBP, variant FRB-Rapamycin analogs-variant FKBP, GID1-Gibberellin-GAI, HaloTag-HaXS-SNAP-tag, HaloTag-TMP-tag-eDHFR, BCL-xL-ABT-737-AZ1.

In some embodiments, the HSA binding domain comprises a heavy chain variable domain and a light chain variable domain.

In some embodiments, the first domain is linked to the HSA binding domain via a first linker, and the second domain is linked to the T cell engager via a second linker.

In another aspect, the present invention provides a pharmaceutical composition comprising any one of the compositions described above.

In another aspect, the present invention provides a method of extending serum half-life of a T cell engager in a patient, and the method comprises administering to the patient any one of the compositions or pharmaceutical compositions as described herein, and administering to the patient a CID small molecule which induces association of the first CID and second CID domain described herein, thereby extending serum half-life of the T cell engager.

In another aspect, the present invention provides a method of treating cancer in a patient, and the method comprises administering to the patient any one of the compositions or pharmaceutical compositions as described herein, and administering to the patient a CID small molecule which induces association of the first CID and second CID domain described herein, thereby treating cancer.

In another aspect, the present invention provides a method of clearing a T cell engager from a patient who has been administered a composition containing the T cell engager and a CID small molecule as described herein, and the method comprises stopping administration of the small molecule to the patient, so that the T cell engager no longer associates with HSA and is cleared from the patient’s blood.

In another aspect, the present invention provides a method of treating cancer in a patient, the method comprising: a) administering to said patient said composition or said pharmaceutical composition comprising said T cell engager according to any one of the compositions described herein; b) administering to said patient said small molecule according to any of the compositions described herein; wherein said first and second CID domains form complex with said small molecule in said patient to treat cancer.

The present invention also provides a tunable control system for serum half-life of a T cell engager. In one aspect, the present invention provides a composition, which comprises a first monomer and a second monomer, wherein the first monomer includes a first CID domain, an optional domain linker, and a human serum albumin (HSA) binding domain; the second monomer includes a second CID domain, an optional domain linker and a T cell engager; and the first domain and second domain associate in the presence of a CID small molecule to form the first domain-CID small molecule-second domain complex. In the absence of the small molecule, the first domain and second domain do not associate with each other. The T cell engager comprises a CD3 antigen binding domain (ABD), an optional domain linker and a tumor-associated antigen (TAA) binding domain.

In some embodiments, the small molecule is selected from the group consisting of FK1012, rimiducid, FK506, FKCsA, Rapamycin, Rapamycin analogs, Courmermycin, Gibberellin, HaXS, TMP-tag, ABT-737.

In some embodiments, the complex of the first domain-CID small molecule-second domain is selected from the group consisting of FKBP-FK1012-FKBP, variant FKBP-rimiducid-variant FKBP, FKBP-FK506-Calcineurin, FKBP-FKCsA-CyP-Fas, FKBP-Rapamycin-FRB, variant FKBP-Rapamycin analogs-variant FRB, GyrB-Courmermycin-GyrB, GAI-Gibberellin-GID1, SNAP-tag-HaXS-HaloTag, eDHFR-TMP-tag-HaloTag, AZ1-ABT-737-BCL-xL, Calcineurin-FK506-FKBP, CyP-Fas-FKCsA-FKBP, FRB-Rapamycin-FKBP, variant FRB-Rapamycin analogs-variant FKBP, GID1-Gibberellin-GAI, HaloTag-HaXS-SNAP-tag, HaloTag-TMP-tag-eDHFR, BCL-xL-ABT-737-AZ1.

In some embodiments, the HSA binding domain comprises a heavy chain variable domain and a light chain variable domain.

In some embodiments, the first domain is linked to the HSA binding domain via a first linker, and the second domain is linked to the T cell engager via a second linker.

In another aspect, the present invention provides a pharmaceutical composition comprising any one of the compositions described above.

In another aspect, the present invention provides a method of extending serum half-life of a T cell engager in a patient, and the method comprises administering to the patient any one of the compositions or pharmaceutical compositions as described herein, and administering to the patient a CID small molecule which induces association of the first CID and second CID domain described herein, thereby extending serum half-life of the T cell engager.

In another aspect, the present invention provides a method of treating cancer in a patient, and the method comprises administering to the patient any one of the compositions or pharmaceutical compositions as described herein, and administering to the patient a CID small molecule which induces association of the first CID and second CID domain described herein, thereby treating cancer.

In another aspect, the present invention provides a method of clearing a T cell engager from a patient who has been administered a composition containing the T cell engager and a CID small molecule as described herein, and the method comprises stopping administration of the small molecule to the patient, so that the T cell engager no longer associates with HSA and is cleared from the patient’s blood.

### BRIEF DESCRIPTION OF THE DRAWINGS

FIGS. 1A-1B depicts one aspect of the present invention. Generally, a heterodimeric Fc fusion protein (101) and a fusion protein moiety (102) are co-administered to a patient. The heterodimeric Fc fusion protein (101) comprises a first monomer containing a first CID domain (103) as defined herein linked using a domain linker (104) to a first Fc domain (105) (e.g., human IgG1 Fc) and a second monomer containing a second Fc domain (106) that heterodimerizes with the first Fc domain. The fusion protein (102) (which is essentially a third monomer) comprises a second CID domain (107) defined herein linked using a domain linker (108) to a therapeutic moiety (109). Upon exposure to the CID small molecule (110) (e.g., when the CID small molecule is administered to the patient), the first CID domain (103) and second CID domain (107) each associate with the CID small molecule (110) such that a complex of the heterodimeric Fc fusion protein (101) and the fusion protein moiety (102) is formed. Thus, the therapeutic moiety is now non-covalently associated with an Fc domain, and the entire complex is protected from rapid clearance from the patient’s bloodstream and the serum half-life of the therapeutic moiety (109) is extended. Generally, as the CID small molecule has a very short half-life in serum, the administration of the CID small molecule will continue over time. At some point, when the activity of the therapeutic moiety is either no longer required or is resulting in adverse side effects, the administration of the CID small molecule is stopped, resulting in complex disassociation, followed by clearance of the fusion protein moiety (102) from the patient. In some embodiments as shown in FIG. 1B, the therapeutic moiety (109) is a T cell engager comprising an antigen binding domain (ABD) (112) (e.g. an anti-CD19 ABD in the form of a scFv) linked to an anti-CD3 antigen binding domain (111) in the form of scFv.

FIGS. 2A-2B depicts another aspect of the present invention. Generally, a heterodimeric Fc fusion protein (101) and a fusion protein moiety (102) are co-administered to a patient. The heterodimeric Fc fusion protein (101) comprises a first monomer containing a first CInD domain (104) as defined herein linked using a domain linker (103) to a first Fc domain (105) (e.g., human IgG1 Fc) and a second monomer containing a second Fc domain (106) that heterodimerizes with the first Fc domain. The fusion protein moiety comprises a second CInD domain (107) defined herein linked using a domain linker (108) to a therapeutic moiety (109). Upon co-administration, the first CID domain (105) and second CInD domain (106) associate to form a complex. Thus, the therapeutic moiety (109) is now non-covalently associated with an Fc domain, and the entire complex is protected from rapid clearance from the patient’s bloodstream and the serum half-life of the therapeutic moiety (109) is extended. At some point, when the activity of the therapeutic moiety (109) is either no longer required or is resulting in adverse side effects, a CInD small molecule (110) is administered to the patient disrupting the complex formed between the first CID domain (105) and second CInD domains (106). As a result, the therapeutic moiety (109) dissociates from the Fc fusion protein and is rapidly cleared from serum due to its short serum half-life. In some embodiments as shown in FIG. 1B, the therapeutic moiety is a T cell engager comprising an anti-CD3 antigen binding domain (ABD) (111) linked to an antigen binding domain (e.g., an anti-CD19 antigen binding domain 112) in the form of scFv.

FIG. 3 depicts another aspect of the present invention. Generally, a heterodimeric Fc fusion protein (101) and a fusion protein moiety (102) are co-administered to a patient. The heterodimeric Fc fusion protein (101) comprises a first monomer containing a first CInD domain (104) as defined herein linked using a domain linker (103) to a first Fc domain (e.g., human IgG1 Fc) (105), and a second monomer containing a second Fc domain (106) that is covalently linked to a first therapeutic moiety (111) and heterodimerizes with the first Fc domain (105). The fusion protein moiety (102) comprises a second CInD domain (107) defined herein linked using a domain linker (108) to a second therapeutic moiety (109). Upon co-administration, the first CID domain (107) and the second CInD domain (107) associate to form a complex, thus, bring two therapeutic moieties (111) and (109) together (e.g., an anti-CD3 antigen binding domain and a tumor-associated antigen binding domain such as an anti-CD19 antigen binding domain). The second therapeutic moiety (109) is now non-covalently associated with an Fc domain, and is protected from rapid clearance from the patient’s bloodstream. The serum half-life of the second therapeutic moiety (109) is extended. At some point, when the activity of the second therapeutic moiety (109) is either no longer required or is resulting in adverse side effects, a CInD small molecule (110) is administered to the patient disrupting the complex formed between the first CID domain (104) and the second CInD domain (107). As a result, the second therapeutic moiety 109 dissociates from the Fc fusion protein and is rapidly cleared from serum.

FIG. 4 depicts another aspect of the present invention. Generally, a homodimeric Fc fusion protein (101) and a fusion protein moiety (102) are co-administered to a patient. The homodimeric Fc fusion protein (101) comprises two identical monomers each containing a first CInD domain (104) as defined herein linked using a domain linker (103) to a first Fc domain (105) (e.g., human IgG1 Fc). The fusion protein moiety (102) comprises a second CInD domain (106) defined herein linked using a domain linker (107) to a therapeutic moiety (108). Upon co-administration, the first CID domain (104) and second CInD domain (106) associate to form a complex, thus, bring two therapeutic moieties together. The therapeutic moieties (108) are now non-covalently associated with an Fc domain, and are protected from rapid clearance from the patient’s bloodstream. The serum half-life of the therapeutic moieties (108) is extended. At some point, when the activity of the therapeutic moiety (108) is either no longer required or is resulting in adverse side effects, a CInD small molecule (109) is administered to the patient disrupting the complex formed between the first CID domain (104) and second CInD domain (106). As a result, the therapeutic moieties (108) dissociate from the Fc fusion protein and are rapidly cleared from serum.

FIG. 5 illustrates one example of a heterodimeric Fc fusion protein Ab59 which comprises a first monomer containing a first CID domain (BCl-2 C158A) linked using a domain linker to a first Fc domain (human IgG1 Fc), and a second monomer containing a second Fc domain (human IgG1 Fc) that heterodimerizes with the first Fc domain.

FIG. 6 illustrates exemplary fusion protein moieties, each of which comprises a second CID domain (AZ-21) and a T cell engager containing an anti-CD19 antigen binding domain linked to an anti-CD3 antigen binding domain in the form of scFv. AZ-21 can be linked to the N or C terminus of the T cell engager. AZ-21 can be made in a Fab or single chain Fab format. An anti-CD3 antigen binding domain can be derived from the clone L2K or UCHT1.v9. His tag is used to facilitate purification of the fusion protein moiety.

FIGS. 7A-7J show amino acid sequences of the exemplary fusion protein moieties shown in FIGS. 5 and 6.

FIG. 8A illustrates exemplary fusion protein moieties, each of which comprises a second CID domain (AZ-21) linked to human IL-2 (hIL-2) via a domain linker. AZ-21 is in the format of scFv, and can be linked to the N or C terminus of hIL-2. His tag is used to facilitate purification of the fusion protein moiety. FIG. 8B provides amino acid sequences of IL-2, IL-12 and IL-15 and variants thereof.

FIG. 9 shows amino acid sequences of the exemplary fusion protein moieties shown in FIG. 8.

FIG. 10 shows the amino acid sequences of AZ-21, BCL-2 and BCL-2 (C158A). AZ-21 and BCL-2 or BCL-2 (C158A) form CID in the presence of a CID small molecule ABT-199.

FIG. 11 shows the amino acid sequences of vh-CDRs and vl-CDRs of a second CID domain, which is capable of forming a complex with the first CID domain Bcl-xL in the presence of the CID small molecule ABT-737. Each clone represent is a second CID domain.

FIGS. 12A and 12B show the amino acid sequences of vh-CDRs and vl-CDRs of a second CID domain, which is capable of forming a complex with the first CID domain BCL-2 or BCL-2 (C158A) in the presence of the CID small molecule ABT-199 (venetoclax). Each clone represent is a second CID domain.

FIG. 13 shows the amino acid sequences of vh-CDRs and vl-CDRs of a second CID domain, which is capable of forming a complex with the first CID domain BCL-2 in the presence of the CID small molecule ABT-263. Each clone represent is a second CID domain.

FIG. 14 shows the amino acid sequences of vh-CDRs and vl-CDRs of a second CID domain, which is capable of forming a complex with the first CID domain cIAPl in the presence of the CID small molecule LCL161. Each clone represent is a second CID domain.

FIG. 15 shows the amino acid sequences of vh-CDRs and vl-CDRs of a second CID domain, which is capable of forming a complex with the first CID domain cIAPl in the presence of the CID small molecule GDC-0152. Each clone represent is a second CID domain.

FIG. 16 shows the amino acid sequences of vh-CDRs and vl-CDRs of a second CID domain, which is capable of forming a complex with the first CID domain cIAPl in the presence of the CID small molecule AT406. Each clone represent is a second CID domain.

FIG. 17 shows the amino acid sequences of vh-CDRs and vl-CDRs of a second CID domain, which is capable of forming a complex with the first CID domain cIAPl in the presence of the CID small molecule CUDC-427. Each clone represent is a second CID domain.

FIG. 18 shows the amino acid sequences of vh-CDRs and vl-CDRs of a second CID domain, which is capable of forming a complex with the first CID domain FKBP in the presence of the CID small molecule rapamycin. Each clone represent is a second CID domain.

FIG. 19 shows the amino acid sequences of vh-CDRs and vl-CDRs of a second CID domain, which is capable of forming a complex with the first CID domain - a methotrexate binding domain - in the presence of the CID small molecule methotrexate.

FIGS. 20A-20B shows dose-response curves of Jurkat T cell activation incubated with Ab52, Ab53, Ab54, Ab55, Ab57 and Ab63.

FIG. 21 shows dose-response curves of Jurkat T cell activation incubated with Ab52, Ab53, Ab54, Ab55, and Ab57 in the presence of Ab59 and ABT-199 or vehicle control.

FIG. 22A shows dose-response curves of Raji cell cytotoxicity after co-culture with primary human T cells and Ab53 or Ab57. FIG. 22B shows dose-response curves of Raji cell cytotoxicity after co-culture with primary human T cells and Ab53 in the presence of Ab59 and ABT-199 or vehicle control.

FIG. 23 shows phosphorylation of STAT5 detected in human T cells, wherein the human T cells were treated with hIL-2 or a fusion protein moiety comprising hIL-2.

FIG. 24 shows size-exclusion chromatogram of fusion protein moieties.

FIG. 25A shows biolayer interferometry of Ab53 and Ab57 binding to immobilized BCL-2. FIG. 25B shows biolayer interferometry of Ab59 binding to immobilized AZ21.

FIG. 26 shows biolayer interferometry of Ab93 and Ab94 binding to immobilized BCL-2 in the presence or absence of ABT-199. KD values for binding are shown.

FIG. 27 shows amino acid sequences of exemplary anti-CD3 ABD.

FIG. 28 shows amino acid sequences of exemplary IgG1 Fc domains.

FIG. 29 depicts another aspect of the present invention. Generally, there are two monomers that are co-administered to a patient: a first monomer that comprises a first CID domain as defined herein linked using a domain linker to a human serum albumin (HSA) binding domain. Upon administration to the patient, the first monomer associates with HSA in the blood stream of the patient. The second monomer comprises a second CID domain linked using a domain linker to a T cell engager domain as defined herein. Upon exposure to the CID small molecule (e.g., when the CID small molecule is administered to the patient), the first and second CID domains each associate with the CID small molecule such that a dimer of the two monomers is formed. Thus, the T cell engager domain is now non-covalently associated with HSA, and the entire complex is protected from rapid clearance from the patient’s bloodstream, and will circulate and result in T cell engagement with a tumor cell, resulting in treatment of the cancer. Thus, co-administration of the first and second monomer and the CID small molecule results in treatment of cancer. Generally, as the CID small molecule has a very short half-life in serum, the administration of the CID small molecule will continue. At some point, when T cell engaging activity is either no longer required or is resulting in adverse side effects, the administration of the CID small molecule is stopped, resulting in dimer disassociation, followed by the clearance of the second monomer with the T cell engager domain from the patient.

FIG. 30A shows amino acid sequence of a monomer comprised of a first CID domain linked to an HSA ABD [Bcl-2(C1158A) linked to single domain anti-HSA antibody].

FIG. 30B shows binding curve of the above monomer to the second CID domain AZ21 in the presence of the CID small molecule ABT-199.

FIGS. 31A and 31B show amino acid sequences of exemplary HSA binding domains.

FIG. 32 shows chemically induced dimerization enabled small-molecule control over the half-life of a bispecific T-cell engager. (A) Schematic of CID-based half-life extension of a bispecific T-cell engager. (B) The Therapeutic Module and Half-life Extension Module used in this study. (C) The plasma elimination half-life in mice of bispecific Ab57 was extended by 5-fold when mice were dosed with venetoclax. ****: P ≤ 0.0001, 2 tailed t-test.

FIG. 33 shows chemically induced dimerization enabled small-molecule control over the half-life of IL-2. (A) Schematic of CID-based half-life extension of IL-2. (B) The Therapeutic Module and Half-life Extension Module used in this study. (C) The plasma elimination half-life in mice of cytokine IL-2 was extended by 17-fold when mice were dosed with venetoclax. ****: P ≤ 0.0001, 2 tailed t-test.

### DETAILED DESCRIPTION OF THE INVENTION

### A. Overview

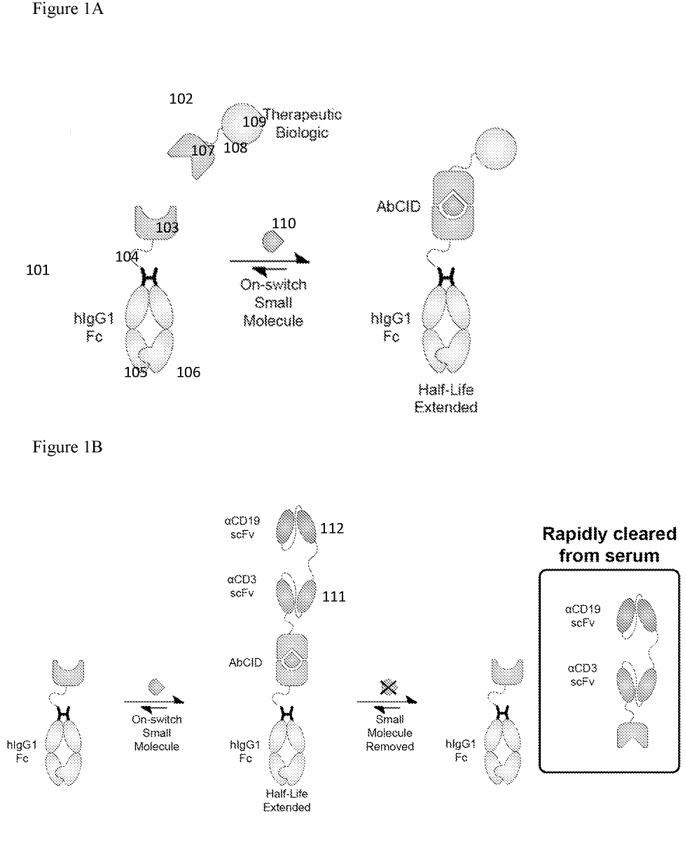

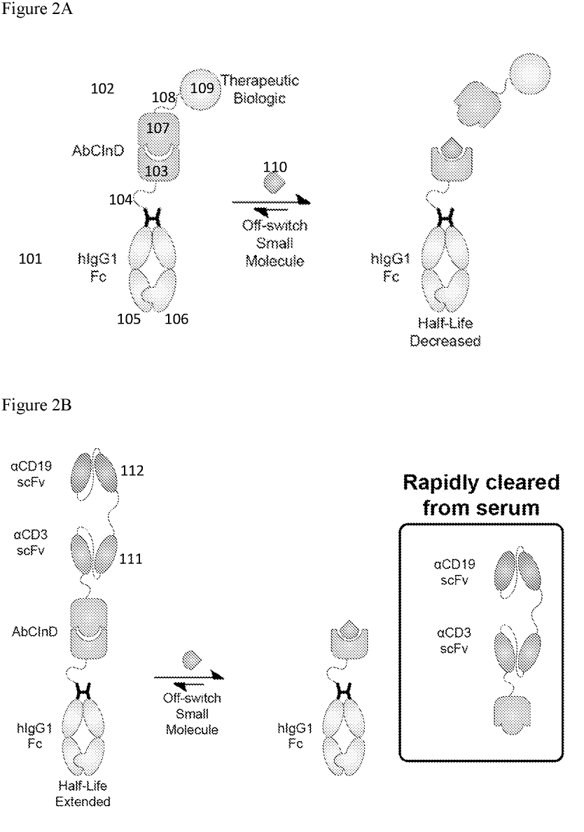

The present invention enables tunable control of a therapeutic moiety’s half-life in serum through addition of a small molecule (e.g. administration to the patient) or removal of a small molecule (e.g. the cessation of administration, followed by clearance of the small molecule by the patient) that engage chemically induced dimerization (CID) domains as generally outlined in the figures. These small molecules are either a chemically induced dimerizer (CID) small molecule (CIDSM), that induces the formation of a dimer such as depicted in FIG. 1, or a chemically inhibited dimerizer (CInD) small molecule (CInDSM), that disrupts the dimer as depicted in FIG. 2.

In general, the present invention is directed to extending the serum half life of therapeutic molecules by associating the molecules with a half-life extension moiety, such as an Fc domain or human serum albumin (HSA). As is known in the art, association of a biologic drug that is generally rapidly cleared from the body with either an Fc domain or HSA results in the extension of the half-life of the drug in serum.

Accordingly, relating to the use of an Fc domain as the half-life extension molecule is generally depicted in FIG. 1A, one aspect of the invention involves linking a Fc domain to one half of a chemically induced dimerizer (CID), referred herein as “a first CID domain”, optionally via a domain linker. A therapeutic moiety is linked to the other half of the CID, referred herein as “a second CID domain”, optionally via a domain linker. Thus the compositions of FIG. 1 generally have three protein chains, or monomers: the first monomer comprising the first Fc domain, a domain linker and the first CID domain; a second monomer comprising the second Fc domain, and a third monomer, also referred to herein as a fusion protein moiety. Addition of a CIDSM induces association of the two halves of the CID, thereby enabling association of the therapeutic moiety with the Fc domain, and extending the serum half-life of the therapeutic moiety. In the event that the therapeutic moiety needs to be cleared from the blood quickly, the administration of the CID small molecule is ceased, leading to dissociation of the two halves of the CID and the dissociation of the therapeutic moiety from the Fc domain.

For example, a patient can be dosed with a composition comprising a Fc fusion protein and a fusion protein moiety as described herein. The patient can also be administered a CID small molecule that induces dimerization of the two halves of the CID, thus bringing the Fc fusion protein and a fusion protein moiety together to form a dimer. As a result, the therapeutic moiety immediately associates with the Fc domain and its serum half-life is extended. To maintain association of the therapeutic moiety with the Fc domain, the patient can be dosed regularly with the CIDSM, wherein the frequency of dosing depends on a combination of the CIDSM’s serum half-life, the binding affinity of CIDSM to the first and second CID domains, and the lifetime of the CID complex (e.g. the CID dimer). In the event that the patient needs to have the therapeutic moiety cleared quickly, for example, due to safety concerns, the patient would stop being dosed with the CIDSM, leading to clearance of the CIDSM in the patient, disassociation of the therapeutic moiety from the Fc domain, and clearance of the therapeutic moiety in the patient. The rate of clearance of the therapeutic moiety depends on a combination of the CIDSM’s serum half-life, the lifetime of the CID complex, and the clearance rate of the therapeutic moiety which is no longer associated with the Fc domain.

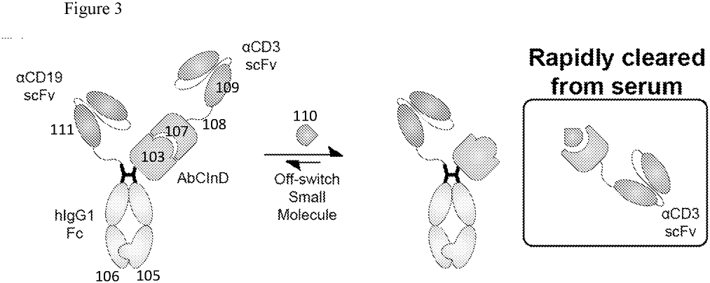

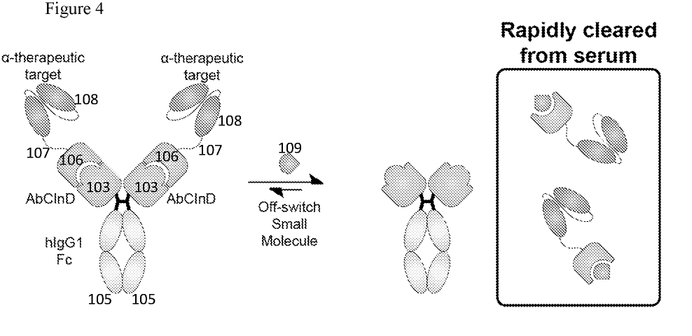

As generally depicted in FIG. 2A, FIG. 3 and FIG. 4, another aspect of the invention involves linking a first Fc domain to one half of a chemically inhibited dimerizer (CInD), referred herein as “a first CInD domain”, optionally via a domain linker. The second monomer is the second Fc domain, which forms the heterodimeric Fc domain together with first Fc domain. The third monomer (also referred to herein as a fusion protein moiety) comprises a therapeutic moiety linked to the other half of the CInD, referred herein as “a second CInD domain”, optionally via a domain linker. The two halves of CInD domain associate with each other and form a dimer, enabling association of the therapeutic moiety with the heterodimeric Fc domain, and extending the serum half-life of the therapeutic moiety. In the event that the therapeutic moiety needs to be cleared from the blood quickly, a CInD small molecule is administered, which induces disassociation of the two halves of the CInD, thereby enabling disassociation of the therapeutic moiety from the heterodimeric Fc domain, and clearance of the therapeutic moiety in the patient.

One of skill in the art will appreciate that the term “dimer” is used in two contexts herein. One context refers to the first and second Fc domains coming together to form a dimer (a heterodimeric Fc domain in the case of FIGS. 1, 2 and 3, for example, and a homodimeric Fc domain in the case of FIG. 4, for example). The second context refers to the dimers formed by using CIDSM of the invention that brings together the first and second CID domains of the invention, and the dimers formed between the first and second CInD domains domains of the invention.

In some embodiments as generally depicted in FIG. 2A, the Fc fusion protein is heterodimeric with one monomer containing a first CInD domain linked to a first Fc domain, and the other monomer containing a second Fc domain alone (e.g. an “empty Fc domain”). The first and second Fc domain heterodimerize, for example, by incorporating the heterodimerization mutations described herein. The third monomer, the fusion protein moiety, comprises a second CInD domain linked via a domain linker to a therapeutic moiety as described herein.

In some embodiments as generally depicted in FIG. 3, the Fc fusion protein is heterodimeric with one monomer containing a first CInD domain linked to a first Fc domain. The other Fc monomer contains a second Fc domain linked to a first therapeutic moiety. The third monomer comprises the second CInD domain linked with a domain linker to a second therapeutic moiety. The first and second Fc domains heterodimerize, for example, by incorporating the heterodimerization mutations described herein. Administration of the fusion protein moiety comprising the second therapeutic moiety linked to the second CInD domain induces the association of the two halves of the CInD domains, enabling association of the second therapeutic moiety with the Fc domain, and extending its serum half-life. In addition, this format imparts a bispecific nature to the therapeutic moiety and the second therapeutic moiety while simultaneously increasing their half-life.

In some other embodiments as depicted in FIG. 4, the Fc fusion protein is homodimeric with two identical monomers, each containing a first CInD domain linked to an Fc domain, optionally via a domain linker. Administration of a fusion protein moiety comprising a therapeutic moiety linked to a second CInD domain induces the association of the two halves of the CInD domains, enabling association of the two therapeutic moieties with a single Fc dimer, and extending the serum half-life of the therapeutic moieties. This format increases the stoichiometry and valancing of the therapeutic moieties while simultaneously extending their half-life.

In some embodiments, administration of the invented compositions described herein extends the serum half-life of therapeutic moieties to at least about 2 days, at least about 4, at least about 6, at least about 8 days, at least about 10 days, at least about 12 days, or at least about 14 days. This contrasts to administration of the therapeutic moieties alone which have relatively much shorter serum elimination half-life. When the therapeutic moieties are no longer needed, they can be rapidly removed by removal (e.g., the cessation of administration, followed by clearance of the CID small molecule by the patient) of a short-lived CID small molecule or addition (e.g. administration to the patient) of the CInD small molecule.

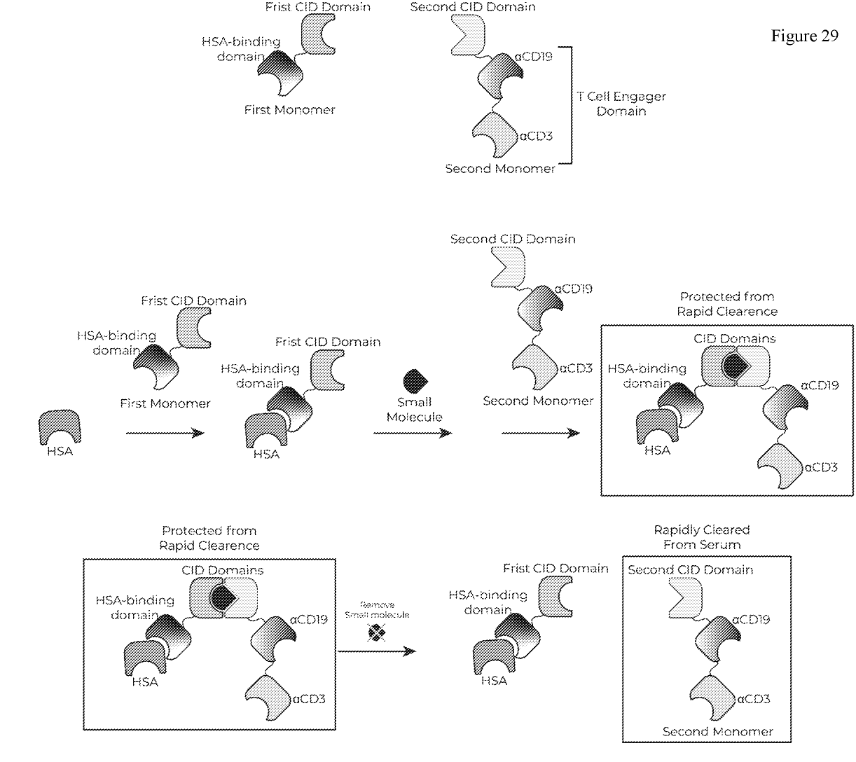

As it relates to the use of human serum albumin (HSA) as the half life extension moiety, this is generally depicted in FIG. 29. As generally depicted in FIG. 29, The T cell engager domain, is linked to one half of a chemically induced dimerizer (CID), referred herein as “a first CID domain”, optionally via a domain linker. A human serum albumin (HSA) binding domain is linked to the other half of the CID, referred herein as “a second CID domain”, optionally via a domain linker. The HSA binding domain can constitutively bind to HSA. Addition of a small molecule induces association of the two halves of the CID, thereby bringing together the two monomers and enabling association of the T cell engager domain with HSA, and extension of the serum half-life of the T cell engager domain. In the event that the T cell engager domain needs to be cleared from the blood quickly, the administration of the CID small molecule is ceased, leading to dissociation of the two halves of the CID and the dissociation of the T cell engager domain from HSA.

For example, a patient can be dosed with a composition comprising a first and second monomer as described herein. The patient can also be administered a CID small molecule that induces dimerization of the two halves of the CID, thus bringing the two monomers together to form a dimer. As a result, the T cell engager domain immediately associates with HSA and its serum half-life is extended. To maintain association of the T cell engager domain with HSA, the patient can be dosed regularly with the CID small-molecule, wherein the frequency of dosing depends on a combination of the CID small molecule’s serum half-life, the binding affinity of the CID small molecule to the first and second CID domains, and the lifetime of the CID complex (e.g. the CID dimer). In the event that the patient needs to have the T cell engager cleared quickly, for example, due to safety concerns, the patient would stop being dosed with the small molecule, leading to clearance of the small molecule in the patient, disassociation of the T cell engager from HSA, and clearance of the T cell engager in the patient. The rate of clearance of the T cell engager depends on a combination of the small molecule drug’s serum half-life, the lifetime of the CID complex, and the clearance rate of the T cell engager which is no longer associated with HSA.

In some embodiments, administration of the composition described herein extends the serum elimination half-life of the T cell engager to at least about 2 days, at least about 4, at least about 6, at least about 8 days, at least about 10 days, at least about 12 days, or at least about 14 days. This contrasts to administration of the T cell engager alone which have relatively much shorter serum elimination half-life. For example, Blincyto®, a CD19×CD3 bispecific scFv-scFv fusion molecule requires continuous intravenous infusion due to its short elimination serum half-life.

Administration of the composition described herein can not only extend the serum half-life of the T cell engager, but also enable rapid removal of the T cell engager when it is not needed by stopping the dosing of the small molecule.

### B. Definitions

In order that the application may be more completely understood, several definitions are set forth below. Such definitions are meant to encompass grammatical equivalents.

Unless explained otherwise, all technical and scientific terms used herein have the same meaning as commonly understood to one of ordinary skill in the art to which this disclosure belongs.

Accession Numbers: Reference numbers assigned to various nucleic acid and amino acid sequences in the NCBI database (National Center for Biotechnology Information) that is maintained by the National Institute of Health, U.S.A. The accession numbers listed in this specification are herein incorporated by reference as provided in the database as of the date of filing this application.

The term “antigen binding domain” or “ABD” herein is meant a set of six Complementary Determining Regions (CDRs) that, when present as part of a polypeptide sequence, specifically binds a target antigen as discussed herein. Thus, an “HSA antigen binding domain” binds human serum albumin as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light chain. The CDRs are present in the variable heavy and variable light domains, respectively, and together form an Fv region. Thus, in some cases, the six CDRs of the antigen binding domain are contributed by a variable heavy and variable light chain. For example, in a scFv format, the vh and vl domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used). In some cases, the linker is a domain linker as described herein.

Additionally, in some cases, an ABD used in the invention can be a single domain ABD (“sdABD”). By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). These are sometimes referred to in the art as “VHH” domains.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., SEQUENCES OF PROTEINS OF IMMUNOLOGICAL INTEREST, 5th Ed. Public Health Service, National Institutes of Health, Bethesda, Md. (1991)).

Table 1

| | Kabat+ Chothia | IMGT | Kabat | AbM | Chothia | Contact |
|---|---|---|---|---|---|---|
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

By “domain linker” or grammatical equivalents herein is meant a linker that joins two protein domains together, such as those used in linking the different domains of a protein. Generally, there are a number of suitable linkers that can be used, including traditional peptide bonds, generated by recombinant techniques that allows for recombinant attachment of the two domains with sufficient length and flexibility to allow each domain to retain its biological function.

“Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, -233E or 233E designates an insertion of glutamic acid after position 233 and before position 234. Additionally, -233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233#, E233() or E233del designates a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it.

As used herein, “protein” herein is meant at least two covalently attached amino acids, which includes proteins, polypeptides, oligopeptides and peptides. The peptidyl group comprises naturally occurring amino acids and peptide bonds. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

As used herein, “domain” is meant protein domain, a part of a given protein sequence and tertiary structure that can function, and exist independently of the rest of the protein chain. Each domain forms a compact three-dimensional structure and often can be independently stable and folded. Domain varies in length, and is at least 10 amino acids long. Because they are independently stable, domains can be “swapped” by genetic engineering between one protein and another to make chimeric proteins.

By “residue” as used herein is meant a position in a protein and its associated amino acid identity.

By “Fab” or “Fab region” as used herein is meant the polypeptide that comprises the VH, CH1, VL, and CL immunoglobulin domains. Fab may refer to this region in isolation, or this region in the context of a full length antibody or antibody fragment.

By “Fv” or “Fv fragment” or “Fv region” as used herein is meant a polypeptide that comprises the VL and VH domains of a single antibody. As will be appreciated by those in the art, these are made up of two domains, a variable heavy domain and a variable light domain.

By “amino acid” and “amino acid identity” as used herein is meant one of the 20 naturally occurring amino acids that are coded for by DNA and RNA.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. In the present case, for example, the target antigen of interest herein can be a tumor associated antigen (TAA) including a CD19 protein. Thus, an “anti-CD19 binding domain” is an antigen binding domain (ABD) where the antigen is CD19. Additional targets are outlined below.

By “target cell” as used herein is meant a cell that expresses a target antigen.

By “variable domain” as used herein is meant the region of an immunoglobulin that comprises one or more Ig domains substantially encoded by any of the Vκ (V.kappa), Vλ (V.lamda), and/or VH genes that make up the kappa, lambda, and heavy chain immunoglobulin genetic loci respectively. Thus a “variable heavy domain” comprises (VH)FR1-vhCDR1-(VH)FR2-vhCDR2-(VH)FR3-vhCDR3-(VH)FR4 and a “variable light domain” comprises (VL)FR1-vlCDR1-(VL)FR2-vlCDR2-(VL)FR3-vlCDR3-(VL)FR4.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally recombinant. “Recombinant” means the antibodies are generated using recombinant nucleic acid techniques in exogenous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art. In some embodiments, the method for determining the KD of an antibody is by using surface plasmon resonance, for example, by using a biosensor system such as a BIACORE® system. In some embodiments, the KD of an antibody is determined by Bio-Layer Interferometry. In some embodiments, the KD is measured using flow cytometry with antigen-expressing cells. In some embodiments, the KD value is measured with the antigen immobilized. In other embodiments, the KD value is measured with the antibody (e.g., parent mouse antibody, chimeric antibody, or humanized antibody variants) immobilized. In certain embodiments, the KD value is measured in a bivalent binding mode. In other embodiments, the KD value is measured in a monovalent binding mode. Specific binding for a particular antigen or an epitope can be exhibited, for example, by an antibody having a KD for an antigen or epitope of at least about 10-7 M, at least about 10-8 M, at least about 10-9 M, at least about 10-10 M, at least about 10-11 M, at least about 10-12 M, at least about 10-13 M, or at least about 10-14 M. Typically, an antibody that specifically binds an antigen will have a KD that is 20-, 50-, 100-, 500-, 1000-, 5,000-, 10,000- or more times greater for a control molecule relative to the antigen or epitope.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M.O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of an algorithm to determine percent identity of a sequence is provided by the Genetics Computer Group (Madison, WI) in the “BestFit” utility application. The default parameters for this method are described in the Wisconsin Sequence Analysis Package Program Manual, Version 8 (1995) (available from Genetics Computer Group, Madison, WI). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code = standard; filter = none; strand = both; cutoff = 60; expect = 10; Matrix = BLOSUM62; Descriptions = 50 sequences; sort by = HIGH SCORE; Databases = non-redundant, GenBank + EMBL + DDBJ + PDB + GenBank CDS translations + Swiss protein + Spupdate + PIR. Details of these programs can be found at the internet address located by placing http:// in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

The term “nucleic acid” includes RNA or DNA molecules having more than one nucleotide in any form including single-stranded, double-stranded, oligonucleotide or polynucleotide. The term “nucleotide sequence” includes the ordering of nucleotides in an oligonucleotide or polynucleotide in a single-stranded form of nucleic acid.

A “vector” is capable of transferring gene sequences to a target cell. Typically, “vector construct,” “expression vector,” and “gene transfer vector,” mean any nucleic acid construct capable of directing the expression of a gene of interest and which can transfer a gene sequence to a target cell, which can be accomplished by genomic integration of all or a portion of the vector, or transient or inheritable maintenance of the vector as an extrachromosomal element. Thus, the term includes cloning, and expression vehicles, as well as integrating vectors.

The term “tumor-associated antigen” or “TAA” includes any antigenic substance produced on tumor cells. Tumor-associated antigen includes an antigen which is present only on tumor cells and not on non-tumor cell, and an antigen which is present on some tumor cells and also some normal cells.

As used herein, “single chain variable fragment” or “scFv” refers to an antibody fragment comprising a variable heavy domain and a variable light domain, wherein the variable heavy domain and a variable light domain are contiguously linked via a short flexible polypeptide linker, and capable of being expressed as a single polypeptide chain, and wherein the scFv retains the specificity of the intact antibody from which it is derived. The variable heavy domain and a variable light domain of a scFv can be, e.g., in any of the following orientations: variable light domain - scFv linker - variable heavy domain or variable heavy domain - scFv linker - variable light domain.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 comprises a phenylalanine at EU position 296, a F296Y substitution in IgG2 is considered an IgG subclass modification. Similarly, because IgG1 has a proline at position 241 and IgG4 has a serine there, an IgG4 molecule with a S241P is considered an IgG subclass modification. Note that subclass modifications are considered amino acid substitutions herein.

By “non-naturally occurring modification” as used herein with respect to an IgG domain is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “effector function” as used herein is meant a biochemical event that results from the interaction of an antibody Fc region with an Fc receptor or ligand. Effector functions include but are not limited to ADCC, ADCP, and CDC.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cy2 and Cy3), and optionally all or a portion of the hinge region between CH1 (Cy1) and CH2 (Cy2). Thus, in some cases, the Fc domain includes, from N- to C-terminus, CH2-CH3 or hinge-CH2-CH3. In some embodiments, the Fc domain is that from IgG1, IgG2, IgG3 or IgG4, with IgG1 hinge-CH2-CH3 finding particular use in many embodiments. Additionally, in certain embodiments, wherein the Fc domain is a human IgG1 Fc domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. In some embodiments, as is more fully described below, amino acid modifications are made to the Fc region, for example to alter binding to one or more FcyR or to the FcRn.

By “Fc gamma receptor”, “FcyR” or “FcgammaR” as used herein is meant any member of the family of proteins that bind the IgG antibody Fc region and is encoded by an FcyR gene. In human this family includes but is not limited to FcyRI (CD64), including isoforms FcyRIa, FcyRIb, and FcyRIc; FcyRII (CD32), including isoforms FcyRIIa (including allotypes H131 and R131), FcyRIIb (including FcyRIIb-1 and FcyRIIb-2), and FcyRIIc; and FcyRIII (CD16), including isoforms FcyRIIIa (including allotypes V158 and F158) and FcyRIIIb (including allotypes FcyRIIb-NA1 and FcyRIIb-NA2) (Jefferis et al., 2002, Immunol Lett 82:57-65, entirely incorporated by reference), as well as any undiscovered human FcyRs or FcyR isoforms or allotypes. In some cases, as outlined herein, binding to one or more of the FcyR receptors is reduced or ablated. For example, reducing binding to FcyRIIIa reduces ADCC, and in some cases, reducing binding to FcyRIIIa and FcyRIIb is desired.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. As discussed herein, binding to the FcRn receptor is desirable, and in some cases, Fc variants can be introduced to increase binding to the FcRn receptor.

“Fc variant” or “variant Fc” as used herein is meant a protein comprising an amino acid modification in an Fc domain. The modification can be an addition, deletion, or substitution. The Fc variants of the present invention are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the wildtype amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

By “fusion protein” as used herein is meant covalent joining of at least two proteins or protein domains. Fusion proteins may comprise artificial sequences, e.g. a domain linker, an Fc domain (e.g., a variant Fc domain), a CID or CInD domain as described herein. By “Fc fusion protein” herein is meant a protein comprising an Fc region, generally linked (optionally through a domain linker, as described herein) to one or more different protein domains. In some instances, two Fc fusion monomers can form a homodimeric Fc fusion protein or a heterodimeric Fc fusion protein. In some embodiments, one monomer of the heterodimeric Fc fusion protein includes an Fc domain alone (e.g., an “empty Fc domain”) and the other monomer is an Fc fusion protein, comprising a CID domain or a CInD domain, as outlined herein. In some embodiments, one monomer of a heterodimeric Fc fusion protein includes an Fc domain linked to a CID domain or a CInD domain, and the other monomer comprises an Fc domain linked to a therapeutic moiety. In some other embodiments, both the first and second monomers are Fc fusion proteins that include an Fc domain and a CInD domain.

By “fused” or “covalently linked” is herein meant that the components (e.g., a CID domain and an Fc domain) are linked by peptide bonds, either directly or indirectly via domain linkers, outlined herein.

By “heavy constant region” herein is meant the CH1-hinge-CH2-CH3 portion of a IgG antibody.

By “light constant region” is meant the CL domain from kappa or lambda.

### DETAILED DESCRIPTION OF THE INVENTION

As will be appreciated in the art, the compositions of the invention can take a variety of configurations, linking the components of the invention in various conformations. In general, as outlined herein, the compositions of the invention rely on one of two mechanisms: either the monomer components are held together with a small molecule for function, with the removal of the small molecule causing disassociation of the monomer components and subsequent clearance from the patient. These embodiments rely on CID small molecules and are generally depicted in FIG. 1. Alternatively, the compositions of the invention self associate in the absence of the small molecule but disassociate by the addition of the small molecule; these embodiments rely on CInD small molecules and are generally depicted in FIGS. 2, 3 and 4.

These systems are used to increase the half-life of the therapeutic moieties by using either an Fc domain or HSA, both of which are well known in the art to increase the serum half life of the molecules to which they are attached. By using the CID domains and a small molecule (CIDSM), the association of the Fc domain or the HSA domain with the therapeutic moiety is controlled: in the presence of the small molecule, the CID domains associate, thus incorporating the half-life extension moiety into the composition containing the therapeutic moiety. If the CIDSM is removed (or no longer administered to the patient), the association of the two “halves” is stopped, and the therapeutic moiety is rapidly cleared.

### A. Compositions Comprising CIDs

The invention provides compositions and methods for temporal control of the half-life of a T cell engager domain in the serum of a patient. As outlined herein, the compositions comprise a variety of different components, associated in particular ways, as described herein. In general, the compositions of the invention comprise a first and a second monomer, that are generally brought together non-covalently in the presence of a CID small molecule. Various embodiments of the composition are described herein.

Accordingly, one aspect of the invention involves a composition comprising a heterodimeric Fc fusion protein and a fusion protein moiety, as generally depicted in FIG. 1A. The heterodimeric Fc fusion protein is fused by a first and a second monomer. The first monomer comprises one half of a CID domain (herein referred to as “first CID domain”) covalently linked to a first Fc domain, optionally via a domain linker. In some embodiments, the first monomers comprise, from N- to C-terminal, the first CID domain-domain linker-Fc domain, and in additional embodiments the N- to C-terminal order is Fc domain-domain linker-the first CID domain. The second monomer comprises an empty Fc domain. The fusion protein moiety comprises a therapeutic moiety covalently linked to the other half of the CID, referred herein as “second CID domain”, optionally via a domain linker. In some embodiments, the fusion protein moiety comprises, from N- to C-terminal, the second CID domain-domain linker-therapeutic moiety, and in additional embodiments the N- to C-terminal order is therapeutic moiety-domain linker-the second CID domain. Addition of a CID small molecule induces association of the two halves of the CID, thereby enabling association of the therapeutic moiety with the Fc domain, and extending the serum half-life of the therapeutic moiety. In the event that the therapeutic moiety needs to be cleared from the blood quickly, the administration of the CID small molecule is ceased, leading to dissociation of the two halves of the CID and the dissociation of the therapeutic moiety from the Fc domain. Various embodiments of the composition are described herein.

### 1. First Monomers

As will be appreciated by those in the art, the compositions of the invention include several different fusion proteins with different functionalities.

In some embodiments the invention utilizes HSA as the half-life extension moiety and provides compositions comprising a first monomer comprising a first CID domain, a domain linker and an HSA binding domain, as generally depicted in FIG. 29. In some embodiments, the first monomers comprise, from N- to C-terminal, the first CID domain-domain linker-HSA binding domain, and in additional embodiments the N- to C-terminal order is HSA binding domain-domain linker-CID domain.

In some embodiments, the first monomers of the invention utilize Fc domains as the half-life extension moieties and comprise three components, a CID domain, a domain linker, and an Fc domain, in various configurations as outlined herein.

### a. CID Domains

Chemically induced dimerization is a biological mechanism in which two proteins non-covalently associate or bind only in the presence of a dimerizing agent. In the present invention, the dimerization agent is referred to as a “Chemically Induced Dimerizer small molecule” or a “CID small molecule” or “CIDSM”.

In the present invention, CID domains come in pairs that will associate in the presence of a CIDSM. As will be appreciated by those in the art, some CID pairs are identical, e.g., both of the CID domains are the same, and are brought together by the CIDSM. In other embodiments, the CID pairs are made up of two different CID domains that are brought together by the CIDSM.

In some embodiments of the present invention, a CID pair is derived from naturally occurring binding partners of a CIDSM. For example, a CID is composed of two FKBP halves, which dimerize in the presence of FK1012 (see, Fegan, A et al., Chemical Reviews. 110 (6): 3315-36); a CID is composed of two variant FKBP halves, which dimerize in the presence of rimiducid (see, Clackson T et al., Proc Natl Acad Sci USA. 95(18):10437-42); one half of the CID is FKBP, and the other half of the CID is Calcineurin, which dimerize in the presence of FK506 (Ho, SN et al., Nature. 382 (6594): 822-6); one half of the CID is FKBP, and the other half of the CID is CyP-Fas, which dimerize in the presence of FKCsA (Belshaw, PJ et al., Proc Natl Acad Sci USA. 93(10): 4604-7.); one half of the CID is FKBP, and the other half of the CID is FRB, which dimerize in the presence of Rapamycin (Rivera, VM et al., Nature Medicine. 2 (9): 1028-32.); one half of the CID is variant FKBP, and the other half of the CID is variant FRB, which dimerize in the presence of Rapamycin analogs ( J. Henri Bayle et al., Chemistry and Biology Vol 13, Issue 1, page 99-107); one half of the CID is GyrB, and the other half of the CID is GyrB, which dimerize in the presence of Courmermycin (Farrar, MA et al., Nature. 383 (6596): 178-81); one half of the CID is GAI, and the other half of the CID is GID1, which dimerize in the presence of Gibberellin ( Miyamoto, T et al., Nature Chemical Biology. 8 (5): 465-70); one half of the CID is SNAP-tag, and the other half of the CID is HaloTag, which dimerize in the presence of HaXS (Erhart, D et al., Chemistry and Biology. 20 (4): 549-57); and one half of the CID is eDHFR, and the other half of the CID is HaloTag, which dimerize in the presence of TMP-tag (Ballister, E et al., Nature Communications. 5 (5475)).

In some embodiments of the present invention, the first CID domain is a naturally occurring binding partner of the CID small molecule, and the second CID domain is an antigen binding domain (ABD) that binds specifically to the complex formed between the first CID domain and the CIDSM, but does not bind to the first CID domain without the CID small molecule and does not bind to the free small molecule. Examples can be found in WO2018/213848, which is incorporated herein by reference. This second CID domain in this context can also be referred to as a “CID-ABD”; that is, an antigen binding domain that binds to the first CID domain and the CIDSM.

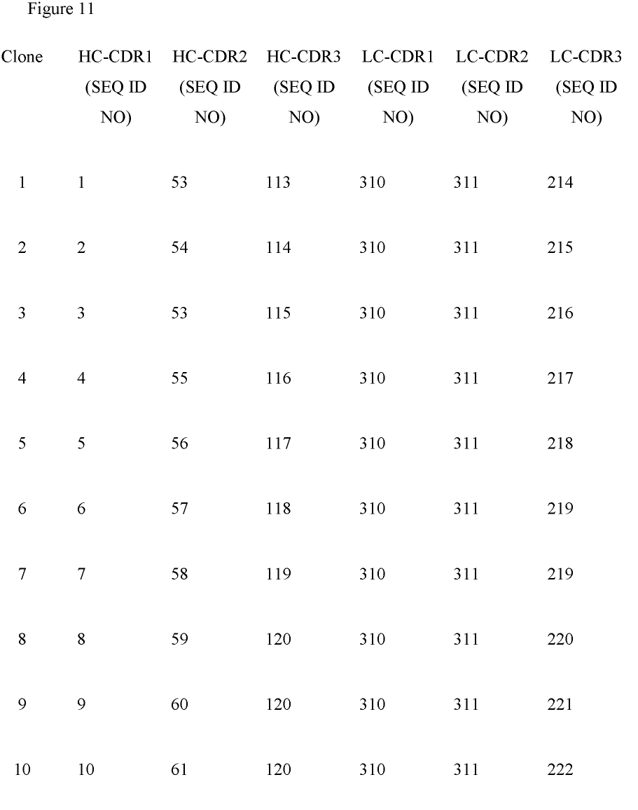

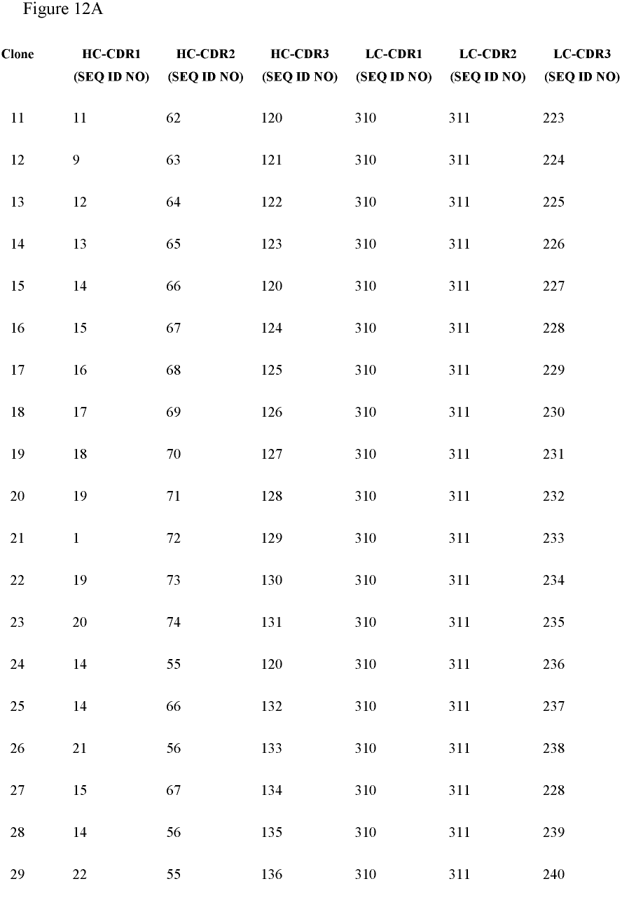

For example, in some embodiments, the first CID domain is an ABT-737 binding domain of Bcl-xL and the CID small molecule is ABT-737. The second CID domain comprises a heavy chain variable domain and light chain variable domain comprising the amino acid sequences of vhCDRs and vlCDRs as shown in FIG. 11. The second CID domain binds specifically to the complex formed between the first CID domain and the CID small molecule, but does not bind to the first CID domain without the CID small molecule and does not bind to the free CID small molecule. In some embodiments, the ABT-737 binding domain of Bcl-xL comprises the amino acid sequence of SEQ ID NO: 314.

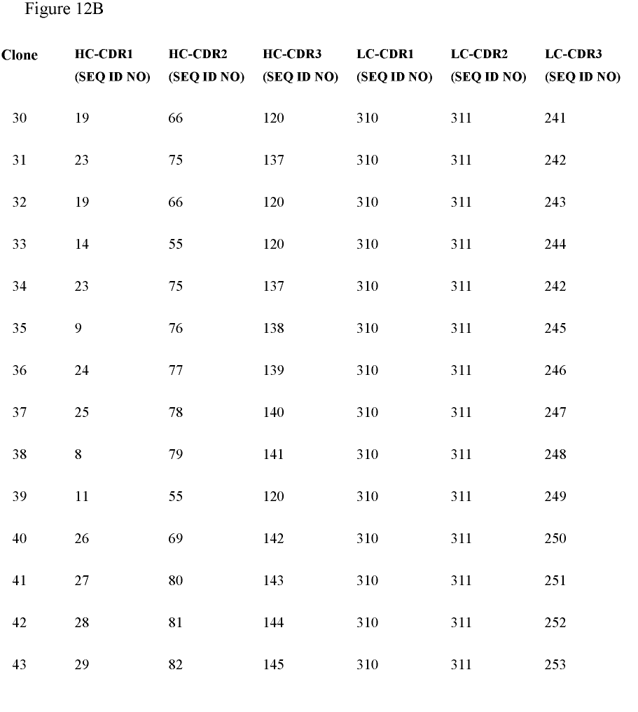

In an additional embodiment, the first CID domain is an ABT-199 binding domain of BCl-2 or BCl-2 (C158A) and the CID small molecule is ABT-199 (venetoclax). The second CID domain comprises a heavy chain variable domain and light chain variable domain comprising the amino acid sequences of vhCDRs and vlCDRs as shown in FIGS. 12A-12B. The second CID domain binds specifically to the complex formed between the first CID domain and the CID small molecule, but does not bind to the first CID domain without the small molecule and does not bind to the free small molecule. In some embodiments, the ABT-199 binding domain of BCl-2 or BCl-2 (C158A) comprises the amino acid sequence of SEQ ID NO: 315.

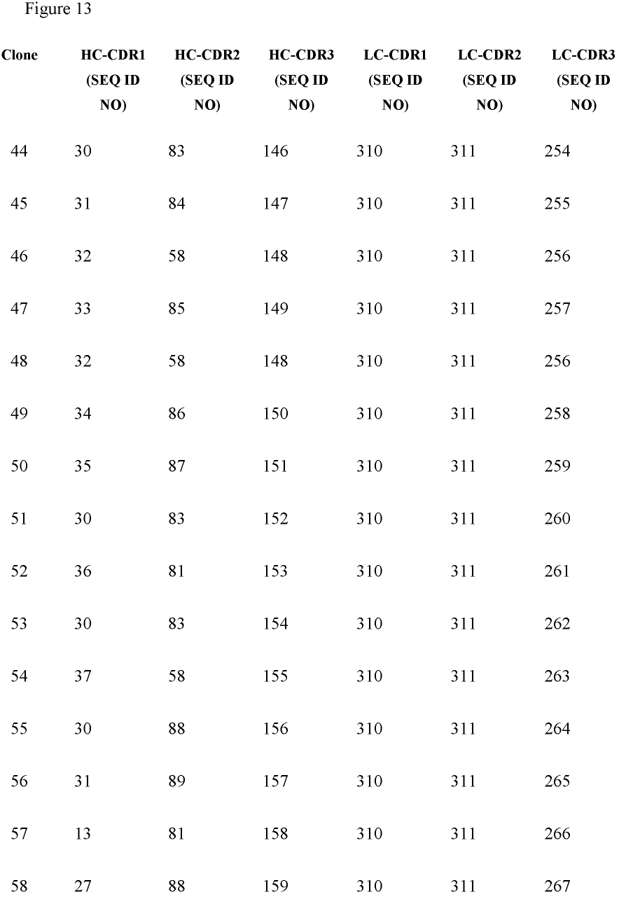

In some embodiments, the first CID domain is an ABT-263 binding domain of BCL-2 and the CID small molecule is ABT-263. The second CID domain comprises a heavy chain variable domain and light chain variable domain comprising the amino acid sequences of vhCDRs and vlCDRs as shown in FIG. 13. The second CID domain binds specifically to the complex formed between the first CID domain and the CID small molecule, but does not bind to the first CID domain without the CID small molecule and does not bind to the free CID small molecule. In some embodiments, the ABT-263 binding domain of BCl-2 comprises the amino acid sequence of SEQ ID NO: 315.

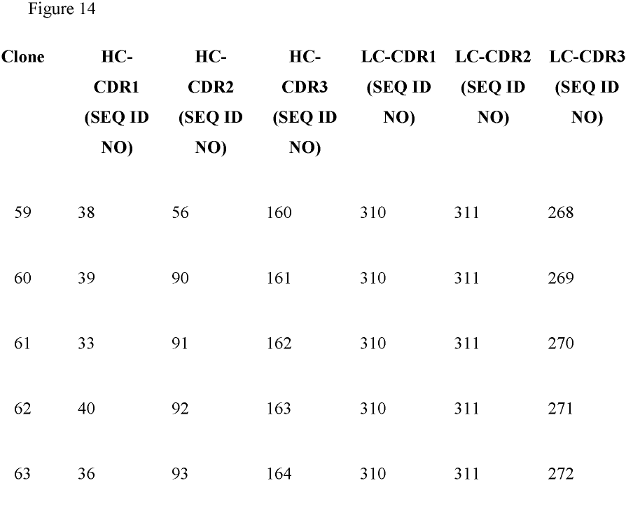

In additional embodiments, the first CID domain is a LCL161 binding domain of cIAPl and the CID small molecule is LCL161. The second CID domain comprises a heavy chain variable domain and light chain variable domain comprising the amino acid sequences of vhCDRs and vlCDRs as shown in FIG. 14. The second CID domain binds specifically to the complex formed between the first CID domain and the CID small molecule, but does not bind to the first CID domain without the CID small molecule and does not bind to the free CID small molecule. In some embodiments, the LCL161 binding domain of cIAPl comprises the amino acid sequence of SEQ ID NO: 317.

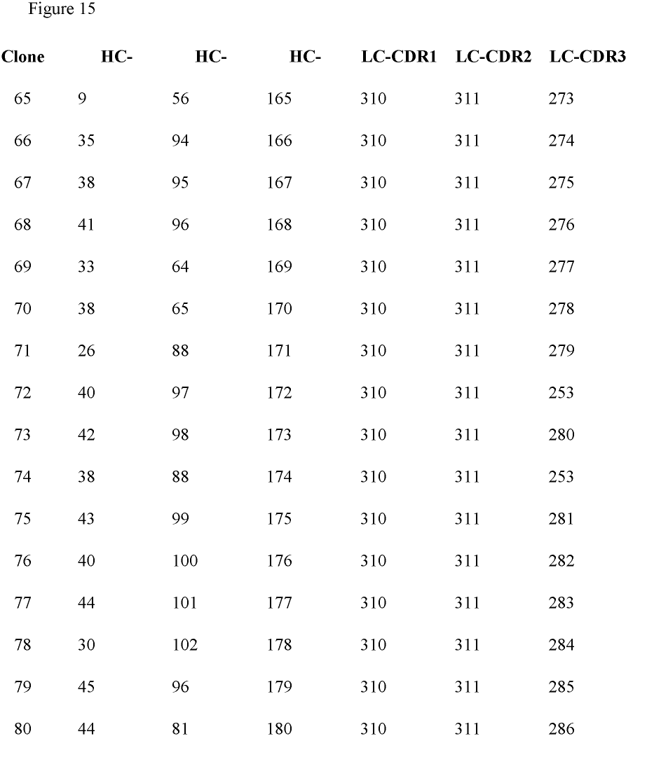

In additional embodiments, the first CID domain is a GDC-0152 binding domain of cIAPl and the CID small molecule is GDC-0152. The second CID domain comprises a heavy chain variable domain and light chain variable domain comprising the amino acid sequences of vhCDRs and vlCDRs as shown in FIG. 15. The second CID domain binds specifically to the complex formed between the first CID domain and the CID small molecule, but does not bind to the first CID domain without the CID small molecule and does not bind to the free CID small molecule. In some embodiments, the GDC-0152 binding domain of cIAPl comprises the amino acid sequence of SEQ ID NO: 317.

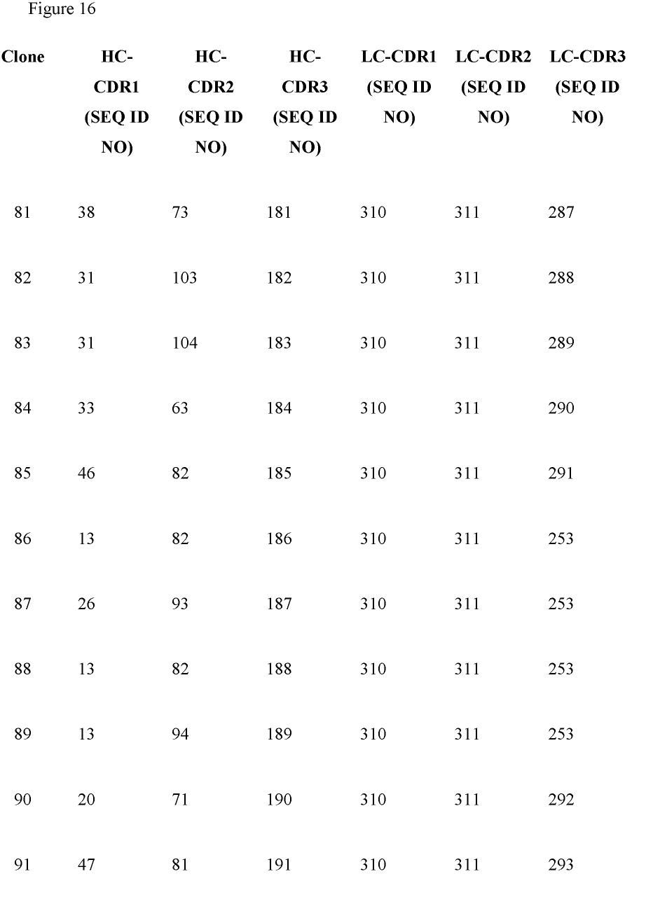

In additional embodiments, the first CID domain is a AT406 binding domain of cIAPl and the CID small molecule is AT406. The second CID domain comprises a heavy chain variable domain and light chain variable domain comprising the amino acid sequences of vhCDRs and vlCDRs as shown in FIG. 16. The second CID domain binds specifically to the complex formed between the first CID domain and the CID small molecule, but does not bind to the first CID domain without the CID small molecule and does not bind to the free CID small molecule. In some embodiments, the AT406 binding domain of cIAPl comprises the amino acid sequence of SEQ ID NO: 317.

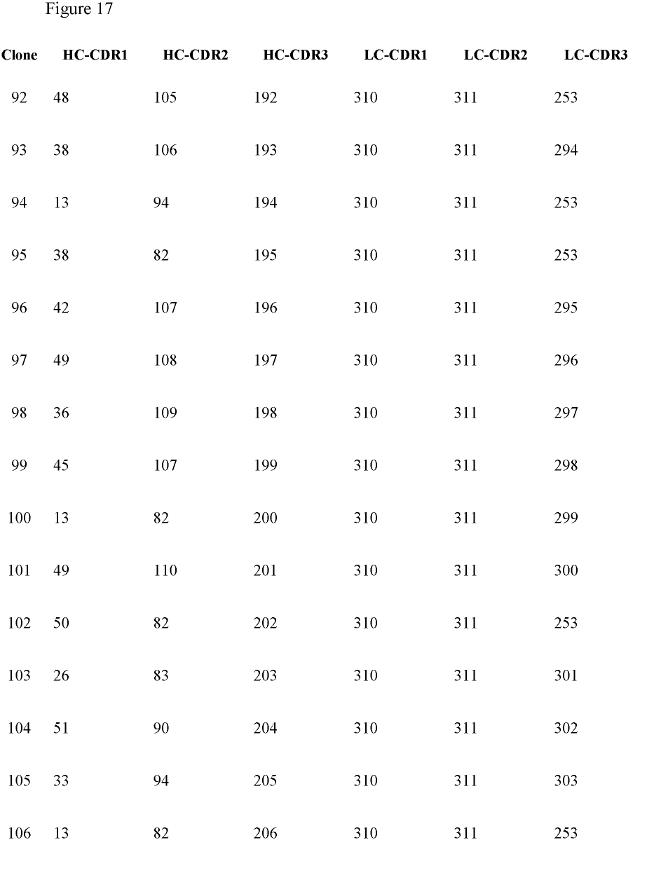

In additional embodiments, the first CID domain is a CUDC-427 binding domain of cIAPl and the CID small molecule is CUDC-427. The second CID domain comprises a heavy chain variable domain and light chain variable domain comprising the amino acid sequences of vhCDRs and vlCDRs as shown in FIG. 17. The second CID domain binds specifically to the complex formed between the first CID domain and the CID small molecule, but does not bind to the first CID domain without the CID small molecule and does not bind to the free CID small molecule. In some embodiments, the CUDC-427 binding domain of cIAPl comprises the amino acid sequence of SEQ ID NO: 317.

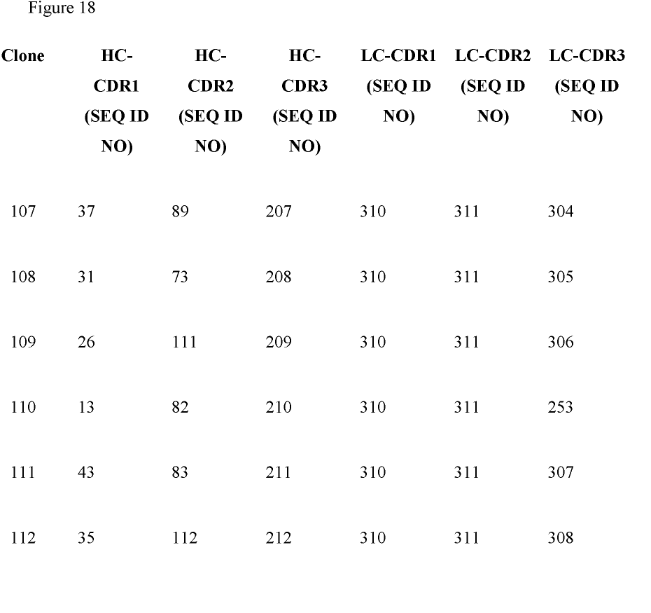

In additional embodiments, the first CID domain is a synthetic ligand of rapamycin (SLF) binding domain of FKBP and the CID small molecule is SLF. The second CID domain comprises a heavy chain variable domain and light chain variable domain comprising the amino acid sequences of vhCDRs and vlCDRs as shown in FIG. 18. The second CID domain binds specifically to the complex formed between the first CID domain and the CID small molecule, but does not bind to the first CID domain without the CID small molecule and does not bind to the free CID small molecule. In some embodiments, the SLF binding domain of FKBP comprises the amino acid sequence of SEQ ID NO: 316.

In some other embodiments of the present invention, both CID domains are antigen binding domains (ABDs). The first CID domain binds specifically to the CID small molecule which acts as the antigen, and the second CID domain binds specifically to the complex formed between the first CID domain and the CID small molecule, but does not bind to the first CID domain or the free CID small molecule.

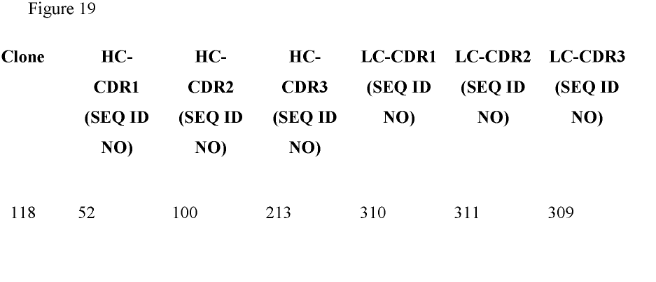

In some embodiments, the CID small molecule is methotrexate, and the first CID domain is a methotrexate ABD which comprises a heavy chain variable domain and light chain variable domain comprising the amino acid sequences of vh-CDR1, vh-CDR2, vh-CDR3, vl-CDR1, vl-CDR2, and vl-CDR3 as SEQ ID NOs: 319, 320, 321, 322, 323 and 324, respectively. The second CID domain comprises an ABD capable of specifically binding to the complex between methotrexate and the first CID domain, and the second CID domain comprises vhCDRs and vlCDRs as shown in FIG. 19. In some embodiments, the methotrexate ABD is a methotrexate-binding Fab as described in Gayda et al. Biochemistry 2014 53 (23), 3719-3726.

In some embodiments, the second half of the CID comprises an ABD and binds to a site of the complex comprising at least a portion of the small molecule and a portion of the first half of the CID. In some embodiments, the second half of the CID comprises an ABD, and binds to a site of the complex of the small molecule and the first half of the CID, wherein the second half of the CID binds to the site comprising at least one atom of the small molecule and one atom of the first half of the CID.

In some embodiments, the second half of the CID binds to the complex of the first half of the CID and the small molecule with a dissociation constant (KD) no more than about 1/250 times (such as no more than about any of 1/300, 1/350, 1/400, 1/450, 1/500, 1/600, 1/700, 1/800, 1/900, 1/1000, 1/1 100, 1/1200, 1/1300, 1/1400, or 1/1500 times, or less) its KD for binding to each of the free small molecule and the free first half of the CID.

Binding moieties that specifically bind to a complex between a small molecule and a cognate binding moiety can be produced according to methods known in the art, see, for example, WO2018/213848, hereby incorporated herein by reference in its entirety and specifically for the methods for producing CID domains. Briefly, a screening is performed from an antibody library, a DARPin library, a nanobody library, or an aptamer library or a phage displayed Fab library. For example, as the step 1, binding moieties can be selected that do not bind to the cognate binding moiety in the absence of the small molecule, thereby generating a set of counter selected binding moieties; and then, as step 2, the counter selected binding moieties can be screened for binding moieties that bind to the complex of the small molecule and the cognate binding moiety, thereby generating a set of positively selected binding moieties. Steps 1 and 2 of screening can be conducted one or more rounds, wherein each round of screening comprises the screening of step 1 and the screening of step 2, such that a set of binding moieties that specifically bind to the complex between the small molecule and the cognate binding moiety is generated. In some embodiments, two or more rounds of screening are performed, wherein the input set of binding moieties of step 1 for the first round of screening is the binding molecule library; the input set of binding moieties of step 2 for each round of screening is the set of counter selected binding moieties of step 1 from the given round of screening; the input set of binding moieties of step 1 for each round of screening following the first round of screening is the set of positively selected binding moieties of step 2 from the previous round of screening; and the set of binding moieties that specifically bind to the complex between the small molecule and the cognate binding moiety is the set of positively selected binding moieties of step 2 for the last round of screening.

Phage display screening can be done according to previously established protocols (see, Seiler, et al, Nucleic Acids Res., 42:D12531260 (2014). For example, to select antibody binding moieties for the complex of BCL-xL and ABT-737, antibody phage library can be screen against biotinylated BCL-xL captured with streptavidincoated magnetic beads (Promega). Prior to each selection, the phage pool can be incubated with 1 mM of BCL-xL immobilized on streptavidin beads in the absence of ABT-737 in order to deplete the library of any binders to the apo form of BCL-xL. Subsequently, the beads can be removed and ABT-737 can be added to the phage pool at a concentration of 1 mM. In total, four rounds of selection can be performed with decreasing amounts of BCL-xL antigen (100 nM, 50 nM, 10 nM and 10 nM). To reduce the deleterious effects of nonspecific binding phage, specific BCL-xL binding Fab-phage can be selectively eluted from the magnetic beads by addition of 2 g/mL TEV protease. Individual phage clones from the fourth round of selection can then be analyzed for sequencing.

### b. HSA Binding Domains

In addition to the first CID domains, some embodiments rely on first monomers of the invention that also include an HSA binding domain as the half-life extension moiety. In some embodiments, the HSA binding domain comprises an antigen binding domain derived from a monoclonal antibody, a polyclonal antibody, a recombinant antibody, a human antibody, or a humanized antibody. The anti-HSA antigen binding domain can take any format, including but not limited to a full antibody, an Fab, an Fv, a single chain variable fragments (scFv), a single domain antibody such as a heavy chain variable domain (VH), a light chain variable domain (VL) and a variable domain (VHH) of camelid derived single domain antibody.

The binding affinity of the HSA binding domain can be selected so as to target a specific serum half-life of a T cell engager. Thus, in some embodiments, the HSA binding domain has a high binding affinity to HSA. In other embodiments, the HSA binding domain has a medium binding affinity to HSA. In yet other embodiments, the HSA binding domain has a low or marginal binding affinity to HSA. Exemplary binding affinities include KD concentrations at 10 nM or less (high), between 10 nM and 100 nM (medium), and greater than 100 nM (low). As above, binding affinities to HSA are determined by known methods such as Surface Plasmon Resonance (SPR).

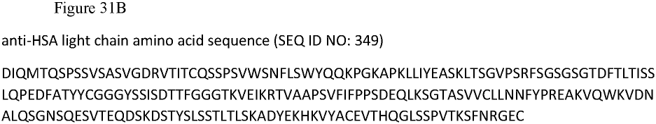

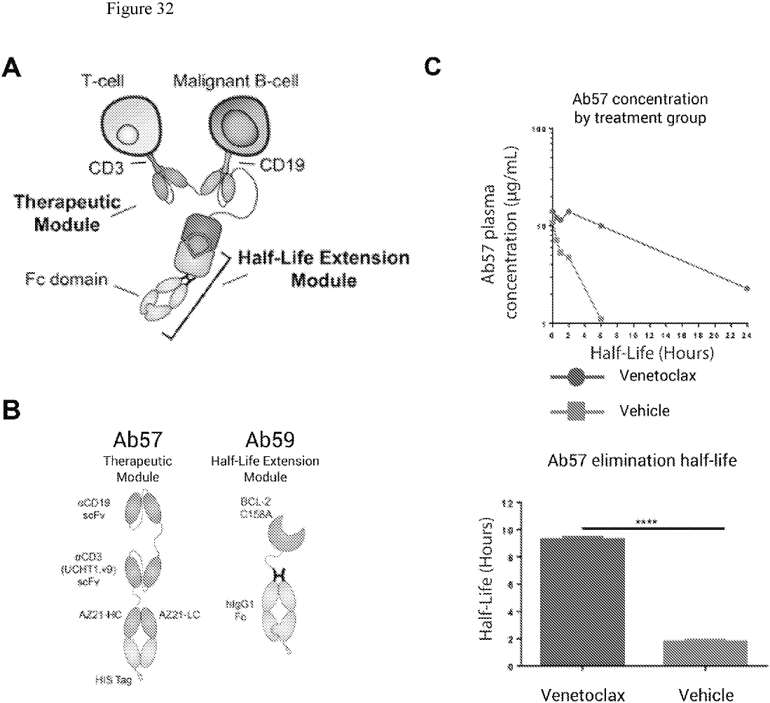

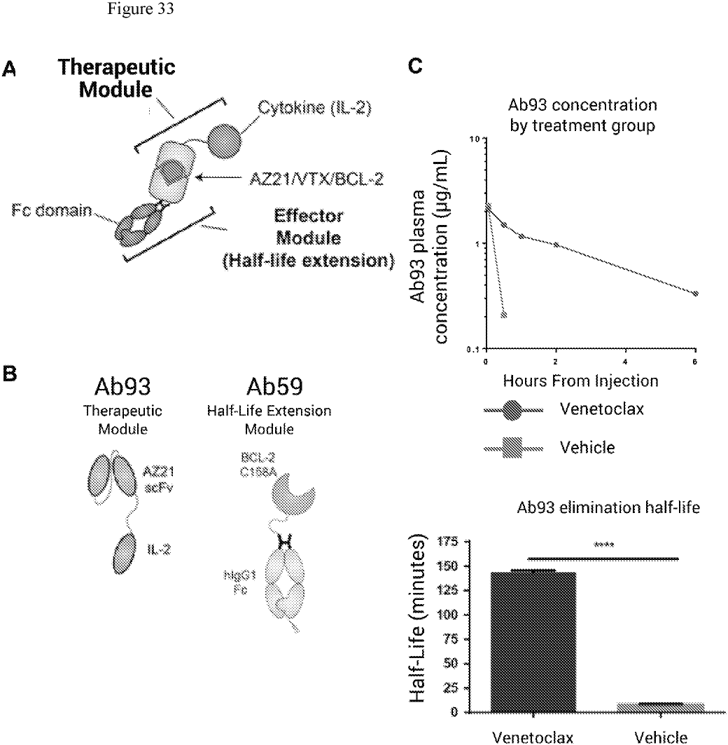

In some embodiments, the HSA binding domain is an antigen binding domain comprising an scFv that binds to HSA. In some embodiments, the HSA binding domain is an sdABD. In some embodiments, the HSA binding domain is the HSA binding domain of Streptococcal protein G. In some embodiments, the HSA binding domain is a humanized anti-HSA binding fragment, such as a humanized scFv or sdABD. In some embodiments, the HSA binding domain comprises a heavy chain variable domain of SEQ ID NO:339 and a light chain variable domain of SEQ ID NO:340. In some embodiments, the HSA binding domain is an sdABD and comprises a single monomeric variable domain selected from SEQ ID NO: 341, 342, 343, 344, 345, 346 and 347. In some embodiments, the HSA binding domain is modified to increase or decrease its affinity to HSA, for example using methods shown in Ralph et al., MABS. 2016, VOL. 8, NO. 7, 1336-1346. In some embodiments, the HSA binding domain comprises a heavy chain of SEQ ID NO:348 and a light chain of SEQ ID NO:349. Exemplary amino acid sequences are shown in FIG. 31A and FIG. 31B.

### c. Domain Linker

In many embodiments herein, domain linkers are used to link the various components of the invention together such that the biological function of the component is retained.

A domain linker can serve, for example, simply as a convenient way to link the two entities, as a means to spatially separate the two entities. A domain linker may have a length that is adequate to link two molecules in such a way that they assume the correct conformation relative to one another so that they retain the desired activity. In general, a linker joining two domains can be designed to (1) allow the two domains to fold and act independently of each other, (2) not have a propensity for developing an ordered secondary structure which could interfere with the functional domains of the two domains, (3) have minimal hydrophobic or charged characteristic which could interact with the functional protein domains and/or (4) provide steric separation of the two domains. A domain linker can also be used to provide, for example, lability to the connection between two domains, an enzyme cleavage site (for example a cleavage site for a protease), a stability sequence, a molecular tag, a detectable label, or various combinations thereof.

The length and composition of a domain linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, a domain linker may be a peptide which includes the following amino acid residues: Gly, Ser, Ala, or Thr. In some embodiments, the linker peptide is from about 1 to 50 amino acids in length, about 1 to 30 amino acids in length, about 1 to 20 amino acids in length, or about 5 to about 10 amino acids in length. Exemplary peptide linkers include glycine-serine polymers such as (GS)n, (GGS)n, (GGGS)n, (GGSG)n (GGSGG)n, (GSGGS)n, and (GGGGS)n, wherein n is an integer of at least one (e.g.,1, 2, 3, 4, 5, 6, 7, 8, 9, or 10); glycine-alanine polymers; alanine-serine polymers; and other flexible linkers.

Alternatively, a variety of non-proteinaceous polymers can be used as a domain linker, including but not limited to polyethylene glycol (PEG), polypropylene glycol, polyoxyalkylenes, or copolymers of polyethylene glycol and polypropylene glycol.

A domain linker may also be derived from immunoglobulin light chain, for example [symbol] or [symbol] . Linkers can also be derived from immunoglobulin heavy chains of any isotype, including for example [symbol] , and [symbol] . For example, domain linkers can include any sequence of any length of CL/CH1 domain but not all residues of CL/CH1 domain; for example, the first 5-12 amino acid residues of the CL/CH1 domains.

A domain linker may also be derived from other proteins such as Ig-like proteins (e.g., TCR, FcR, KIR), hinge region-derived sequences, and other natural sequences from other proteins.

In some embodiments of the invention, a first CID domain is linked to a Fc domain via a first domain linker. In some embodiments, a second CID domain linked to a therapeutic moiety via a second domain linker. The first and the second domain linker may or may not be the same.

In some embodiments of the invention, a first CID domain is linked to a HSA binding domain via a first domain linker. In some embodiments, a second CID domain linked to a therapeutic moiety via a second domain linker. The first and the second domain linker may or may not be the same.

In some embodiments, the domain linker serves to link the VH and VL domains of an Fv together to form a scFv, and can be referred to as a “scFv linker”. In these embodiments, the scFv linker is long enough to allow the VH and VL domains to properly associate. In some embodiments, the scFv linker is from 10 to 25 amino acids in length.

### d. Fc Domains

In some embodiments, particularly those utilizing Fc domains as the half life extension moieties, the invention provides heterodimeric Fc fusion proteins that include a first monomer that includes a first Fc domain and a first CID domain, and a second monomer that includes a second Fc domain (e.g., an “empty Fc domain”). The Fc fusion proteins are based on the self-assembling nature of the two Fc domains on each monomer. Heterodimeric Fc domains are made by altering the amino acid sequence of the Fc domain in each monomer to “skew” the formation of heterodimeric Fc domains as more fully discussed below.

The Fc domains can be derived from IgG Fc domains, e.g., IgG1, IgG2, IgG3 or IgG4 Fc domains, with IgG1 Fc domains finding particular use in the invention. As described herein, IgG1 Fc domains may be used, often, but not always in conjunction with ablation variants to ablate effector function. Similarly, when low effector function is desired, IgG4 Fc domains may be used.

For any of the dimeric Fc fusion proteins described herein, the carboxyterminal portion of each chain defines a constant region primarily responsible for effector function. Kabat et al. collected numerous primary sequences of the variable regions of heavy chains and light chains. Based on the degree of conservation of the sequences, they classified individual primary sequences into the CDRs and the framework and made a list thereof (see SEQUENCES OF IMMUNOLOGICAL INTEREST, 5th edition, NIH publication, No. 91-3242, E.A. Kabat et al., entirely incorporated by reference). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

In some embodiments of the dimeric Fc fusion proteins described herein, each of the first and second monomers include an Fc domain that has the formula hinge-CH2-CH3, wherein the hinge is either a full or partial hinge sequence. In some embodiments of the dimeric Fc fusion proteins described herein, each of the first and second monomers include an Fc domain that has the formula CH2-CH3.

### (i) Heterodimeric Fc Variants

In embodiments utilizing Fc domains as the half-life extension moieties, the Fc fusion protein is a heterodimeric Fc fusion protein. Such heterodimeric proteins include two different Fc domains (one on each of the first and second monomers) that include modifications that facilitate the heterodimerization of the first and second monomers and/or allow for ease of purification of heterodimers over homodimers, collectively referred to herein as “heterodimerization variants.” As will be appreciated by those in the art, generally these heterodimeric monomers are made by including genes for each monomer into the host cells. This generally results in the formation of the desired heterodimer (A-B), as well as the two homodimers (A-A and B-B). As is known in the art, there are a number of mechanisms that can be used to generate the Fc heterodimers of the present invention. Thus, amino acid variants that lead to the production of heterodimers are referred to as “heterodimerization variants”. As discussed below, heterodimerization variants can include steric variants (e.g. the “knobs and holes” variants described below and the “charge pairs” variants described below) that “skew” the formation of A-B heterodimers over A-A and B-B homodimers.

One mechanism is generally referred to in the art as “knobs and holes”, or KIH referring to amino acid engineering that creates steric influences to favor heterodimeric formation and disfavor homodimeric formation. That is, one monomer is engineered to have a bulky amino acid (a “knob”) and the other is engineered to reduce the size of the amino acid side chain (a “hole”), that skews the formation of heterodimers over homodimers. These techniques and sequences are described in Ridgway et al., Protein Engineering 9(7):617 (1996); Atwell et al., J. Mol. Biol. 1997 270:26; U.S. Pat. No. 8,216,805, US 2012/0149876, all of which are hereby incorporated by reference in their entirety. The Figures of these references (also specifically incorporated by reference herein for the amino acid variants) identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” or “charge pairs” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are "monomer corresponding sets) and C220E/P228E/368E paired with C220R/E224R/P228R/K409R.

Heterodimerization variants can include skew variants (e.g., the “knobs and holes” and “charge pairs” variants described below). Exemplary methods include symmetric-to-asymmetric steric complementarity design, e.g., introducing KiH, HA-TF, and ZW1 mutations [see, Atwell et al., J Mol Biol (1997) 270(1):26-35; Moore et al., MAbs (2011) 3(6):546-57; Von Kreudenstein et al., MAbs (2013) 5(5):646-54, all of which are expressly incorporated herein by reference in their entirety]; charge-to-charge swap (e.g., introducing DD-KK mutations)(see, Gunasekaran et al., J Biol Chem 2010; 285:19637-46 incorporated herein by reference in its entirety); charge-to-steric complementarity swap plus additional long-range electrostatic interactions (e.g., introducing EW-RVT mutations) (Choi et al., Mol Cancer Ther (2013) 12(12):2748-59 incorporated herein by reference in its entirety); and isotype strand swap, e.g., introducing strand-exchange engineered domain (SEED) (Klein et al, MAbs (2012) 4(6):653-63; Von Kreudenstein et al., MAbs (2013) 5(5):646-54, all of which are expressly incorporated herein by reference in their entirety), as summarized in Table 2.

Table 2

| | | |
|---|---|---|
| Heterodimeric Fc domain name | Paired mutation - one Fc domain | Paired mutation - cognate Fc domain |
| KiH | T366W | T366S/L368A/Y407V |
| KiHS-S | T366W/S354C | T366S/L368A/Y407V/Y349C |
| HA-TF | S364H/F405A | Y349T/T394F |
| ZW1 | T350V/L351Y/F405A/ Y407V | T350V/T366L/K392L/T394W |
| 7.8.60 | K360D/D399M/Y407A | E345R/Q347R/T366V/K409V |
| DD-KK | K409D/K392D | D399K/E356K |
| EW-RVT | K360E/K409W | Q347R/D399V/F405T |
| EW-RVTS-S | K360E/K409W/Y349C | Q347R/D399V/F405T/S354C |
| SEED | IgA-derived 45 residues on IgG1 CH3 | IgG1-derived 57 residues on IgA CH3 |
| A107 | K370E/K409W | E357N/D399V/F405T |

In addition to heterodimerization variants, the dimeric Fc fusion proteins provided herein (both homodimeric and heterodimeric) may independently include Fc modifications that affect functionality including, but not limited to, altering binding to one or more Fc receptors (e.g., FcyR and FcRn).

### (ii) FcyR Variants

In some embodiments, the Fc fusion proteins includes one or more amino acid modifications that affect binding to one or more Fcγ receptors (e.g., “FcyR variants”). FcyR variants (e.g., amino acid substitutions) that result in increased binding as well as decreased binding can be useful. For example, it is known that increased binding to FcyRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcyRIIb (an inhibitory receptor) can be beneficial as well in some circumstances. FcyR variants that reduce FcyR activation and Fc-mediated toxicity such as P329G, L234A, L235A can find use in the Fc fusion proteins in the current invention (see, Schlothauer et al. Protein Eng Des Sel. 2016;29(10):457-466 incorporated herein for reference in its entirety). For example, IgG1 Fc domain incorporating P329G, L234A, L235A can be used in the current invention, and can be further modified to facilitate heterdimerization. Exemplary amino acid sequences are shown in FIG. Figue 28.

Additional FcyR variants can include those listed in U.S. Pat. Nos. 8,188,321 (particularly FIG. 41) and 8,084,582, and U.S. Publ. App. Nos. 20060235208 and 20070148170, all of which are expressly incorporated herein by reference in their entirety and specifically for the variants disclosed therein that affect Fcy receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

### (iii) FcRn Variants

Further, Fc fusion proteins described herein can independently include Fc substitutions that confer increased binding to the FcRn and increased serum half-life. Such modifications are disclosed, for example, in U.S. Pat. No. 8,367,805, hereby incorporated by reference in its entirety, and specifically for Fc substitutions that increase binding to FcRn and increase half-life. Such modifications include, but are not limited to 434S, 434A, 428L, 308F, 259I, 428L/434S, 259I/308F, 436I/428L, 436I or V/434S, 436V/428L and 259I/308F/428L.

### (iv) Ablation Variants

In some embodiments, the Fc fusion proteins described herein include one or more modifications that reduce or remove the normal binding of the Fc domain to one or more or all of the Fcγ receptors (e.g., FcγR1, FcyRIIa, FcyRIIb, FcyRIIIa, etc.) to avoid additional mechanisms of action. Such modifications are referred to as “FcyR ablation variants” or “Fc knock out (FcKO or KO)” variants. In some embodiments, particularly in the use of immunomodulatory proteins, it is desirable to ablate FcyRIIIa binding to eliminate or significantly reduce ADCC activity such that one of the Fc domains comprises one or more Fcy receptor ablation variants. These ablation variants are depicted in FIG. 31 of U.S. Pat. No. 10,259,887, which is herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. It should be noted that the ablation variants referenced herein ablate FcyR binding but generally not FcRn binding.

### 2. Second Monomers

In some embodiments, provided herein are heterodimeric Fc fusion proteins that include a first monomer that includes a first Fc domain and a first CID domain, and a second monomer that includes a second Fc domain. In some cases, the second monomer comprises just the Fc domain (e.g. an “empty Fc domain,”). Heterodimerization variants and other Fc variants for the second monomers are described herein.

In some other embodiments, as generally depicted in FIG. 29, present invention provides first and second monomers that associate in the presence of a CIDSM to result in the association of a half-life extension domain to a T cell engager domain, thus allowing temporal control of the half-life of the T cell engager domain. Thus, in addition to the first monomers discussed above, the invention provides compositions comprising a second monomer comprising a second CID domain, a domain linker and a T cell engager domain, as generally depicted in FIG. 29. In some embodiments, the second monomers comprise, from N- to C-terminal, the second CID domain-domain linker-T cell engager domain, and in additional embodiments the N- to C-terminal order is T cell engager domain-domain linker-CID domain.

The second CID domain and domain linker are as outlined herein.

### a. T Cell Engager Domains

In many embodiments, the therapeutic moiety is a T cell engager domain. In general, as is known in the art, a T cell engager domain comprises, at a minimum, an ABD that binds to a T Cell, generally to the CD3 protein expressed on the surface of the T cell, linked to an ABD that binds to a tumor associated antigen (TAA) on a cancer cell. These T cell engager domains are designed to allow specific targeting of cells expressing the target antigen by recruiting cytotoxic T cells. Accordingly, a T cell engager domain described herein can engage cytotoxic T cells via binding to the surface-expressed CD3 proteins, which form part of the T cell receptor (TCR). Simultaneous binding of a T cell engager to CD3 and to a target antigen expressed on the surface of particular tumor cells causes T cell activation and mediates the subsequent lysis of the particular target antigen-expressing cell. Thus, a T cell engager domain can induce strong, specific and efficient target cell killing (Ellerman, Methods, 2019; 54:102-107).

In some embodiments, the C terminus of a T cell ABD is linked to the N terminus of a TAA-ABD, via a domain linker. In other embodiments, the N terminus of a T cell binding-domain is linked to the C terminus of a target antigen-binding domain of a T cell engager via a domain linker.

### (i) T Cell ABD

The binding specificity of a T cell engager domain to T cells is mediated by the recognition of the TCR. As part of the TCR, CD3 is a protein complex that includes a CD3λ (gamma) chain, a CD3b (delta) chain, and two CD3ε (epsilon) chains which are present on the cell surface. CD3 associates with the α (alpha) and β (beta) chains of the TCR as well as CD3 (zeta) altogether to form the complete TCR. Clustering of CD3 on T cells, such as by immobilized anti-CD3 antibodies, leads to T cell activation similar to the engagement of the T cell receptor but independent of its clone typical specificity.

The T cell engager domain described herein comprises a domain which specifically binds to the TCR. In some embodiments, the T cell engager domain described herein comprises a domain which specifically binds to human CD3.

In some embodiments, the T cell ABD of the T cell engager domain comprises an antigen binding-domain derived from a monoclonal antibody, a polyclonal antibody, a recombinant antibody, a human antibody, or a humanized antibody. The T cell ABD can take any format, including but not limited to an Fv, a single chain variable fragments (scFv), a single domain antibody such as a heavy chain variable domain (VH), a light chain variable domain (VL) and a variable domain (VHH) of camelid derived single domain antibody.

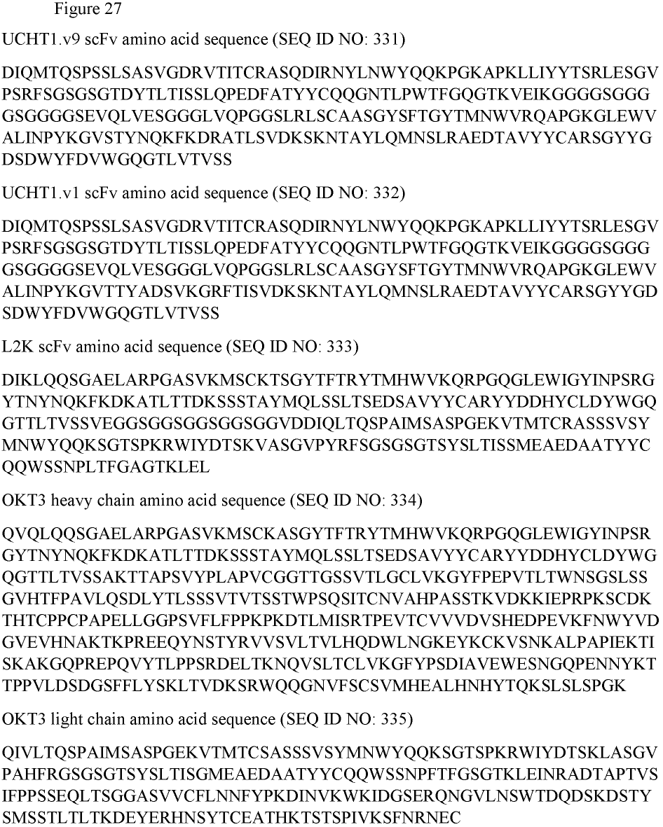

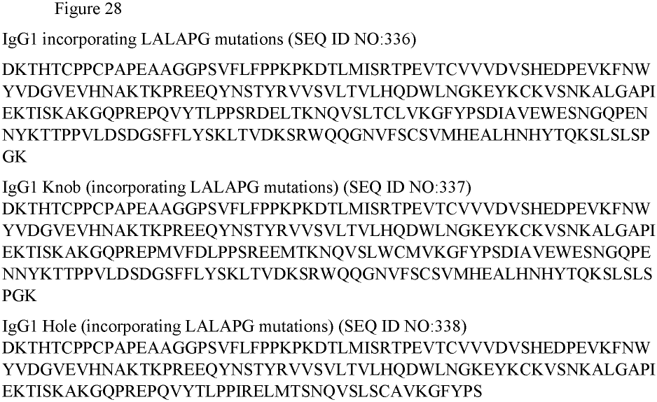

In some embodiments, the T cell ABD of the T cell engager moiety is an anti-CD3 ABD, which comprises a set of three light chain CDRs (v1CDR1, vlCDR2 and vlCDR3), and three heavy chain CDRs (vhCDR1, vhCDR2 and vhCDR3) of an anti-CD3 antibody. Exemplary anti-CD3 antibodies that contribute to the CDR sets, include, but are not limited to, L2K, UCHT1, variants of UCHT1 including UCHT1.v9, muromonab-CD3 (OKT3), otelixizumab (TRX4), teplizumab (MGA031), visilizumab (Nuvion), SP34 (see Yang SJ, The Journal of Immunology (1986) 137; 1097-1100), TR-66 or X35-3, VIT3, BMA030 (BW264/56), CLB-T3/3, CRIS7, YTH12.5, F111-409, CLBT3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, and WT-31. Exemplary amino acid sequences of the anti-CD3 ABD are provided in FIG. 27.

In some embodiments, the anti-CD3 ABD has from 0, 1, 2, 3, 4, 5 or 6 amino acid modifications (with amino acid substitutions finding particular use). That is, the CDRs can be modified as long as the total number of changes in the set of 6 CDRs is less than 6 amino acid modifications, with any combination of CDRs being changed; e.g., there may be one amino acid change in v1CDR1, two in vhCDR2, none in vhCDR3, etc.).

In some embodiments, the anti-CD3 ABD is humanized or from human. For example, the anti-CD3 ABD can comprise a light chain variable region comprising human CDRs or non-human light chain CDRs in a human light chain framework region; and a heavy chain variable region comprising human or non-human heavy chain CDRs in a human heavy chain framework region. In some embodiments, the light chain framework region is a lamda light chain framework. In other embodiments, the light chain framework region is a kappa light chain framework.

In some embodiments, the anti-CD3 ABD is a single chain variable fragment (scFv) comprising a light chain variable region and a heavy chain variable region of an anti-CD3 antibody sequence provided herein. As used herein, “single chain variable fragment” or “scFv” refers to an antibody fragment comprising a variable region of a light chain and a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. The light chain variable region and heavy chain variable region of a scFv can be, e.g., in any of the following orientations: light chain variable region-scFv linker-heavy chain variable region or heavy chain variable region- scFv linker-light chain variable region.

Accordingly, in some embodiments, the anti-CD3 ABD is a single chain variable fragment (scFv) comprising a light chain variable region and a heavy chain variable region of an anti-CD3 antibody sequence provided herein. scFvs which bind to CD3 can be prepared according to known methods. For example, scFv molecules can be produced by linking VH and VL regions together using flexible polypeptide linkers. The scFv molecules comprise a scFv linker with an optimized length and/or amino acid composition. Accordingly, in some embodiments, the length of the scFv linker is between 10 to about 25 amino acids. Regarding the amino acid composition of the scFv linkers, peptides are selected that confer flexibility, do not interfere with the variable domains as well as allow inter-chain folding to bring the two variable domains together to form a functional CD3 binding site. In some embodiments, a scFv linker comprises glycine and serine residues. The amino acid sequence of the scFv linkers can be optimized, for example, by phage-display methods to improve the CD3 binding and production yield of the scFv. Examples of peptide scFv linkers suitable for linking a variable light chain region and a variable heavy chain region in a scFv include but are not limited to (GS)n (SEQ ID NO: 325), (GGS)n (SEQ ID NO: 326), (GGGS)n (SEQ ID NO:327), (GGSG)n (SEQ ID NO: 328), (GGSGG)n (SEQ ID NO: 329), or (GGGGS)n (SEQ ID NO: 330), wherein n is 1, 2, 3, 4, 5, 6, 7, 8, 9, or 10. In some embodiments, the peptide scFv linker is selected from GGGGSGGGGSGGGGS (SEQ ID NO: 312), GGGGSGGGGSGGGGSGGGGS (SEQ ID NO: 313), GGSGGSGGSGGSGG (SEQ ID NO: 318).

In some embodiments, the anti-CD3 antigen binding domain of a T cell engager domain has an affinity to CD3 on CD3 expressing cells with a KD of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. The affinity to bind to CD3 can be determined, for example, by Surface Plasmon Resonance (SPR).

### (ii) Tumor Associated Antigen Antigen Binding-Domain (“TAA-ABD”)

In some embodiments, the target antigen ABD of a T cell engager binds to a target antigen involved in and/or associated with a disease, disorder or condition, for example, a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. In some embodiments, a target antigen is a tumor associated antigen expressed on a tumor cell.

In some embodiments, a target antigen is a cell surface molecule such as a protein, lipid or polysaccharide. In some embodiments, a target antigen is on a tumor cell.

The target antigen binding-domain in this invention can take any format, including but not limited to a full antibody, an Fab, an Fv, a single chain variable fragments (scFv), a single domain antibody such as a heavy chain variable domain (VH), a light chain variable domain (VL) and a variable domain (VHH) of camelid derived single domain antibody.

In some embodiments, the target antigen is a tumor-associated antigen expressed on cancer cells. For example, the tumor-associate antigen is CD19, and the T cell engager domain targets cancer expressing CD19, such as most B cell malignancies including but not limited to acute lymphoblastic leukemia (ALL), chronic lymphocytic leukemia (CLL) and B cell lymphomas. Exemplary CD19 binding domain can include an antibody moiety derived from one or more CDRs of the anti-CD19 binding domain of Blinatumomab, SAR3419, MEDI-551, or Combotox.

### 3. Fusion Protein Moieties

In some embodiments, a fusion protein moiety (also referred to herein as “third monomers”) comprises, from N- to C-terminal, the second CID domain-domain linker-therapeutic moiety, and in additional embodiments the N- to C-terminal order is therapeutic moiety-domain linker-the second CID domain. Two halves of a CID are as described above, and any one of the two halves can be used to link with a therapeutic moiety to form a fusion protein moiety in this invention.

### a. Therapeutic Moieties

As discussed herein, the present invention is generally directed to the ability to control the half-life of therapeutic moieties in the blood stream of patients. Thus, while generally any therapeutic moiety can be used in the present invention, those with particular adverse side effects such as T cell engager drugs, find particular use in the present invention.

Any therapeutic moiety can be used to link with the second CID domain to create the fusion protein moiety described herein. For example, a therapeutic moiety includes, but is not limited to, a T cell engager moiety; an antibody including, but not limited to an antibody fragment taking various formats such as an Fv, an scFv, and a single domain antibody (sdAb; including fragments such as the VHH domain of a camelid derived sdAb); a cytokine; a hormone; a peptide; an antibody drug conjugate; or a peptide drug conjugate.

In some embodiments, the therapeutic moiety is an antibody or antibody fragment targeting an antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. In some embodiments, the therapeutic moiety is an antibody or antibody fragment binding one or more tumor-associated antigens expressed on a tumor cell as described herein.

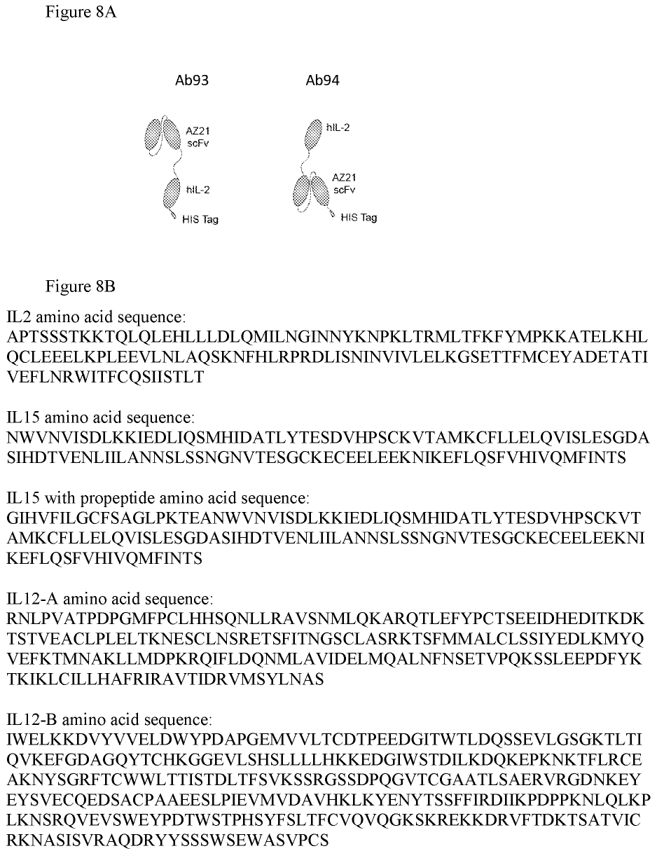

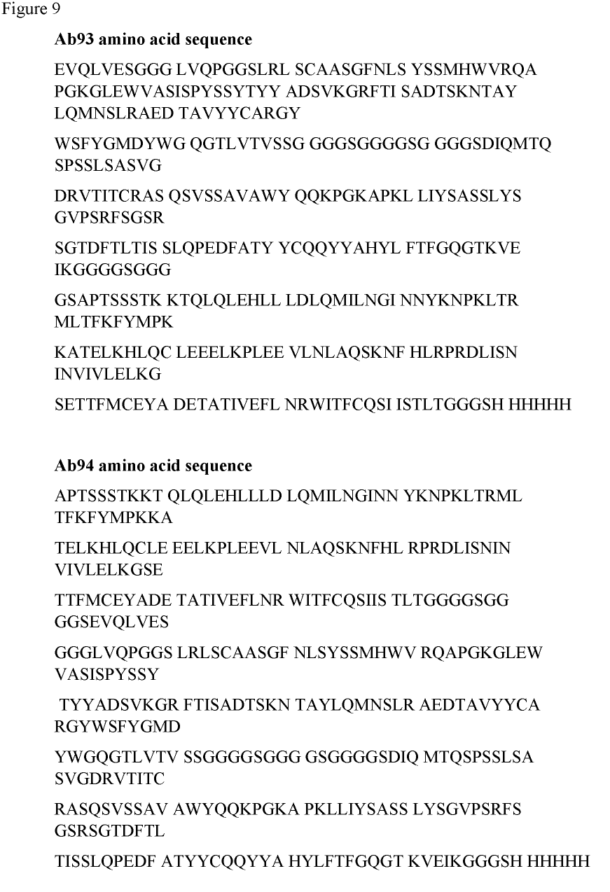

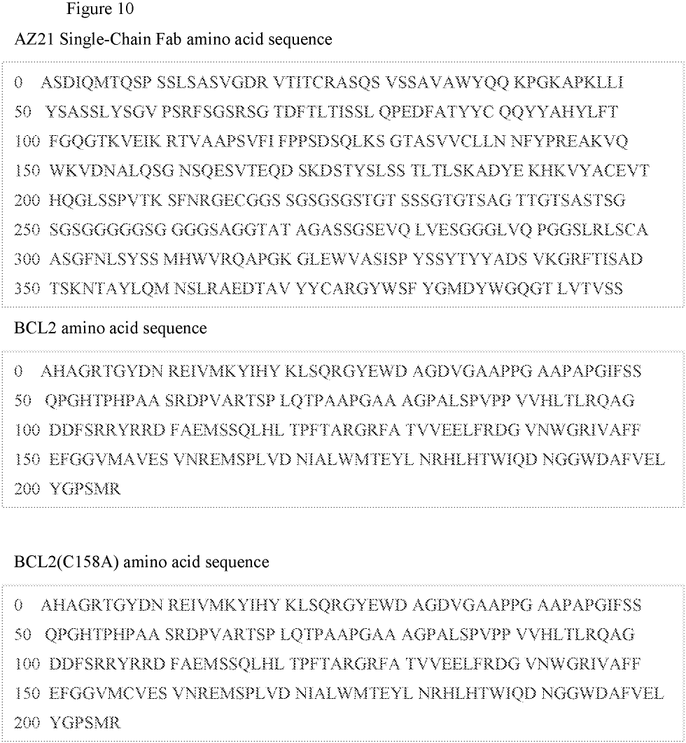

In some embodiments, the therapeutic moiety is an interleukin molecule as generally shown in FIG. 8, such as but not limited to an IL-2, IL-12, IL-15, and variants thereof.

### (i) T Cell Engager Domains

In particularly useful embodiments, the therapeutic moiety is a T cell engager moiety. As is known in the art, while these are generally very effective cancer therapeutic agents, they also can exhibit toxic side effects, and thus the ability to rapidly remove them from a patient’s blood stream is extremely beneficial.

In general, as is known in the art, a T cell engager moiety comprises a T cell antigen binding domain (TC-ABD) and a tumor target associated antigen binding domain (TTA-ABD), and is designed to allow specific targeting of cells expressing the target antigen by recruiting cytotoxic T cells. For example, a T cell engager moiety described herein can engage cytotoxic T cells via binding to the surface-expressed CD3 proteins, which form part of the T cell receptor (TCR). Simultaneous binding of a T cell engager moiety to CD3 and to a target antigen expressed on the surface of particular cells causes T cell activation and mediates the subsequent lysis of the particular target antigen-expressing cell. Thus, a T cell engager can induce strong, specific and efficient target cell killing.

In some embodiments, a T cell engager moiety described herein comprises a T cell ABD and a target antigen-binding domain, wherein the target antigen is expressed on pathogenic cells (e.g., tumor cells, virally or bacterially infected cells, autoreactive T cells, etc). As a result, the T cell engager stimulates target cell killing by cytotoxic T cells to eliminate the pathogenic cells. Exemplary T cell engagers are described in Dreier, T. et al., Int. J. Cancer, 100: 690-697 (2002); and Brischwein K et al., Molecular Immunology Vol 43, Issue 8, 1129-1243 (2006), both of which are entirely incorporated by reference.

In some embodiments, the C terminus of a T cell ABD is linked to the N terminus of a target ABD, via a domain linker. In other embodiments, the N terminus of a T cell binding-domain is linked to the C terminus of a target antigen-binding domain of a T cell engager via a domain linker.

In some embodiments, including those depicted in FIG. 3, the therapeutic moiety that is a T cell engager moiety is actually split between two monomers, with the T cell ABD and the tumor antigen ABD on different chains. This is generally discussed below.

### (a) T Cell ABD

The binding specificity of a T cell engager moiety to T cells is mediated by the recognition of the TCR. As part of the TCR, CD3 is a protein complex that includes a CD3λ (gamma) chain, a CD3b (delta) chain, and two CD3ε (epsilon) chains which are present on the cell surface. CD3 associates with the α (alpha) and β (beta) chains of the TCR as well as CD3 (zeta) altogether to form the complete TCR. Clustering of CD3 on T cells, such as by immobilized anti-CD3 antibodies, leads to T cell activation similar to the engagement of the T cell receptor but independent of its clone typical specificity.

The T cell engager moiety described herein comprises a domain which specifically binds to the TCR. In some embodiments, the T cell engager moiety described herein comprises a domain which specifically binds to human CD3ε.

In some embodiments, the T cell ABD of the T cell engager moiety comprises an antigen binding-domain derived from a monoclonal antibody, a polyclonal antibody, a recombinant antibody, a human antibody, or a humanized antibody. The T cell ABD can take any format, including but not limited to an Fv, an scFv, and an sdAb such as the VHH domain of a camelid derived sdAb.

In some embodiments, the T cell ABD of the T cell engager moiety is an anti-CD3 ABD, which comprises a set of three light chain CDRs (v1CDR1, vlCDR2 and vlCDR3), and three heavy chain CDRs (vhCDR1, vhCDR2 and vhCDR3) of an anti-CD3 antibody. Exemplary anti-CD3 antibodies to contribute the CDR sets, including, but not limited to, L2K, UCHT1, variants of UCHT1 including UCHT1.v9, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLBT3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, and WT-31. Exemplary amino acid sequences of the anti-CD3 ABD are provided in FIG. 27.

In some embodiments, the anti-CD3 ABD has from 0, 1, 2, 3, 4, 5 or 6 amino acid modifications (with amino acid substitutions finding particular use). That is, the CDRs can be modified as long as the total number of changes in the set of 6 CDRs is less than 6 amino acid modifications, with any combination of CDRs being changed; e.g., there may be one amino acid change in v1CDR1, two in vhCDR2, none in vhCDR3, etc.

In some embodiments, the anti-CD3 ABD is humanized or from human. For example, the anti-CD3 ABD can comprise a light chain variable region comprising human CDRs or non-human light chain CDRs in a human light chain framework region; and a heavy chain variable region comprising human or non-human heavy chain CDRs in a human heavy chain framework region. In some embodiments, the light chain framework region is a lamda light chain framework. In other embodiments, the light chain framework region is a kappa light chain framework.

In some embodiments, the anti-CD3 ABD is a single chain variable fragment (scFv) comprising a light chain variable region and a heavy chain variable region of an anti-CD3 antibody sequence provided herein. scFvs which bind to CD3 can be prepared according to known methods. For example, scFv molecules can be produced by linking VH and VL regions together using flexible polypeptide linkers. The scFv molecules comprise a scFv linker with an optimized length and/or amino acid composition. Accordingly, in some embodiments, the length of the scFv linker is between 10 to about 25 amino acids. Regarding the amino acid composition of the scFv linkers, peptides are selected that confer flexibility, do not interfere with the variable domains as well as allow inter-chain folding to bring the two variable domains together to form a functional CD3 binding site. In some embodiments, a scFv linker comprises glycine and serine residues. The amino acid sequence of the scFv linkers can be optimized, for example, by phage-display methods to improve the CD3 binding and production yield of the scFv. Examples of peptide scFv linkers suitable for linking a variable light chain region and a variable heavy chain region in a scFv include but are not limited to (GS)n (SEQ ID NO: 325), (GGS)n (SEQ ID NO: 326), (GGGS)n (SEQ ID NO:327), (GGSG)n (SEQ ID NO: 328), (GGSGG)n (SEQ ID NO: 329), or (GGGGS)n (SEQ ID NO: 330), wherein n is 1, 2, 3, 4, 5, 6, 7, 8, 9, or 10. In some embodiments, the peptide scFv linker is selected from GGGGSGGGGSGGGGS (SEQ ID NO: 312), GGGGSGGGGSGGGGSGGGGS (SEQ ID NO: 313), GGSGGSGGSGGSGG (SEQ ID NO: 318).

In some embodiments, the anti-CD3 antigen binding domain of a T cell engager moiety has an affinity to CD3 on CD3 expressing cells with a KD of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. The affinity to bind to CD3 can be determined, for example, by Surface Plasmon Resonance (SPR).

### (b) Target ABD

In addition to the component that binds to T cells, e.g. an anti-CD3 ABD (CD3-ABD), the T cell engager moiety also includes an ABD that binds to a target antigen, generally a target tumor-associated antigen (TTA), linked through a domain linker as described above. Thus, in some embodiments, the target antigen ABD of a T cell engager moiety binds to a target antigen involved in and/or associated with a disease, disorder or condition, for example, a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. In some embodiments, a target antigen is a cell surface molecule such as a protein, lipid or polysaccharide. In some embodiments, a target antigen is a tumor-associated antigen expressed on a tumor cell.

The target antigen ABD in this invention can take any format, including but not limited to a full antibody, an Fab, an Fv, a single chain variable fragments (scFv), a single domain antibody such as the VHH of camelid derived single domain antibody. In many embodiments, the target antigen ABD is a scFv.

In some embodiments, the target antigen is a tumor-associated antigen expressed on cancer cells. For example, the tumor-associate antigen is CD19, and the T cell engager moiety targets cancer expressing CD19, such as most B cell malignancies including but not limited to acute lymphoblastic leukemia (ALL), chronic lymphocytic leukemia (CLL) and B cell lymphomas. Exemplary CD19 binding domain can include an antibody moiety derived from one or more CDRs of the anti-CD19 binding domain of Blinatumomab, SAR3419, MEDI-551, or Combotox.

In addition to CD19, any other tumor-associated-antigen is envisioned, such as Her2.

### B. Compositions Comprising ClnD

As discussed herein, the compositions of the invention rely on one of two mechanisms: either the monomer components are held together with a small molecule for function (the “CID embodiments” as described above) or the associated monomers are separated by the addition of a small molecule (the “CInD embodiments”).

Accordingly, another aspect of the invention involves a composition comprising a dimeric Fc fusion protein and a fusion protein moiety. The dimeric Fc fusion protein comprises a first monomer containing a first Fc domain linked to one half of a chemically inhibited dimerizer (CInD), referred herein as “a first CInD domain”, optionally via a domain linker; and a second monomer containing a second Fc domain that dimerizes with the first Fc domain. The fusion protein moiety comprises a therapeutic moiety linked to the other half of the CInD domain, referred herein as “a second CInD domain”, optionally via a domain linker. After administration, the two halves of the CInD domains form a dimer, enabling association of the therapeutic moiety with the Fc domain, and extending the serum half-life of the therapeutic moiety. In the event that the therapeutic moiety needs to be cleared from the blood quickly, a CInD small molecule is administered, which induces disassociation of the two halves of the CInD, thereby enabling disassociation of the therapeutic moiety from the Fc domain, and clearance of the therapeutic moiety in the patient.

In some embodiments, the Fc fusion protein is heterodimeric with one monomer containing a first CInD domain linked to a first Fc domain, and the other monomer containing an an Fc domain alone (e.g., an “empty Fc domain,”), as illustrated in FIG. 2A. The first and second Fc domain heterodimerize, for example, by incorporating the heterodimerization mutations described herein.

In some embodiments, the Fc fusion protein is heterodimeric with one monomer containing a first CInD domain linked to a first Fc domain, and the other monomer containing a second Fc domain linked to a second therapeutic moiety, optionally via a linker, as illustrated in FIG. 3. Administration of the heterodimeric Fc fusion protein with the fusion protein moiety comprising a therapeutic moiety linked to a second CInD domain described above induces the association of the two halves of the CInD domains, enabling association of the therapeutic moiety with the Fc domain, and extending the serum half-life of the therapeutic moiety. In addition, this format imparts a bispecific nature to the therapeutic moieties and can increase the potency of the therapeutic moieties while simultaneously increasing their serum half-life. The first and second Fc domains heterodimerize, for example, by incorporating the heterodimerization mutations described herein.

In some other embodiments, the Fc fusion protein is homodimeric with two identical monomers, each containing a first CInD domain linked to a Fc domain, optionally via a linker, as illustrated in FIG. 4. Administration of the homodimeric Fc fusion protein with the fusion protein moiety comprising a therapeutic moiety linked to a second CInD domain described above induces the association of the two halves of the CInD domains, enabling association of the therapeutic moiety with the Fc domains, and extending the serum half-life of the therapeutic moiety. This format increases the stoichiometry and valancing of the therapeutic moiety while simultaneously extending its half-life.

### 1. Heterodimeric Fc Fusion Proteins

For the heterodimeric Fc fusion proteins that comprise a first CInD domain, the Fc domains which heterodimerise with each other are generally described herein. Additional Fc variants including, but not limited to, Fc ablation variants, FcRn variants, FcyR variants and/or half life extension variants can also be introduced in combination with the heterodimerization mutations as generally outlined herein.

In some embodiments, the heterodimeric Fc fusion protein comprises a first therapeutic moiety linked to the second monomer, optionally via a linker. The first therapeutic moiety can be an antibody; an antibody fragment taking various formats such as but not limited to an Fab, an Fv, an scFv, a single domain antibody such as the VHH of camelid derived single domain antibody; a cytokine; a hormone; a peptide; an antibody drug conjugate; or a peptide drug conjugate. In some embodiments, the first therapeutic moiety is an antibody or antibody fragment targeting an antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease.

In some embodiments, the first therapeutic moiety is an antibody or antibody fragment binding one or more tumor-associated antigens expressed on a tumor cell as described herein.

In some embodiments, the first therapeutic moiety is an interleukin molecule, such as but not limited to an IL-2, IL-12, and IL-15.

In some embodiments, the first therapeutic moiety works together with a second therapeutic moiety of the fusion protein moiety to act as a bispecific molecule, binding to two targets. In some embodiments, the two targets are located on the same cell. In some embodiments, the two targets are located on different cells. In some embodiments, one target is located on a cell, and the other target is located in the microenvironment where the cell resides.

For example, the first therapeutic moiety can work together with the second therapeutic moiety within the fusion protein moiety to act as T cell engager, wherein the first therapeutic moiety being an ABD recognizing a T cell antigen, such as a CD3 ABD; and the second therapeutic moiety being an ABD recognizing a target antigen involved in and/or associated with a disease, disorder or condition, for example, a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. Alternatively, the second therapeutic moiety can be an ABD recognizing a T cell antigen, such as a CD3 ABD; and the first therapeutic moiety can be an ABD recognizing a target antigen involved in and/or associated with a disease, disorder or condition, for example, a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. In some embodiments, the target antigen is a cell surface molecule such as a protein, lipid or polysaccharide. In some embodiments, the target antigen is a tumor-associated antigen expressed on a tumor cell described herein, such as CD19.

### a. ClnD

Chemically inhibited dimerization is a biological mechanism in which two proteins non-covalently associate or bind, and the association is disrupted by a small molecule. In the present invention, the disruptive small molecule is referred to as a “Chemically Inhibited Dimerizer (CInD) small molecule” or a “CInD small molecule” or “CInDSM”.

In the present invention, two CInD domains come in pairs that will be disassociate in the presence of a CInDSM. As will be appreciated by those in the art, some CInD pairs are identical, e.g., both of the CInD domains are identical. In other embodiments, the CInD pairs are made up of two different CInD domains.

Any CInD pairs can be used in the present invention. In some embodiments of the present invention, a CInD pair is derived from naturally occurring binding partners. In some embodiments, a CInD pair comprises a protein and an antigen binding domain (ABD) that binds specifically to the protein. In some embodiments, a CInD pair comprises two antibody moieties, wherein one acts as an antigen and the other acts as an antibody.

The CInD small molecule can be naturally occurring and disrupt the association of the CInD pair. The CInD small molecule can also be screened out from a small molecule library that can disrupt the pairing of the CInD pair. In some embodiments, the CInD small molecule disrupts the CInD pair by binding to one domain of the CInD pair with at least 2-fold higher affinity than the binding affinity of the two domains of the CInD pair, e.g., at least 2 fold, at least 5 fold, at least 10 fold, at least 20 fold, at least 50 fold, at least 100 fold. In some embodiments, the CInD small molecule disrupts the CInD pair by masking or changing the binding interface between the two domains of the CInD pair.

CInD domain pairs and CInDSM can be produced using a screening performed from an antibody library, a DARPin library, a nanobody library, or an aptamer library or a phage displayed Fab library. For example, as the step 1, binding moieties can be selected that bind to the cognate binding moiety in the absence of the small molecule, thereby generating a set of selected binding moieties; and then, as step 2, the selected binding moieties can be screened for binding moieties that do not bind the cognate binding moiety in the presence of a small molecule, thereby generating a set of counter selected binding moieties. Steps 1 and 2 of screening can be conducted one or more rounds, wherein each round of screening comprises the screening of step 1 and the screening of step 2, such that a set of binding moieties that specifically dissociate from the cognate binding moiety in the presence of the small molecule is generated.

### 2. Homodimeric Fc Fusion Proteins

### a. Fc Domains

In one aspect, the dimeric Fc fusion protein is a homodimeric Fc fusion protein. Such homodimeric Fc fusion proteins include a first monomer and a second monomer each having an Fc domain and a first CInD domain with the same amino acid sequence. In some embodiments, the Fc domain is linked to a first CInD domain via a domain linker, which is generally described herein. In some embodiments, the Fc domain linked to a first CInD domain without a domain linker. These Fc domains are generally described above, but lack the heterodimerization variants.

### 3. Fusion Protein Moieties

Fusion protein moieties comprise a second CInD domain and a therapeutic moiety. The second CInD domain and the therapeutic moiety are as generally described herein.

### C. Exemplary Compositions

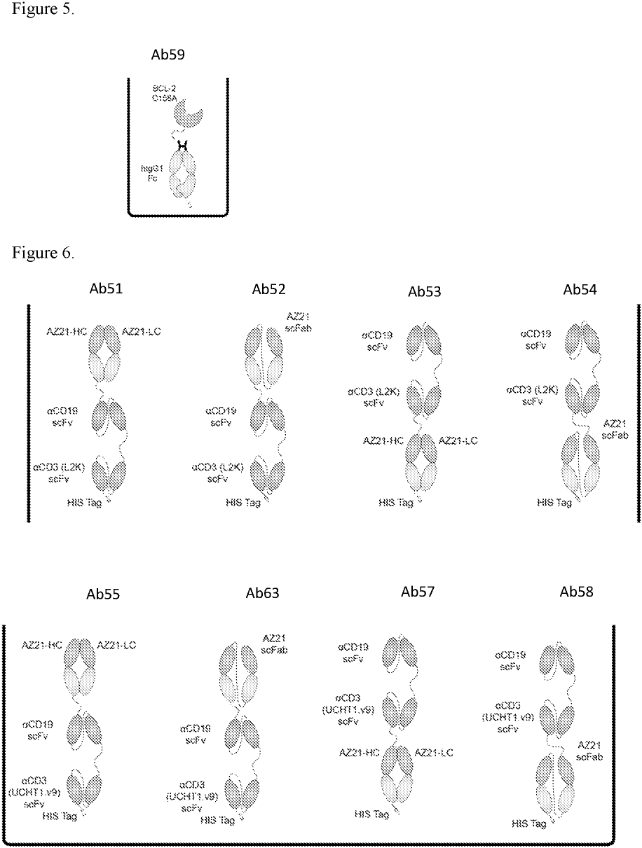

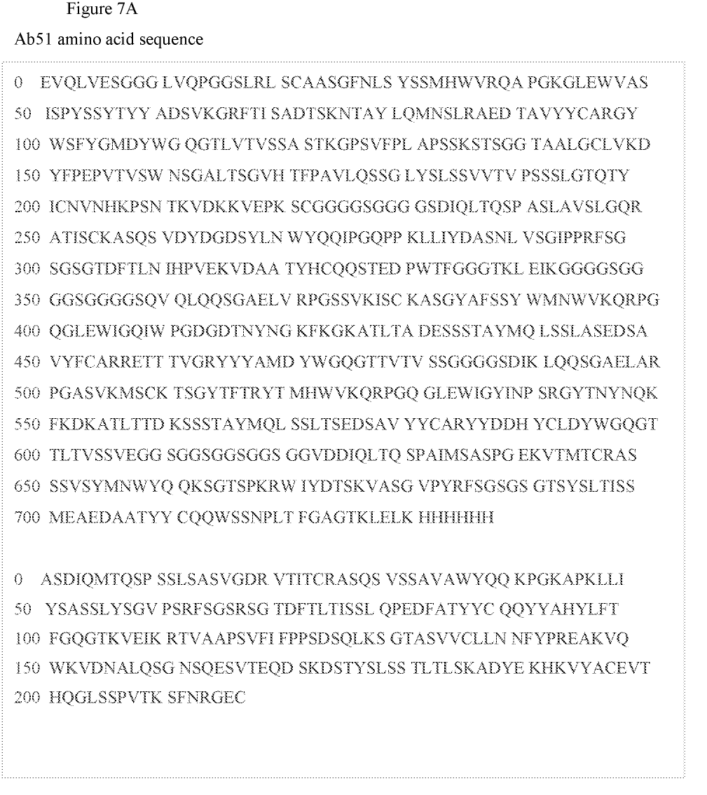

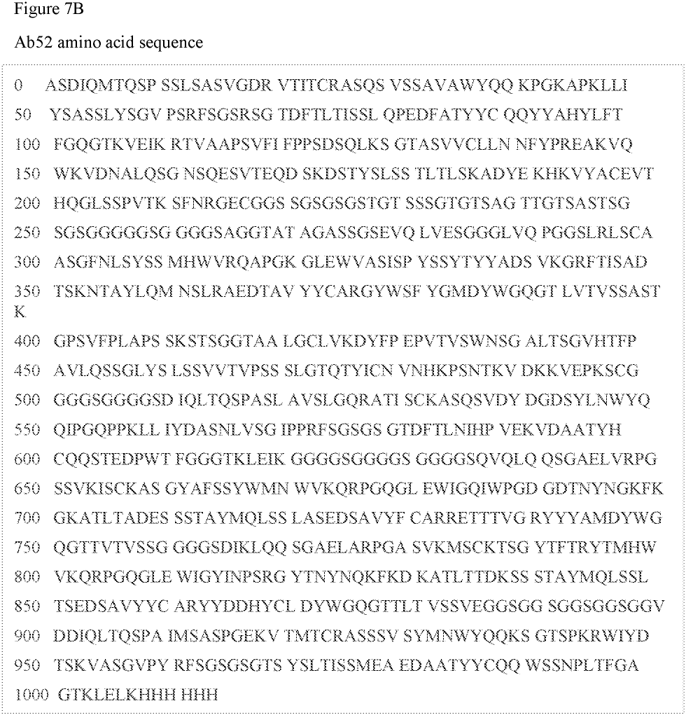

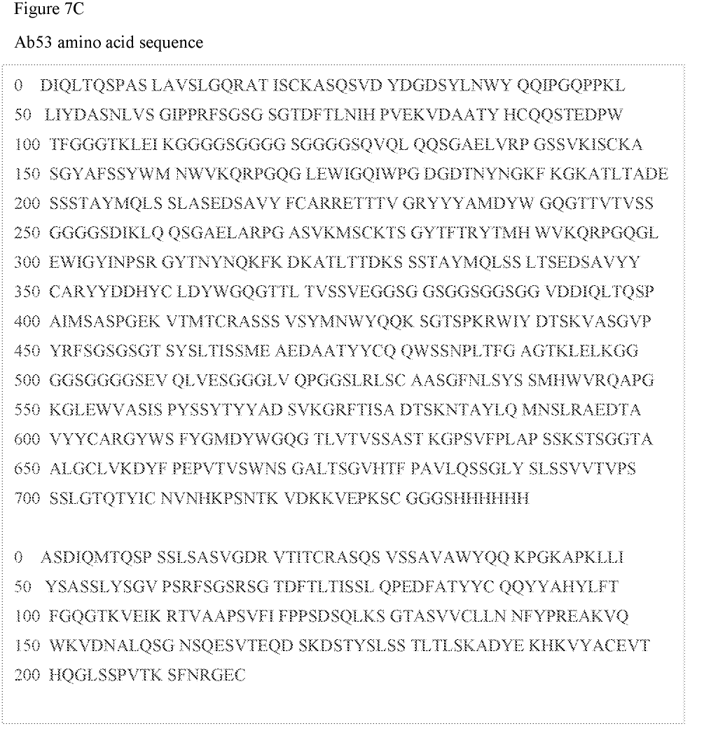

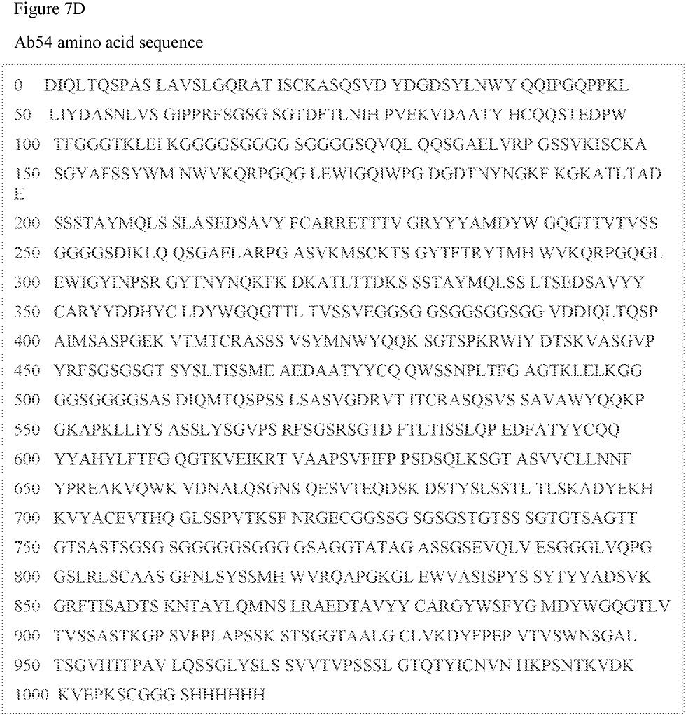

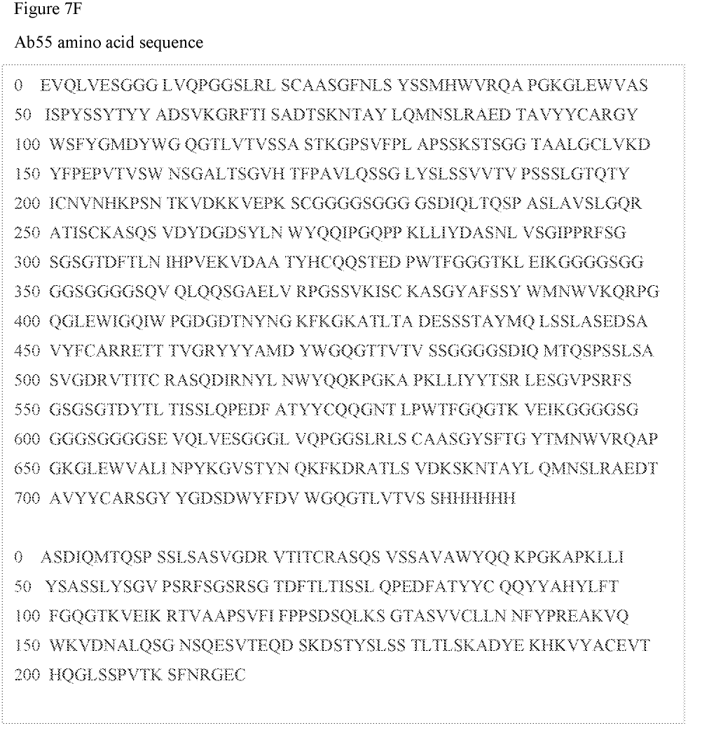

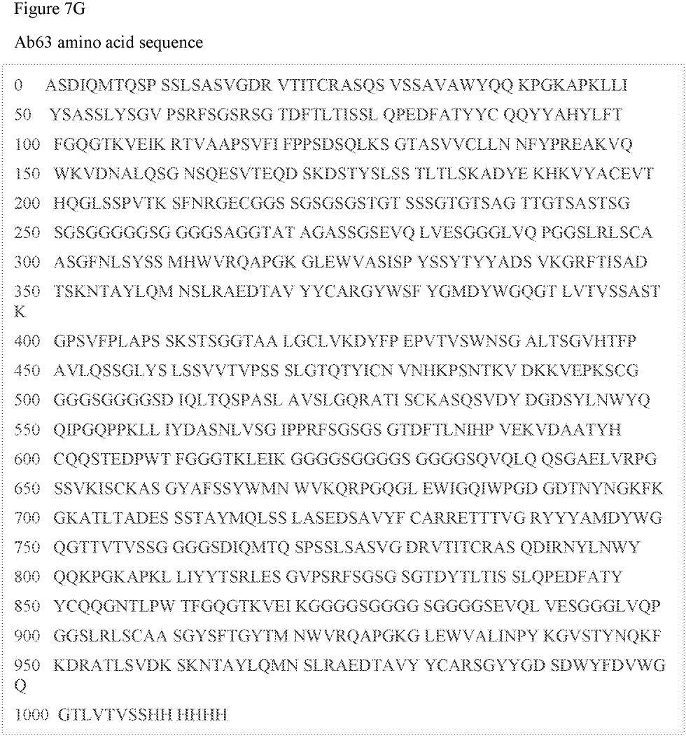

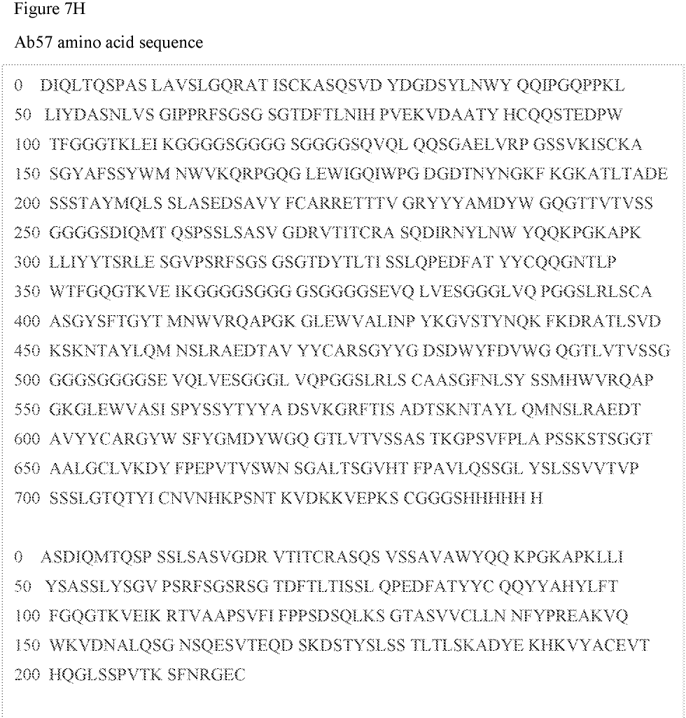

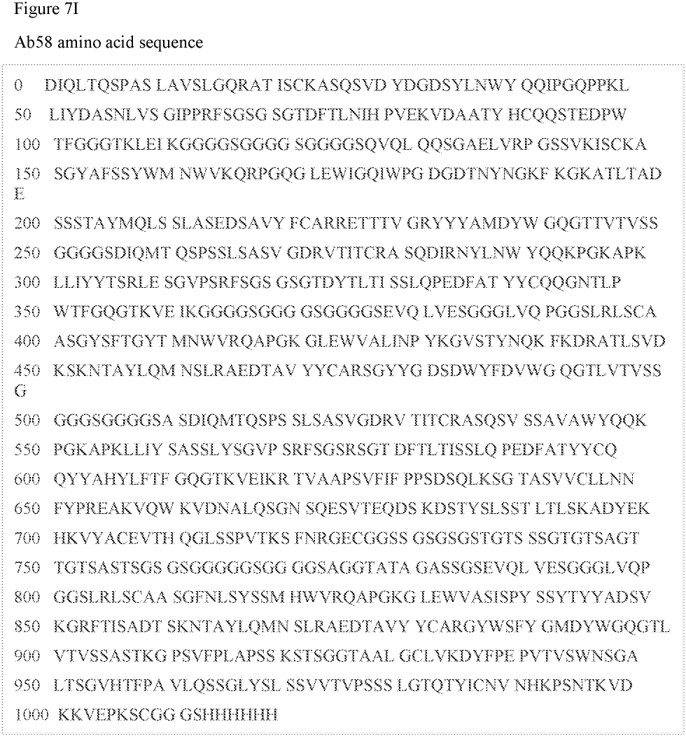

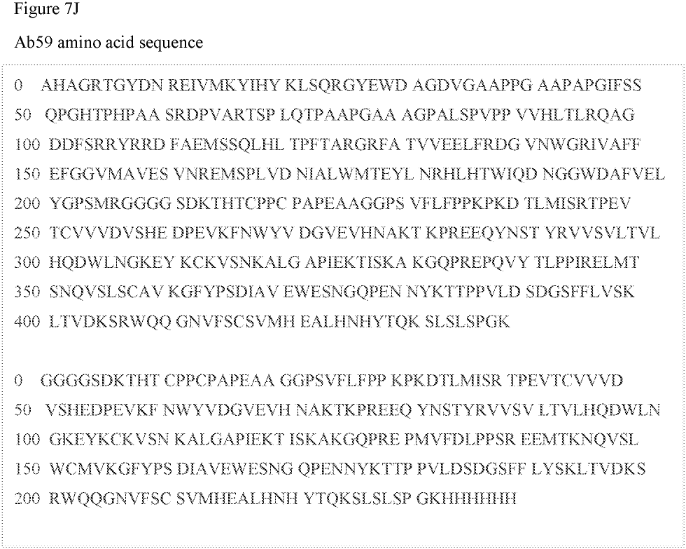

In some embodiments, the invention involves a composition comprising a heterodimeric Fc fusion protein comprising a first CID domain and a fusion protein moiety comprising a second CID domain and a therapeutic domain illustrated in FIG. 1B. The heterodimeric Fc fusion protein is fused by a first and a second monomer. The first monomer comprises a first CID domain BCL-2 or BCL-2 (C158A) covalently linked to a human IgG1 Fc domain, via a domain linker. The first monomer comprises, from N- to C-terminal, BCL-2 or BCL-2 (C158A)-domain linker-human IgG1 Fc domain, or from N- to C-terminal, human IgG1 Fc domain-domain linker-BCL-2 or BCL-2 (C158A). The second monomer comprises an empty human IgG1 Fc that dimerizes with the Fc domain of the first monomer. The fusion protein moiety comprises a second CID domain AZ21 linked to a T cell engager comprising a CD3 scFv and a CD19 scFv, via a domain linker. In some embodiments, the fusion protein moiety comprises, from N- to C-terminal, AZ21-domain linker-CD19 scFv-CD3 scFv. In some embodiments, the fusion protein moiety comprises, from N- to C-terminal, CD19 scFv-CD3 scFv-domain linker-AZ21. AZ21 can be formatted into a Fab or single chain Fab. Different configurations of the composition are depicted in FIG. 5 and FIG. 6 as Ab59, Ab51, Ab52, Ab53, Ab54, Ab55, Ab63, Ab57, and Ab58. Addition of a CID small molecule ABT199 induces association of BCl-2 or BCL-2 (C158A) with AZ21, thereby enabling association of the T cell engager with the Fc domain, and extending the serum half-life of the T cell engager.

In some embodiments, the invention involves a composition comprising a heterodimeric Fc fusion protein comprising a first CInD domain and a fusion protein moiety comprising a second CInD domain and a therapeutic domain illustrated in FIG. 2B. The heterodimeric Fc fusion protein is fused by a first and a second monomer. The first monomer comprises a first CInD domain covalently linked to a human IgG1 Fc domain, via a domain linker. The first monomer comprises, from N- to C-terminal, first CInD domain-domain linker-human IgG1 Fc domain, or from N- to C-terminal, human IgG1 Fc domain-domain linker-first CInD domain. The second monomer comprises an empty human IgG1 Fc that dimerizes with the Fc domain of the first monomer. The fusion protein moiety comprises a second CInD domain linked to a T cell engager comprising a CD3 scFv and a CD19 scFv, via a domain linker. In some embodiments, the fusion protein moiety comprises, from N- to C-terminal, second CInD domain-domain linker-CD19 scFv-CD3 scFv. In some embodiments, the fusion protein moiety comprises, from N- to C-terminal, CD19 scFv-CD3 scFv-domain linker-second CInD domain. The first CInD domain and the second CInD domain associate to form dimer, bringing the T cell engager to association with Fc domain and thereby extending the serum half-life of the T cell engager. In the event that the patient needs to have the therapeutic moiety cleared quickly, for example, due to safety concerns, the patient would be dosed with the CInD small molecule, which disrupts the CInD pairs. This leads to dissociation of the T cell engager from the Fc domain and rapid clearance of the T cell engager in the patient.

In some embodiments, the invention involves a composition comprising a heterodimeric Fc fusion protein and a fusion protein moiety as illustrated in FIG. 3. The heterodimeric Fc fusion protein comprises a first monomer containing a first CInD domain linked to a first human IgG1 Fc domain via a linker, and a second monomer containing a second human IgG1 Fc domain linked to a second therapeutic moiety such a CD19 scFv via a linker. The first IgG1 Fc domain that dimerizes with second Fc domain. The fusion protein moiety comprises a second CInD domain linked to a therapeutic moiety such as a CD3 scFv. Association of two CInD domains enables association of the therapeutic moieties with the Fc domain, and extending the serum half-life of the therapeutic moieties. In addition, this format brings the therapeutic moieties together and imparts a bispecific nature to the therapeutic moieties (such as bringing CD3 ABD and CD19 ABD together as a T cell engager), increasing the potency while simultaneously increasing their serum half-life. CD3 scFv and CD19 scFv can swap position within the composition, and they can be linked to the neighboring domain either at their N or C terminus. In the event that the patient needs to have the therapeutic moieties cleared quickly, for example, due to safety concerns, the patient would be dosed with the CInD small molecule, which disrupts the CInD pairs. This leads to dissociation of one therapeutic moiety from the Fc domain and rapid clearance of the therapeutic moiety in the patient.

In some embodiments, the invention involves a composition comprising a homodimeric Fc fusion protein and a fusion protein moiety as illustrated in FIG. 4. The Fc fusion protein comprises two identical monomers, each containing a first CInD domain linked to a human IgG1 Fc domain via a linker. The fusion protein moiety comprises a second CInD domain linked to a therapeutic moiety via a domain linker. For example, the therapeutic moiety can be a T cell engager comprising a CD19 scFv and a CD3 scFv. Association of two CInD domains enables association of the therapeutic moieties with the Fc domain, and extending the serum half-life of the therapeutic moieties. This format enables the association of two therapeutic moieties with a single Fc dimer, which leads to increased stoichiometry and can increase potency of the therapeutic moieties while extending their serum half-life. In the event that the patient needs to have the therapeutic moieties cleared quickly, for example, due to safety concerns, the patient would be dosed with the CInD small molecule, which disrupts the CInD pairs. This leads to dissociation of the therapeutic moieties from the Fc domain and rapid clearance of the therapeutic moieties in the patient.

### D. Nucleic Acids Encoding the Composition

Nucleic acid compositions encoding the composition described herein are provided, including polynucleotide molecules encoding the monomer components of the invention. That is, generally the compositions of the invention comprise three monomers (two Fc fusion protein monomers and a fusion protein moiety), each of which are encoded by nucleic acids.

Expression vectors containing the nucleic acids, and host cells transformed with the nucleic acids and/or expression vectors are also provided. As will be appreciated by those in the art, the protein sequences depicted herein can be encoded by any number of possible nucleic acid sequences, due to the degeneracy of the genetic code.

In some embodiments, the polynucleotide molecules are provided as DNA constructs.

In some embodiments, the polynucleotide molecules encoding each monomer of the dimeric Fc fusion proteins and a fusion protein moiety are placed into a single expression vector. In some embodiments, the polynucleotide molecules encoding each monomer of the dimeric Fc fusion proteins and a fusion protein moiety are placed into different expression vectors. Expression vectors, as is known in the art, can contain the appropriate transcriptional and translational control sequences, including, but not limited to, signal and secretion sequences, regulatory sequences, promoters, origins of replication, selection genes, etc.

Expression vectors can be transformed into host cells, where they are expressed to form the composition described herein. An appropriate host cell expression system includes but is not limited to bacteria, an insect cell, and a mammalian cell. Preferred mammalian host cells for expressing the recombinant antibodies according to at least some embodiments of the invention include Chinese Hamster Ovary (CHO cells), PER.C6, HEK293 and others as is known in the art.

In some embodiments, the composition described herein is produced by introducing one or more expression vectors expressing the composition into a host cell and culturing said host cell under conditions whereby the proteins are expressed, may be isolated and, optionally, further purified.

### E. Formulations

The compositions used in the practice of the foregoing methods can be formulated into pharmaceutical compositions comprising a carrier suitable for the desired delivery method. Suitable carriers include any material that when combined with the therapeutic composition retains the therapeutic function of the therapeutic composition and is generally non-reactive with the patient’s immune system. Examples include, but are not limited to, any of a number of standard pharmaceutical carriers such as sterile phosphate buffered saline solutions, bacteriostatic water, and the like (see, generally, Remington’s Pharmaceutical Sciences 16th Edition, A. Osal., Ed., 1980). Acceptable carriers, excipients, or stabilizers are nontoxic to recipients at the dosages and concentrations employed and may include buffers.

Administration of the pharmaceutical compositions described in the present invention, preferably in the form of a sterile aqueous solution, may be done in a variety of ways, including, but not limited to intravenously or locally.

### F. Methods of Using the Composition

### 1. Fc Fusion Proteins

The compositions described herein can find use in a number of therapeutic applications. Usually, a patient is a human, but non-human mammals including transgenic mammals can also be treated.

The composition comprising a heterodimeric Fc fusion protein containing a first CID domain and a fusion protein moiety containing a second CID domain and a therapeutic moiety can be administered to a patient. Administration of a CID small molecule to the same patient induces association of the two CID domains, bringing the therapeutic moiety to association with Fc domain and thereby extending the serum half-life of the therapeutic moiety. The CID small molecule can be administered before, simultaneously with, or after the administration of the composition.

As will be appreciated by those in the art, the starting serum half-life of the therapeutic moiety can vary widely, with some moieties, like IL-2, having half-lives measured in hours, and others, like antibodies, have half-lives measured in days. Thus, serum half-life of the therapeutic moiety can be extended to at least about 12 hours, at least about about 1 day, at least about about 2 days, at least about 4 days, at least about 6 days, at least about 8 days, at least about 10 days, at least about 12 days, or at least about 14 days. Alternatively, the serum half-life can be extended by 1, 2, 3, 4, 5, 6, 7, 8, 9 or 10-fold, or in some cases from 1 to 100 fold. To maintain the association of the therapeutic moiety with Fc domain, the patient can be dosed regularly with the CID small molecule, and the frequency of dosing depends on combination of the CID small molecule’s serum half-life, and the lifetime of the CID complex.

In the event that the patient needs to have the therapeutic moiety cleared quickly, for example, due to safety concerns, the patient would stop being dosed with the CID small molecule, leading to clearance of the CID small molecule, disassociation of the therapeutic moiety from Fc domain, and rapid clearance of the therapeutic moiety in the patient. The rate of clearance of the therapeutic moiety depends on a combination of the CID small molecule’s serum half-life, the binding affinity of the CID small molecule to the first and second CID domains, the lifetime of the CID complex, and the clearance rate of the therapeutic moiety when no longer associated with Fc domain.

The composition comprising a heterodimeric or homodimeric Fc fusion protein containing a first CInD domain and a fusion protein moiety containing a second CInD domain and a therapeutic moiety can be administered to a patient. The first CInD domain and the second CInD domain associate to form dimer, bringing the therapeutic moiety to association with Fc domain and thereby extending the serum half-life of the therapeutic moiety.

As discussed above, serum half-life of the therapeutic moiety can be extended to at least about 12 hours, at least about 1 day, at least about 2 days, at least about 4 days, at least about 6 days, at least about 8 days, at least about 10 days, at least about 12 days, or at least about 14 days. Alternatively, the serum half-life can be extended by 1, 2, 3, 4, 5, 6, 7, 8, 9 or 10-fold, or in some cases from 1 to 100 fold.

In the event that the patient needs to have the therapeutic moiety cleared quickly, for example, due to safety concerns, the patient would be dosed with the CInD small molecule, which disrupts the CInD pairs. This leads to dissociation of the therapeutic moiety from the Fc domain and rapid clearance of the therapeutic moiety in the patient. The rate of clearance of the therapeutic moiety depends on the binding affinity of the CInD small molecule to the first and second CInD domains, and the clearance rate of the therapeutic moiety when no longer associated with Fc domain.

The methods described above enable a precise temporal control of the serum half-life of a therapeutic moiety in a patient, and the method is applicable to patients suffering from a variety of diseases or conditions, for example, a proliferative disease, a tumorous disease, an inflammatory disease, an immunological disorder, an autoimmune disease, an infectious disease, a viral disease, an allergic reaction, a parasitic reaction, a graft-versus-host disease or a host-versus graft disease. Depending on the disease of a patient and proper therapeutic moiety can be designed to be incorporated in the compositions described herein. For example, to treat and control the serum half-life of the therapeutic moiety in patients suffering from most of B cell malignancies including but not limited to acute lymphoblastic leukemia (ALL), chronic lymphocytic leukemia (CLL) and B cell lymphomas, a therapeutic moiety can incorporate a T cell engager comprising a CD19 ABD and a CD3 ABD into the compositions described herein.

Administration of the compositions described herein may be done in a variety of ways, including, but not limited to intravenously or locally.

The dosing amounts and frequencies of administration are, in a preferred embodiment, selected to be therapeutically or prophylactically effective. As is known in the art, dosages for any one patient depends on many factors, the age, body weight, general health, sex, diet, time and route of administration, drug interaction and the severity of the condition may be necessary, and will be ascertainable with routine experimentation by those skilled in the art.

### 2. HSA

The composition comprising the first and second monomers described herein can find use in a number of therapeutic applications. Usually, a patient is a human, but non-human mammals including transgenic mammals can also be treated.

In some embodiments, the composition is administered to a patient to extend the serum half-life of a T cell engager domain in the patient, wherein the T cell engager is used in the patient to stimulate target cell killing by cytotoxic T cells. The target cells are involved in and/or associated with a disease, disorder or condition, for example, a proliferative disease, and a tumorous disease. Administration of a CID small molecule to the same patient induces association of the first and second monomers, bringing the T cell engager domain to association with HSA and thereby extending the serum half-life of the T cell engager domain. The small molecule can be administered before, simultaneously with, or after the administration of the composition.

Serum half-life of the T cell engager domain can be extended to at least about 12 hours, at least about 1 day, at least about 2 days, at least about 4 days, at least about 6 days, at least about 8 days, at least about 10 days, at least about 12 days, or at least about 14 days. Alternatively, the serum half-life can be extended by 1, 2, 3, 4, 5, 6, 7, 8, 9 or 10-fold, or in some cases from 1 to 100 fold. To maintain the association of the T cell engager with HSA, the patient can be dosed regularly with the CID small molecule, and the frequency of dosing depends on combination of the CID small molecule’s serum half-life, and the lifetime of the CID complex.

In the event that the patient needs to have the T cell engager cleared quickly, for example, due to safety concerns, the patient would stop being dosed with the small molecule, leading to clearance of the small molecule, disassociation of the T cell engager from HSA, and rapid clearance of the T cell engager in the patient. The rate of clearance of the T cell engager depends on a combination of the small molecule’s serum half-life, the binding affinity of the CID small molecule to the first and second CID domains, the lifetime of the CID complex, and the clearance rate of the T cell engager when no longer associated with HSA.

The method described above enables a precise temporal control of the serum half-life of a T cell engager domain in a patient, and the method is applicable to patients suffering from a variety of diseases or conditions, for example, a proliferative disease, and a tumorous disease. Depending on the disease of a patient and the cells envisioned to be targeted by cytotoxic T cells, an appropriate T cell engager domain can be designed to be incorporated in the composition described herein. For example, to treat and control the serum half-life of the T cell engager domain in patients suffering from most of B cell malignancies including but not limited to acute lymphoblastic leukemia (ALL), chronic lymphocytic leukemia (CLL) and B cell lymphomas, a T cell engager domain comprising a CD19 ABD and a CD3 ABD can be incorporated in the composition described herein.

Administration of the composition described herein may be done in a variety of ways, including, but not limited to intravenously or locally.

The dosing amounts and frequencies of administration are, in a preferred embodiment, selected to be therapeutically or prophylactically effective. As is known in the art, dosages for any one patient depends on many factors, the age, body weight, general health, sex, diet, time and route of administration, drug interaction and the severity of the condition may be necessary, and will be ascertainable with routine experimentation by those skilled in the art.

### EXAMPLES

The invention now being generally described, will be more readily understood by reference to the following examples, which are included merely for purposes of illustration of certain aspects and embodiments of the present invention, and is not intended to limit the invention.

### A. Example 1

Fusion protein moieties were constructed to include a second CID domain (AZ21) and a therapeutic domain (a T cell engager domain comprising an anti-CD3 antigen binding domain and an anti-CD19 antigen binding domain). Different formats are shown in FIG. 6. The ability of these fusion protein moieties to activate T cells were tested.

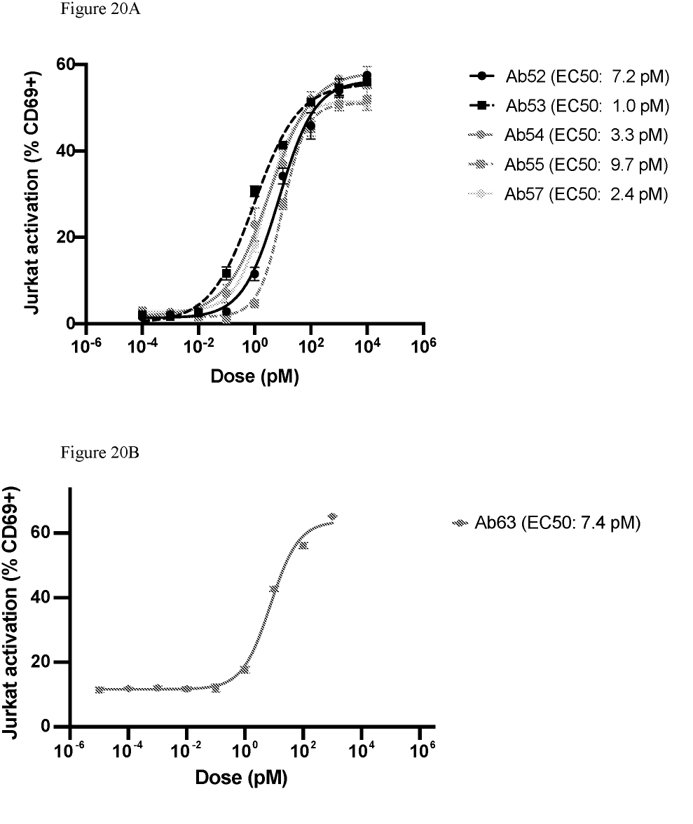

Raji B lymphoma cells were labeled with CFSE-DA (Tonbo, 750 nM final concentration) in DPBS for 10 minutes, then washed with RPMI + 10% FBS media. Raji B cells were then mixed with Jurkat T cells at a 10:1 effector:target ratio and seeded in U-bottom 96-well plates at 1.1e5 cells total per well. Serially diluted fusion protein moieties were added to the cells and incubated 16 hours at 37° C. with 5% CO2. After incubation, cells were stained for viability by adding a membrane-impermeable protein-reactive dye (Biotium CF405S-SE, 1.5 µM final concentration) for 10 minutes at 37° C. Cells were then immediately fixed by adding paraformaldehyde (Electron Microscopy Sciences, 1.6% final concentration) for 10 minutes. Cells were centrifuged, supernatant was aspirated, then pellets were resuspended in 50 µL PE-conjugated anti-human CD69 (BioLegend, clone FN50) and stained 30 minutes at room temperature before measuring by flow cytometry. Viable Jurkat cells (CF405S-negative, CFSE-negative) were assessed for CD69 expression by flow cytometry and manual gating. The frequency of CD69+ cells is expressed as a percent of viable Jurkat cells. Non-linear (logistic) fit curves and EC50 values were calculated in GraphPad Prism 8 software. As shown in FIGS. 20A-20B, Ab52, Ab53, Ab54, Ab55, Ab57, and Ab63 activated T cells.

### B. Example 2

Jurkat/Raji co-culture assays were performed and Jurkat CD69 expression was measured by flow cytometry as described in Example 1, with the following modifications: Ab59 (anti-HSA BCL2 fusion protein) was added to all wells at an equimolar concentration to the fusion protein moieties being titrated. Cells were then incubated for 10 minutes at 37° C. before adding 10 nM of either ABT-199 (venetoclax, filled circles) or vehicle control (DMSO, open circles). EC50 was measured under each condition.

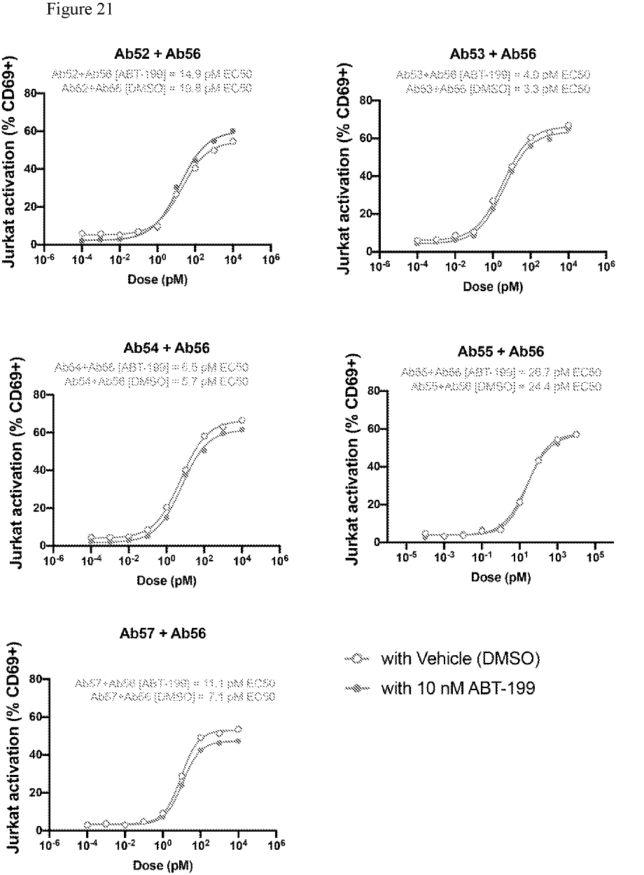

As shown in FIG. 21, the ability of the fusion protein moieties to activate T cells were not affected significantly after complexing with anti-HSA BCL2 fusion protein.

### C. Example 3

The ability of fusion protein moieties to induce T cell cytotoxicity towards Raji B lymphoma cells were examined. Raji B lymphoma cells were labeled with CFSE-DA (Tonbo, 750 nM final concentration) in DPBS for 10 minutes, then washed with RPMI + 10% FBS media. Raji B cells were then mixed with magnetically isolated primary human T cells (magnetically isolated with EasySep Human T cell isolation kit, negative selection; StemCell Technologies Cat 17951) at a 10:1 effector:target ratio and seeded in U-bottom 96-well plates at 1.1e5 cells total per well. Serially diluted fusion protein moieties were added and cells were incubated 42 hours at 37° C. with 5% CO2. After incubation, cells were stained for viability by adding a membrane-impermeable protein-reactive dye (Biotium CF405S-SE, 1.5 µM final concentration) for 10 minutes at 37° C. Cells were then immediately fixed by adding paraformaldehyde (Electron Microscopy Sciences, 1.6% final concentration) for 10 minutes. Cells were centrifuged, supernatant was aspirated, then pellets were resuspended in 75 µL AutoMACS Running Buffer (Miltenyi) before measuring by flow cytometry. Raji cells (CF405S-positive, CFSE-positive) were identified by manual gating. The frequency of dead (CF405S+) Raji cells is expressed as a percent of all Raji cells. Non-linear (logistic) fit curves and EC50 values were calculated in R: A Language and Environment for Statistical Computing using the dr4pl package.

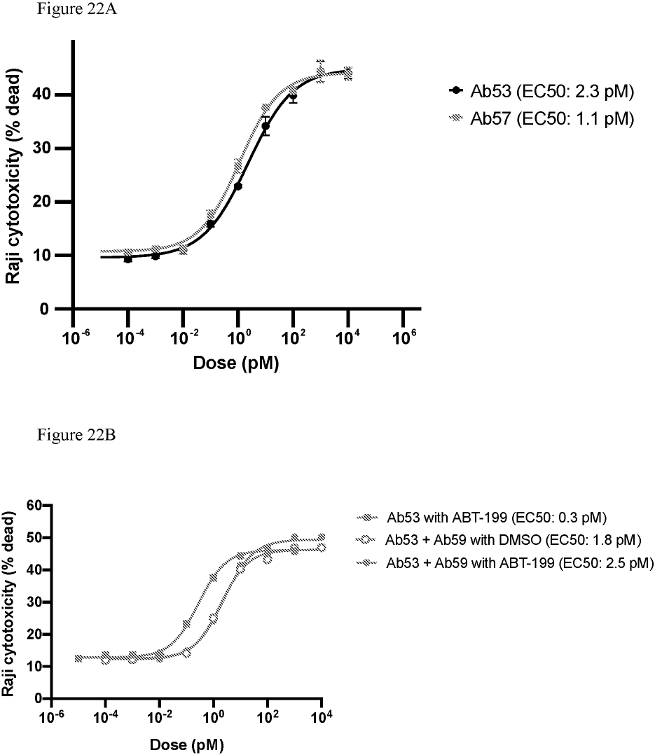

As shown in FIG. 22A, fusion protein moieties Ab53 and Ab57 induced T cell cytotoxicity towards Raji B lymphoma cells.

Primary T cell/Raji co-culture assays were performed and cytotoxicity was measured by flow cytometry as described above, with the following modifications: Ab59 (human IgG1 FC BCL2 fusion protein) was added to some wells (circles shown in FIG. 22B) at an equimolar concentration to the antibody being titrated. Cells were incubated 10 minutes at 37° C. after adding Ab59, then either ABT-199 (venetoclax, filled symbols in FIG. 22B) or vehicle control (DMSO, open symbols in FIG. 22B) was added at a final concentration of 10 nM. The co-culture incubation time was 40 hrs for this experiment. As shown in FIG. 22B, the ability of the fusion protein moieties to induce T cell cytotoxicity were not affected significantly after complexing with anti-HSA BCL2 fusion protein.

### D. Example 4

The ability of fusion protein moieties containing human IL-2 to activate the STAT5 transcription factors in T cells were assayed and compared with human IL-2.

Primary human T cells (magnetically isolated with EasySep Human T cell isolation kit, negative selection; StemCell Technologies Cat 17951) were resuspended in RPMI + 10% FCS media at 1e6 cells/mL and treated with the indicated concentrations of human interleukin-2 (IL-2) or fusion protein moieties containing human IL-2 for 15 minutes at 37° C. Ab93 is human IL-2 fused to the C-terminus of a single-chain Fv antibody fragment of the AZ21. Ab94 is human IL-2 fused to the N-terminus of a single-chain Fv antibody fragment of the AZ21. After incubation, cells were immediately fixed by adding paraformaldehyde (Electron Microscopy Sciences, 1.6% final concentration) for 10 minutes. Cells were centrifuged, supernatant was aspirated, then pellets were resuspended in ice-cold 100% methanol for 10 minutes at 4° C. The methanol was diluted with an equal volume of AutoMACS Running Buffer (Miltenyi) and cells were centrifuged again. Cells were washed again with AutoMACS Running Buffer and then stained with AlexaFluor647-conjugated anti-human phospho-STAT5 (pY694) (BD Biosciences, clone 47) for 30 minutes at room temperature. Cells were washed again twice before measuring by flow cytometry. The abundance of phosphorylated STAT5 is expressed as median fluorescence intensity among singlet-gated cells. Median values were calculated in Cytobank software (cytobank.org).

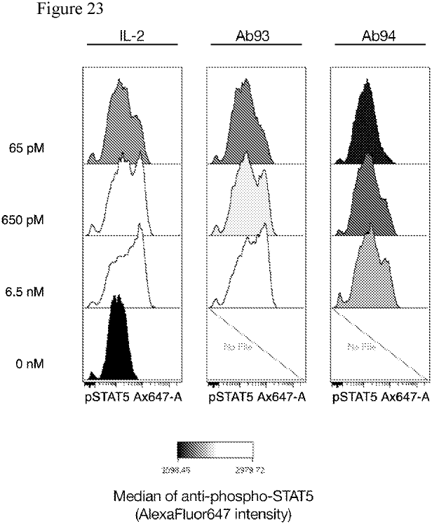

As shown in FIG. 23, fusion protein moieties containing human IL-2 are capable of activating the STAT5 transcription factors in T cells to the similar extent as human IL-2.

### E. Example 5

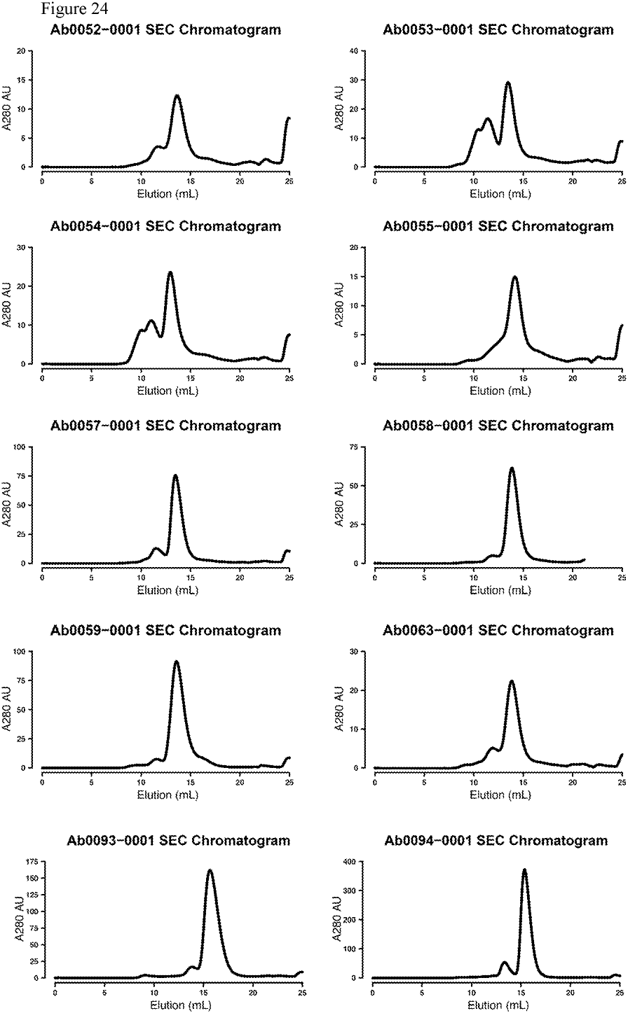

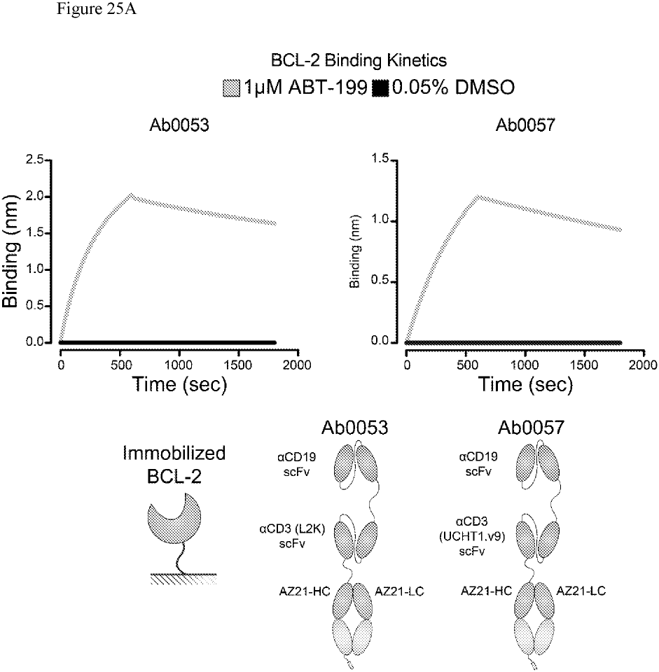

Fusion protein moieties which are his-tagged were purified via Ni-NTA resin. After purification, the fusion protein moieties were further separated by size exclusion chromatography run on a Superdex® 200 Increase 10/300 GL column monitored under UV 280 nm. Size exclusion chromatography chromatogram for each fusion protein moiety was shown in FIG. 24.

### F. Example 6

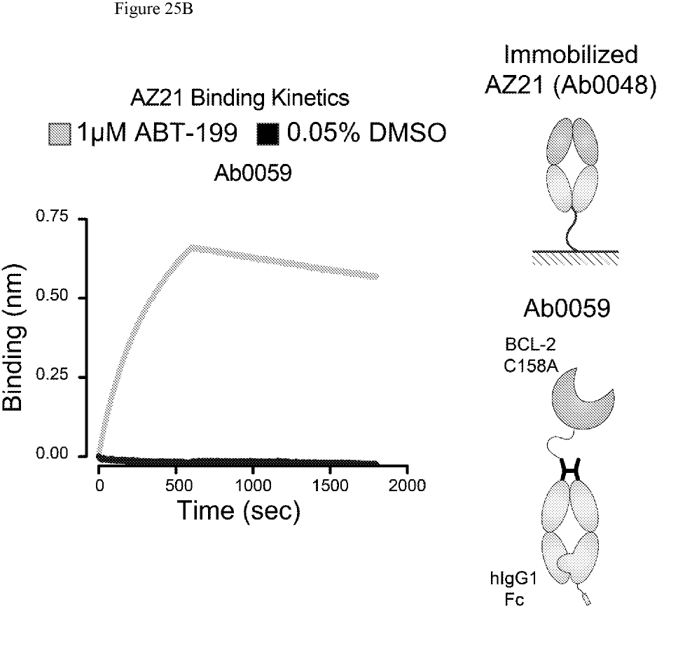

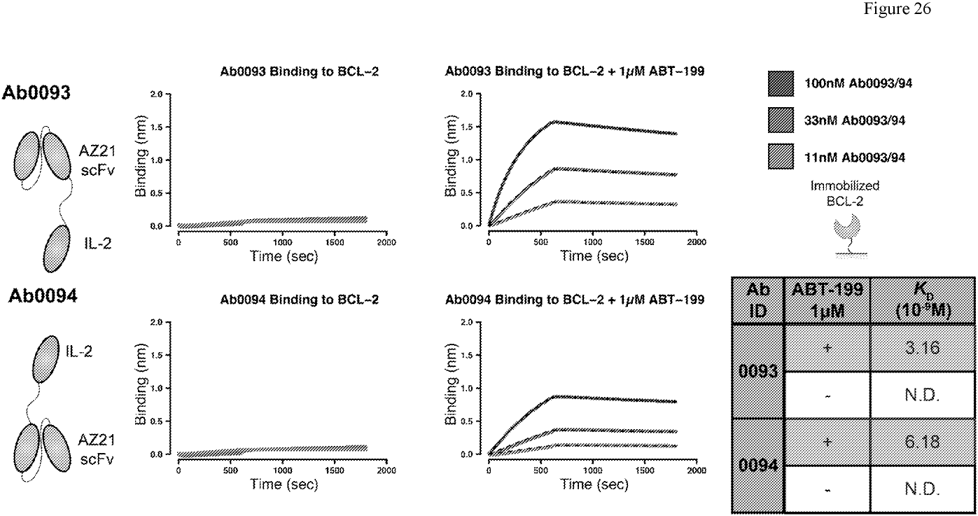

Binding kinetics between fusion protein moieties and BCL-2 was assayed by biolayer interferometry. As shown in FIG. 25A, Ab53 and Ab57 showed potent and reversible binding to BCL-2 in the presence of ABT-199 (grey line) and no significant binding was observed in the absence of ABT-199 (black line). As shown in FIG. 26, Ab93 and Ab94 showed potent and reversible binding to BCL-2 in the presence of ABT-199 (Right) and no significant binding was observed in the absence of ABT-199 (Left). Curves represent measured data points on an Octet RED384. KDs were also calculated.

Binding kinetics between BCl-2 fusion protein Ab59 and AZ-21 was assayed by biolayer interferometry. As shown in FIG. 25B, Ab59 showed potent and reversible binding to AZ21 in the presence of ABT-199 (grey) and no significant binding was observed in the absence of ABT-199 (black). Curves represent measured data points on an Octet RED384.

### G. Example 7: Bcl-2 CID Domain Dimerizes with AZ21 CID Domain

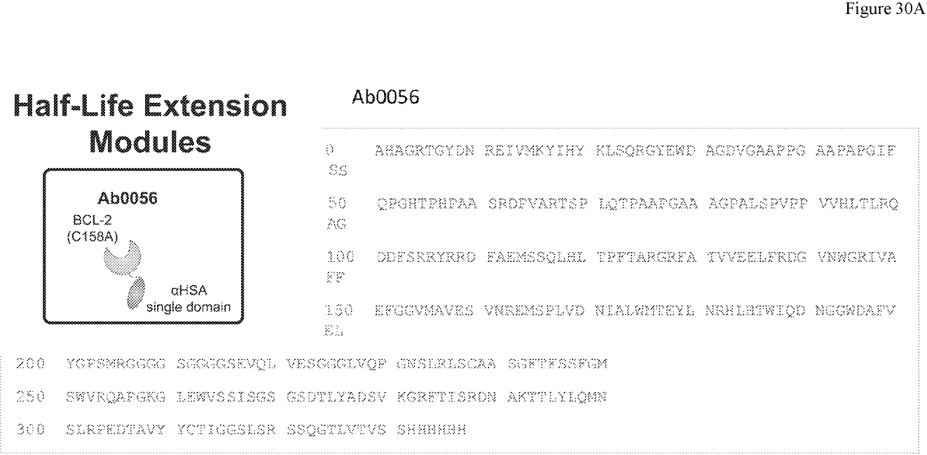

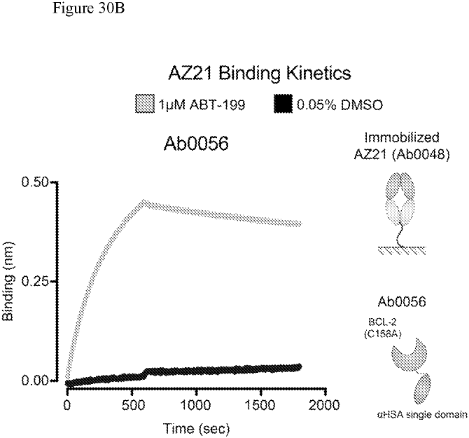

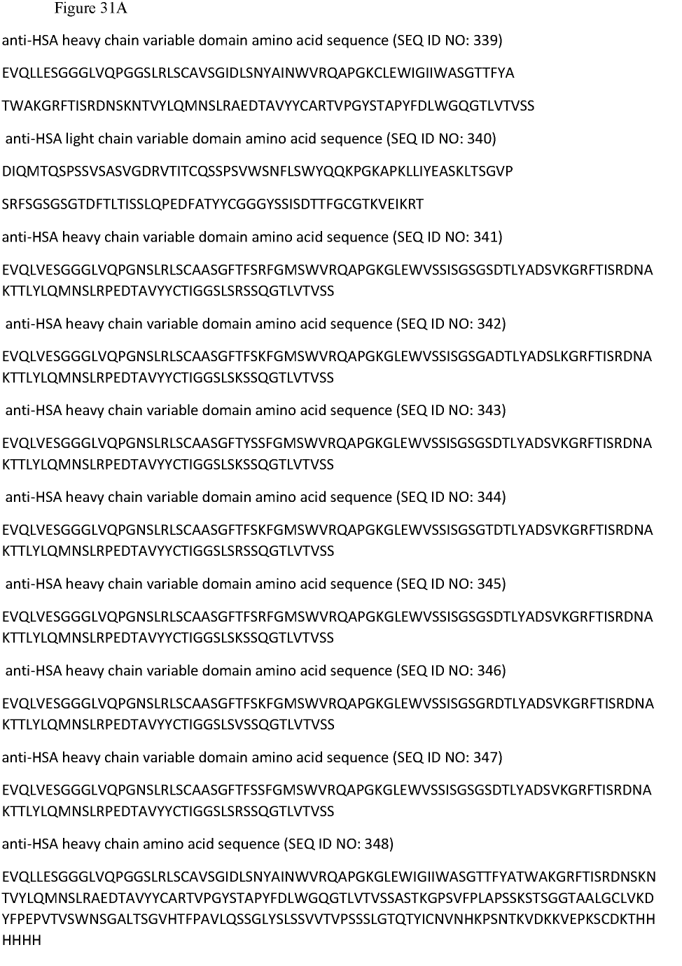

Binding of a CID domain Bcl-2 (C158A) to its cognate CID domain AZ21 was tested in the presence or absence of the CID small molecule ABT-199. A monomer composed of BCL-2 (C158A) linked with a single domain anti-HSA antibody (sequence see FIG. 30A) was produced and purified in CHO cells. The cognate CID domain AZ21 was produced in the Fab format, and comprises vh-CDR1 (SEQ ID NO:1), vh-CDR2 (SEQ ID NO:72), vh-CDR3 (SEQ ID NO:129), vl-CDR1 (SEQ ID NO:310), vl-CDR2 (SEQ ID NO:311), and vl-CDR3 (SEQ ID NO:223). AZ21 was immobilized, and binding dynamics of the monomer to AZ21 was measured by Octet RED 384 in the presence or absence of 1µM ABT-199. FIG. 30B shows that ABT-199 mediates the binding of Bcl-2 (C158A) to its cognate CID domain AZ21, and no significant binding was observed in the absence of ABT-199.

### H. Example 8: Ab57+Ab59 PK Study

Ten C57BL/6J 6-week-old male mice were randomized into 2 groups of 5 mice each. Six days prior to antibody administration, 25 µL blood samples were collected from all mice as pretreatment control samples, processed to plasma, diluted 1/10 in 50% glycerol in PBS, frozen in specialized 96 well storage plates, and stored at -20° C. Two hours prior to antibody administration, Group 1 mice were dosed (oral gavage) with venetoclax at 5 mg/kg, 5 ml/kg, and Group 2 mice were dosed (oral gavage) with vehicle at 5 ml/kg. Mice were repeat dosed with these compounds at 22, 46, 70, 94, 142 and 166 hours after antibody administration. At 0 hours on Day 0, Ab93 and Ab59 were combined and IV injected at 0.15 mg/kg and 5 mg/kg respectively and 5 ml/kg total. 25 µL blood samples were collected from all mice at 3 m, 30 m, 1 h, 2 h, 6 h, 1 d, 3 d, and 7 days. The blood samples were processed as described above. Plasma samples were assessed by ELISA. A human IgG ELISA (Mabtech) was used to measure Ab59 concentrations. A custom human Fab ELISA (mouse anti-human IgG Kappa capture antibody, BioLegend and goat anti-human Fab detection antibody, Jackson ImmunoResearch) was used to measure Ab57 concentrations. PK analysis was on the plasma concentrations generated from the ELISAs for Ab93 and Ab59 using PK Solutions software.

### I. Example 9: Ab93+Ab59 PK Study

Ten C57BL/6J 6-week-old male mice were randomized into 2 groups of 5 mice each. Six days prior to antibody administration, 25 µL blood samples were collected from all mice as pretreatment control samples, processed to plasma, diluted 1/10 in 50% glycerol in PBS, frozen in specialized 96 well storage plates, and stored at -20° C. Two hours prior to antibody administration, Group 1 mice were dosed (oral gavage) with venetoclax at 5 mg/kg, 5 ml/kg, and Group 2 mice were dosed (oral gavage) with vehicle at 5 ml/kg. Mice were repeat dosed with these compounds at 22, 46, 70, 94, 142 and 166 hours after antibody administration. At 0 hours on Day 0, Ab93 and Ab59 were combined and IV injected at 0.15 mg/kg and 5 mg/kg respectively and 5 ml/kg total. 25 µL blood samples were collected from all mice at 3 m, 30 m, 1 h, 2 h, 6 h, 1 d, 3 d, and 7 days. The blood samples were processed as described above. Plasma samples were assessed by ELISA. A human IgG ELISA (Mabtech) was used to measure Ab59 concentrations and human IL-2 ELISA (R&D Systems) was used to measure Ab93 concentrations. PK analysis was on the plasma concentrations generated from the ELISAs for Ab93 and Ab59 using PK Solutions software.

1. A composition comprising:
(1) a heterodimeric Fc fusion protein comprising:
a) a first monomer comprising a first CID domain and a first Fc domain of an IgG, wherein said first CID domain is covalently linked to said first Fc domain, and
b) a second monomer comprising a second Fc domain of said IgG;
(2) a fusion protein moiety comprising a second CID domain and a therapeutic moiety, wherein said second CID domain is covalently linked to said therapeutic moiety at N or C terminus,
wherein in the presence of a CID small molecule said first CID domain and said CID second domain form a complex of said first CID domain-said CID small molecule-said second CID domain.

2. The composition according to claim 1, wherein said CID small molecule is selected from the group consisting of FK1012, rimiducid, FK506, FKCsA, Rapamycin, Rapamycin analogs, Courmermycin, Gibberellin, HaXS, TMP-tag, ABT-737.

3. The composition according to claim 2, wherein said complex of said first CID domain-said CID small molecule-said second CID domain is selected from the group of complexes consisting of FKBP-FK1012-FKBP, variant FKBP-rimiducid-variant FKBP, FKBP-FK506-Calcineurin, FKBP-FKCsA-CyP-Fas, FKBP-Rapamycin-FRB, variant FKBP-Rapamycin analogs-variant FRB, GyrB-Courmermycin-GyrB, GAI-Gibberellin-GID1, SNAP-tag-HaXS-HaloTag, eDHFR-TMP-tag-HaloTag and AZ1-ABT-737-BCL-xL, wherein said first CID domain and said second CID domain can swap positions within said complex.

4. The composition according to claim 1, wherein said first CID domain comprises a heavy chain variable domain and a light chain variable domain, and said second CID domain comprises a heavy chain variable domain and a light chain variable domain capable of binding to the complex formed between said first CID domain and said CID small molecule.

5. The composition according to claim 4, wherein said small molecule is methotrexate.

6. The composition according to claim 1, wherein said first CID domain is BCL-2 or variants thereof, said CID small molecule is ABT-199 or ABT-263, and said second CID domain comprises a heavy chain variable domain and a light chain variable domain capable of binding to the complex formed between said first CID domain and said CID small molecule.

7. The composition according to claim 6, wherein said first CID domain is BCL-2 or BCL-2 (C158A), said CID small molecule is ABT-199, and said second CID domain comprises:
a) a variable heavy domain (VH) comprising:
i) a vhCDR1 comprising SEQ ID NO:1;
ii) a vhCDR2 comprising SEQ ID NO: 72; and
iii) a vhCDR3 comprising SEQ ID NO:129; and
b) a variable light domain (VL) comprising:
i) a vlCDR1 comprising SEQ ID NO:310;
ii) a vlCDR2 comprising SEQ ID NO:311; and
iii) a vlCDR3 comprising SEQ ID NO:233.

8. The composition according to claim 1, wherein said first CID domain is an ABT-737 binding domain of Bcl-xL, said CID small molecule is ABT-737, and said second CID domain comprises a heavy chain variable domain and a light chain variable domain capable of binding to the complex formed between said first CID domain and said CID small molecule.

9. The composition according to claim 1, wherein said first CID domain is a rapamycin binding domain of FKBP, said CID small molecule is rapamycin, and said second CID domain comprises a heavy chain variable domain and a light chain variable domain capable of binding to the complex formed between said first CID domain and said CID small molecule.

10. The composition according to claim 1, wherein said first CID domain is an GDC-0152, LCL161, AT406, CUDC-427, or Birinapant binding domain of cIAPl, said CID small molecule is GDC-0152, LCL161, AT406, CUDC-427, or Birinapant, and said second CID domain comprises a heavy chain variable domain and a light chain variable domain capable of binding to the complex formed between said first CID domain and said CID small molecule.

11. The composition according to claim 1, wherein said first CID domain is thalidomide binding domain of cereblon, said small molecule is thalidomide, lenalidomide, or pomalidomide, and said second CID domain comprises a heavy chain variable domain and a light chain variable domain capable of binding to the complex formed between said first CID domain and said CID small molecule.

12. The composition according to any one of the claims 1-11, wherein said therapeutic moiety is selected from an antibody, an antibody fragment, a cytokine, a hormone, a peptide, and an antibody drug conjugate.

13. The composition according to claim 12, wherein said therapeutic moiety is a bispecific antibody.

14. The composition according to claim 13, wherein said therapeutic moiety is a bispecific T cell engager moiety.

15. The composition according to claim 14, wherein said bispecific T cell engager moiety comprises a T cell antigen-binding domain and a tumor-associated antigen-binding domain.

16. The composition according to claim 15, wherein said T cell antigen is CD3 and said tumor-associated antigen is CD19.

17. The composition according to claim 12, wherein said therapeutic moiety is a human interleukin molecule.

18. The composition according to claim 17, wherein said therapeutic moiety is human IL-2.

19. The composition according to any one of the claims 1-18, wherein said first CID domain is linked to said first Fc domain via a first linker.

20. The composition according to any one of the claims 1-19, wherein said second CID domain is linked to said therapeutic moiety via a second linker.

21. The composition according to any one of the claims 1-20, wherein said IgG is human IgG1.

22. The composition according to any one of the claims 1-21, wherein said first Fc domain is a first variant Fc domain, and said second Fc domain is a second variant Fc domain.

23. A method of extending serum half-life of a therapeutic moiety in a patient, the method comprising:
a) administering to said patient said composition comprising said therapeutic moiety according to any one of the claims 1-22;
b) administering to said patient said CID small molecule according to any of the claims 1-22;
wherein said first and second CID domains form complex with said small molecule in said patient, and whereby serum half-life of said therapeutic moiety is extended.

24. A method of clearing a therapeutic moiety from a patient, wherein said patient has been administered said composition comprising said therapeutic moiety and said CID small molecule according to any of the claims 1-22, the method comprising ceasing administration of said CID small molecule to said patient, such that said therapeutic moiety is cleared from said patient’s blood.

25. A composition comprising:
(1) a heterodimeric Fc fusion protein comprising:
a) a first monomer comprising a first CInD domain and a first Fc domain of IgG, wherein said first CInD domain is covalently linked to
said first Fc domain, and
b) a second monomer comprising a second Fc domain of IgG;
(2) a fusion protein moiety comprising a second CInD domain and a second therapeutic moiety, wherein said second CInD domain is covalently linked to said second therapeutic moiety at N or C terminus,
wherein said first CInD domain binds to said second CInD domain forming a complex, and wherein said complex can be disrupted by a CInD small molecule.

26. The composition according to claim 25, wherein either said first CInD domain or said second CInD domain comprises an antibody moiety.

27. The composition according to claim 25 or 26, wherein said first CInD domain is linked to said first Fc domain via a first linker.

28. The composition according to any of the claims 25-27, wherein said second CInD domain is linked to said therapeutic moiety via a second linker.

29. The composition according to any one of the claims 25-28, wherein said IgG is human IgG1.

30. The composition according to any one of the claims 25-29, wherein said first Fc domain is a first variant Fc domain, and said second Fc domain is a second variant Fc domain.

31. The composition according to any one of the claims 25-30, wherein said second therapeutic moiety is selected from an antibody, an antibody fragment, a cytokine, a hormone, a polypeptide, and an antibody drug conjugate.

32. The composition according to claim 31, wherein said second therapeutic moiety is a bispecific antibody.

33. The composition according to claim 32, wherein said second therapeutic moiety is a bispecific T cell engager moiety.

34. The composition according to claim 33, wherein said bispecific T cell engager moiety comprises a T cell antigen-binding domain and a tumor-associated antigen-binding domain.

35. The composition according to claim 34, wherein said T cell antigen is CD3 and said tumor-associated antigen is CD19.

36. The composition according to claim 31, wherein said second therapeutic moiety is a human interleukin molecule.

37. The composition according to claim 36, wherein said second therapeutic moiety is human IL-2.

38. The composition according to any of the claims 25-31, wherein said second monomer further comprises a first therapeutic moiety covalently linked to said second Fc domain.

39. The composition according to claim 38, wherein said first therapeutic moiety is selected from an antibody, an antibody fragment, a cytokine, a hormone, a polypeptide, and an antibody drug conjugate.

40. The composition according to claim 39, wherein said first therapeutic moiety is a T cell antigen-binding domain and said second therapeutic moiety is a tumor-associated antigen-binding domain, or wherein said first therapeutic moiety is a tumor-associated antigen-binding domain and said second therapeutic moiety is a T cell antigen-binding domain.

41. The composition according to claim 40, wherein said T cell antigen is CD3 and said tumor-associated antigen is CD19.

42. A composition comprising:
(1) a homodimeric Fc fusion protein comprising two identical monomers, wherein said two monomers each comprising a first CInD domain covalently linked to a Fc domain of IgG;
(2) a fusion protein moiety comprising a second CInD domain and a therapeutic moiety, wherein said second CInD domain is covalently linked to said therapeutic moiety at N or C terminus,
wherein said first CInD domain binds to said second CInD domain forming a complex, and wherein said complex can be disrupted by a CInD small molecule.

43. The composition according to claim 42, wherein either said first CInD domain or said second CInD domain comprises an antibody moiety.

44. The composition according to claim 42 or 43, wherein said first CInD domain is linked to said first Fc domain via a first linker.

45. The composition according to any of the claims 42-44, wherein said second CInD domain is linked to said therapeutic moiety via a second linker.

46. The composition according to any one of the claims 42-45, wherein said IgG is human IgG1.

47. The composition according to any one of the claims 42-46, wherein said therapeutic moiety is selected from an antibody, an antibody fragment, a cytokine, a hormone, a polypeptide, and an antibody drug conjugate.

48. The composition according to claim 47, wherein said therapeutic moiety is a bispecific antibody.

49. The composition according to claim 48, wherein said therapeutic moiety is a bispecific T cell engager moiety.

50. The composition according to claim 49, wherein said bispecific T cell engager moiety comprises a T cell antigen-binding domain and a tumor-associated antigen-binding domain.

51. The composition according to claim 50, wherein said T cell antigen is CD3 and said tumor-associated antigen is CD19.

52. The composition according to claim 47, wherein said therapeutic moiety is a human interleukin molecule.

53. The composition according to claim 52, wherein said therapeutic moiety is human IL-2.

54. A method of extending serum half-life of a therapeutic moiety in a patient, the method comprising administering to said patient the composition according to any one of the claims 25-53.

55. A method of clearing a therapeutic moiety from a patient, wherein said patient has been previously administered the composition according to any one of the claims 25-53, the method comprising administering said CInD small molecule according to claims 25-53, whereby said therapeutic moiety disassociates from said heterodimeric or homodimeric Fc fusion protein.

56. A composition comprising:
(a) a first monomer comprising:
i) a first CID domain;
ii) an optional domain linker; and
iii) a human serum albumin (HSA) binding domain; and
(b) a second monomer comprising:
i) a second CID domain;
ii) an optional domain linker; and
iii) a T cell engager comprising:
A) a CD3 antigen binding domain (ABD);
B) an optional domain linker; and
C) a tumor-associated antigen (TAA) ABD (TAA-ABD);
Wherein in the presence of a CID small molecule said first CID domain and said second CID domain form a complex of said first CID domain-said CID small molecule-said second CID domain.

57. The composition according to claim 56, wherein said CID small molecule is selected from the group consisting of FK1012, rimiducid, FK506, FKCsA, Rapamycin, Rapamycin analogs, Courmermycin, Gibberellin, HaXS, TMP-tag, ABT-737.

58. The composition according to claim 57, wherein said complex of said first CID domain-said CID small molecule-said second CID domain is selected from the group of complexes consisting of FKBP-FK1012-FKBP, variant FKBP-rimiducid-variant FKBP, FKBP-FK506-Calcineurin, FKBP-FKCsA-CyP-Fas, FKBP-Rapamycin-FRB, variant FKBP-Rapamycin analogs-variant FRB, GyrB-Courmermycin-GyrB, GAI-Gibberellin-GID1, SNAP-tag-HaXS-HaloTag, eDHFR-TMP-tag-HaloTag, AZ1-ABT-737-BCL-xL, Calcineurin-FK506-FKBP, CyP-Fas-FKCsA-FKBP, FRB-Rapamycin-FKBP, variant FRB-Rapamycin analogs-variant FKBP, GID1-Gibberellin-GAI, HaloTag-HaXS-SNAP-tag, HaloTag-TMP-tag-eDHFR, BCL-xL-ABT-737-AZ1.

59. The composition according to any one of the claims 56-58, wherein said HSA binding domain comprises a heavy chain variable domain and a light chain variable domain, or a single monomeric variable antibody domain.

60. The composition according to any of the claims 56-59, wherein said first CID domain is linked to said HSA binding domain via a first linker, and said second CID domain is linked to said T cell engager via a second linker.

61. A pharmaceutical composition comprising a composition according to any one of the claims 56-60.

62. A method of extending serum half-life of a T cell engager in a patient, the method comprising:
a) administering to said patient said composition or said pharmaceutical composition comprising said T cell engager according to any one of the claims 56-61;
b) administering to said patient said small molecule drug according to any of the claims 56-61;
wherein said first and second CID domains form complex with said small molecule in said patient, and whereby serum half-life of said T cell engager is extended.

63. A method of treating cancer in a patient, the method comprising:
administering to said patient said composition or said pharmaceutical a) administering to said pharmaceutical composition comprising said T cell engager according to any one of the claims 56-61;
b) administering to said patient said small molecule according to any of the claims 56-61;
wherein said first and second CID domains form complex with said small molecule in said patient to treat cancer.

64. A method of clearing a T cell engager from a patient, wherein said patient has been administered said composition comprising said T cell engager and said small molecule according to any of the claims 56-61, the method comprising stopping administration of said small molecule to said patient, such that said T cell engager is cleared from said patient’s blood.

65. A method of treating cancer in a patient, the method comprising:
a) administering to said patient said composition or said pharmaceutical composition comprising said T cell engager according to any one of the claims 1-53;
b) administering to said patient said small molecule according to any of the claims 1-53;
wherein said first and second CID domains form complex with said small molecule in said patient to treat cancer.
